(19) United States
(12) Patent Application Publication
(10) Pub. No.: US 2020/0056145 A1
(43) Pub. Date: Feb. 20, 2020

(54) ENGINEERED COMMENSAL BACTERIA AND METHODS OF USE

(71) Applicant: The Regents of the University of California, Oakland, CA (US)

(72) Inventors: Steven Brown, La Jolla, CA (US); Amir Zarrinpar, La Jolla, CA (US); Juliet Snyder, La Jolla, CA (US)

(21) Appl. No.: 16/604,138

(22) PCT Filed: Apr. 17, 2018
(86) PCT No.: PCT/US18/27998

§ 371 Date: Oct. 9, 2019

Related U.S. Application Data
(60) Provisional application No. 62/486,068, filed on Apr. 17, 2017.

(51) Int. Cl. C12N 1/20; A61K 35/742; A61K 38/50; A61K 9/48; C12N 9/80

(52) U.S. Cl. CPC C12N 1/20; A61K 35/742; C12Y 305/01024; A61K 9/4866; C12N 9/80; A61K 38/50

(57) ABSTRACT

Provided are compositions comprising populations of commensal bacteria isolated from a microbiome sample of a mammalian subject and engineered to express a heterologous polynucleotide, compositions comprising such engineered commensal bacteria and methods of use for delivering a therapeutic polypeptide to a mammal, e.g, by administering the engineered commensal/native bacteria.

### CROSS-REFERENCE TO RELATED APPLICATIONS

This application claims the benefit under 35 U.S.C. § 119(e) of U.S. Provisional Application No. 62/486,068, filed on Apr. 17, 2017, which is hereby expressly incorporated by reference herein in its entirety for all purposes.

### STATEMENT OF GOVERNMENTAL SUPPORT

This work was supported in part by Grant Nos. R03DK114536 and 1K08DK10290201A1, from The National Institutes of Health. The Government has certain rights in this invention.

### BACKGROUND

Recent advances in sequencing, mass spectrometric, and bioinformatic technologies have facilitated our understanding of the role the microbiome plays in host physiological processes including, but not limited to, metabolism, inflammation, behavior and neurological disease (Vuong, et al., Annu Rev Neurosci. (2017) 40:21-49) [also cite 25271724]. Though it is conceptually exciting to link physiological processes to the microbiome, the field is still nascent; studies show strong associations but few show a clear mechanistic relationship between the microbiota and various physiological processes. For example, the sheer quantity of analytes that are modulated by the gut microbiome, as well as signaling molecules from the gut epithelium, makes it difficult to identify microbiome functions that may be contributing to neurobehavioral processes. To develop a better mechanistic understanding and more effective microbiome-mediated therapies, a different approach stressing a functional modulation of the gut microbiome is necessary.

### SUMMARY

In one aspect, provided are methods of delivering a therapeutic polypeptide to a mammalian subject in need thereof. In some embodiments, the methods comprise:

a) obtaining a microbiome sample comprising bacterial cells from a donating subject;

b) isolating a bacterial cell from the microbiome sample, wherein the isolated bacteria cell is from a bacterial strain that is commensal/native to the donating subject;

c) culturing the isolated bacteria cell in vitro to yield a substantially homogeneous population of the isolated and cultured bacteria cells;

d) transforming the substantially homogeneous population with one or more polynucleotides that are heterologous to the bacteria and/or the donating subject, wherein the one or more polynucleotides encode one or more therapeutic polypeptides; and

e) administering or causing to be administered to a receiving subject at least a portion of the substantially homogeneous and transformed population of the isolated and cultured bacteria cells, e.g., in a therapeutically sufficient amount, wherein the administered bacteria cells are capable of colonizing, or are configured to colonize in or on the mammalian subject, permanently or long-term in or on the mammalian subject and express the one or more therapeutic polypeptide, e.g., at levels sufficient to exert a therapeutic effect on the mammal. In some embodiments, the methods further comprise the step of determining and/or measuring the colonization or presence of the administered bacteria cells in or on said mammalian subject. In some embodiments, the microbiome sample is obtained from the a biological sample selected from the group consisting of a bodily excretion (e.g., feces, saliva, mucus, urine, breath), a biopsy or swab of a surface (e.g., gastrointestinal (GI) tract, oral cavity, pharynx, nasal cavity, urogenital track, skin, anus/rectum, vagina, eye) and a pathological specimen (e.g., cancerous tissue, amputated limbs, inflamed organs). In some embodiments, the bacterial cell does not comprise a polynucleotide encoding for a pathogenic toxin. In some embodiments, the bacterial cell does not or population of bacterial cells do not comprise one or more polynucleotides encoding for one or more pathogenic toxins selected from the group consisting of AB toxin, Alpha toxin, Anthrax toxin, Botulinum toxin, Cereulide, Cholesterol-dependent cytolysin, Clostridial Cytotoxin family, Clostridium botulinum C3 toxin, Clostridium difficile toxin A, Clostridium difficile toxin B, Clostridium enterotoxin, Clostridium perfringens alpha toxin, Clostridium perfringens beta toxin, Cry1Ac, Cry6Aa, Cry34Ab1, Delta endotoxin, Diphtheria toxin, Enterotoxins, Enterotoxin type B, Erythrogenic toxin, Exfoliatin, Fragilysin, Haemolysin E, Heat-labile enterotoxin, Heat-stable enterotoxin, Hemolysin, HrpZ Family, Leukocidin, Listeriolysin O, Panton-Valentine leucocidin, intact Pathogenicity island, Phenol-soluble modulin, Pneumolysin, Pore-forming toxin, Pseudomonas exotoxin, Pyocyanin, anti-eukaryotic Rhs toxins, RTX toxin, Shiga toxins, Shiga-like toxin, Staphylococcus aureus alpha toxin, Staphylococcus aureus beta toxin, Staphylococcus aureus delta toxin, Streptolysin, Tetanolysin, Tetanospasmin, Toxic shock syndrome toxin, Tracheal cytotoxin, and/or Verocytotoxin. In some embodiments, the bacterial cell is or population of bacterial cells are antibiotic sensitive to one or more antibiotic agents used for selection of transformed bacterial cells, e.g., kanamycin, chloramphenicol, carbenicillin, hygromycin and/or trimethoprim. In some embodiments, the bacterial cell is not antibiotic resistant to clinically used antibiotic agents. In some embodiments, the bacterial cell is or population of bacterial cells are not antibiotic resistant to one or more antibiotic agents selected from antibiotic macrolides (e.g., azithromycin, clarithromycin, erythromycin, fidaxomicin, telithromycin, carbomycin A, josamycin, kitasamycin, midecamycin/midecamycin acetate, oleandomycin, solithromycin, spiramycin, troleandomycin, tylosin/tylocine, roxithromycin), rifamycins (e.g., rifampicin (or rifampin), rifabutin, rifapentine, rifalazil, rifaximin), polymyxins (e.g., polymyxin B, polymyxin E (colistin)), quinolone antibiotics (e.g., nalidixic acid, ofloxacin, levofloxacin, ciprofloxacin, norfloxacin, enoxacin, lomefloxacin, grepafloxacin, trovafloxacin, sparfloxacin, temafloxacin, moxifloxacin, gatifloxacin, gemifloxacin), beta-lactams (e.g., penicillin, cloxacillin, dicloxacillin, flucloxacillin, methicillin, nafcillin, oxacillin, temocillin, amoxicillin, ampicillin, mecillinam, carbenicillin, ticarcillin, azlocillin, mezlocillin, piperacillin), aminoglycosides (e.g., amikacin, gentamicin, neomycin, streptomycin, tobramycin), cephalosporins (e.g., cefadroxil, cefazolin, cephalexin, cefaclor, cefoxitin, cefprozil, cefuroxime, loracarbef, cefixime, cefdinir, cefditoren, cefoperazone, cefotaxime, cefpodoxime, cefepime, ceftobiprole), monobactams (e.g., aztreonam, tigemonam, nocardicin A, tabtoxinine β-lactam), carbapenems (e.g., biapenem, doripenem, ertapenem, faropenem, imipenem, meropenem, panipenem, razupenem, tebipenem, thienamycin), and tetracyclines (e.g., tetracycline, chlortetracycline, oxytetracycline, demeclocycline, lymecycline, meclocycline, methacycline, minocycline, rolitetracycline, tigecycline). In some embodiments, the one or more heterologous polynucleotides encode a fluorescent protein, e.g., green fluorescent protein, yellow fluorescent protein, red fluorescent protein (mCherry, mEos2, mRuby2, mRuby3, mClover3, mApple, mKate2, mMaple, mCardinal, or mNeptune), mTurquoise, or mVenus. In some embodiments, the one or more heterologous polynucleotides encode an enzyme, a cytokine or a peptide hormone. In some embodiments, the enzyme is a bile salt hydrolase, e.g., from Lactobacillus, e.g., bshA (Gene ID 3251811) or bshB (Gene ID 3252955), N acylphosphatidylethanolamine (NAPE)-hydrolyzing phospholipase D, Actinobacillus actinomycetemcomitans dispersin B (DspB), lactase (beta-galactosidase), an aldehyde dehydrogenase, an alcohol dehydrogenase (e.g., ADH1A, ADH1B, ADH1C, ADH2, ADH3, ADH4, ADH5, ADH6, ADH7), bile acid-CoA:amino acid N-acyltransferase (BAAT), phenylalanine hydroxylase, butyrate synthesis pathway enzymes, Aspergillus niger-derived prolyl endoprotease (AN-PEP), 7 alpha-hydroxysteroid dehydrogenase (7-alpha-HSDH), 7 beta-hydroxysteroid dehydrogenase (7-beta-HSDH), cholylglycine hydrolase and cholic acid 7alpha-dehydroxylase. In some embodiments, the cytokine is selected from the group consisting of mammalian (e.g., human) IL-10 and mammalian (e.g., human) IL-27 dimer (IL27 alpha subunit and Epstein-Barr virus induced 3 (EBI3) subunit expressed separately or as a fusion protein), and TGF-β. In some embodiments, the peptide hormone is selected from the group consisting of mammalian glucagon, glucagon-like peptide 1 (GLP-1), mammalian glucagon-like peptide 2 (GLP-2), Fibroblast growth factor 1 (FGF1), Fibroblast growth factor 15 (FGF15), Fibroblast growth factor 19 (FGF19), insulin, and proinsulin. In some embodiments, the one or more heterologous polynucleotides encode Akkermansia muciniphila Amuc_1100, Vibrio vulnificus flagellin B, elafin, trefoil factor 1 (TFF1), trefoil factor 2 (TFF2), trefoil factor 3 (TFF3), anti-TNFα antibodies/nanobodies or fragments or single chains thereof, Nostoc elipsosporum cyanovirin-N or microcin J25 (MccJ25). In some embodiments, the one or more heterologous polynucleotides comprise codon bias and/or codon optimization configured to improve or enhance expression of the heterologous protein in the transformed population of the isolated and cultured bacterial cells. In some embodiments, the one or more heterologous polynucleotides are integrated into the chromosome of the bacterial cells of the transformed population. In some embodiments, the one or more heterologous polynucleotides are integrated into the attB and/or yfgG genes of the bacterial genome. In some embodiments, the one or more heterologous polynucleotides are in a plasmid episomally introduced into the bacteria cells of the transformed population. In some embodiments, the transformed bacterial cells further comprise a plasmid retention or maintenance system, e.g., a partitioning system or a toxin-antitoxin module or system. In some embodiments, the one or more heterologous polynucleotides are integrated into an expression cassette having at least or at least about 80%, 85%, 90%, 95%, 97%, 99% or 100% sequence identity to SEQ ID NO:2, and are expressed under the control of a Ptrc promoter. In some embodiments, the heterologous polynucleotide is expressed under the control of a constitutive promoter. In some embodiments, the heterologous polynucleotide is expressed under the control of an inducible promoter. In some embodiments, the bacteria cell is from a gram negative bacterial strain. In some embodiments, the bacteria cell is derived from a bacteria genus selected from the group consisting of Bacteroides (e.g., Alistipes, Prevotella, Paraprevotella, Parabacteroides, or Odoribacter), Clostridium, Streptococcus, Lactococcus, Eubacterium rectale, Escherichia coli, Enterobacter sp., Klebsiella sp., Bifidobacterium, Staphylococcus, Lactobacillus, Veillonella, Haemophilus, Moraxella, Corynebacterium and Propionibacterium. In some embodiments, the bacteria cell is derived from Escherichia coli. In some embodiments, a detectable portion of the administered bacteria cells stably colonize the tissue or surface to which they are administered for at least or at least about 2, 3, 4, 5, 6, 7 days, e.g., at least or at least about 1 week, e.g., at least or at least about 2, 3, 4, 5, 10, 15, 20, 25, 30, 35, 40, 45, 50, 55, 60, 75, 100, 125 weeks, or longer, e.g., for the duration of the life of the subject or for a period that is within a range defined by any two of the aforementioned time periods. In some embodiments, a detectable portion of the administered bacteria cells stably and permanently colonize the tissue or surface to which they are administered. In some embodiments, at least or at least about 10%, 15%, 20%, 25%, 30%, 35%, 40%, 45%, 50%, 55%, 60%, 65%, 70%, 75%, 80%, 85%, 90%, 95% of the administered bacteria cells stably colonize the tissue or surface to which they are administered. In some embodiments, the native/commensal host cells: (i) are capable of metabolizing one or more carbohydrates selected from the group consisting of sucrose, xylose, d-maltose, N-acetyl-d-glucosamine, d-galactose, and d-ribose; (ii) utilize both glycolytic and gluconeogenic substrates; (iii) are non-motile (e.g, have non-functioning flagella, e.g., due to a mutation in the flhDC operon); (iv) are capable of producing ribose-5-phosphate; (v) are able to grow in defined medium lacking vitamin B12 (cyanocobalamin) (e.g., are demonstrated vitamin B12 prototrophs); (vi) express an UDP-glucose-4-epimerase and/or a glycosyltransferase; (vii) comprise multiple copies of a gene encoding β subunit of tryptophan synthase gene; (viii) comprise multiple copies of a gene encoding propionate CoA-transferase; (ix) express capsular polysaccharide (CPS) 4 (CPS4); (x) express an rnf-like oxidoreductase complex; (xi) catabolize tryptophan to yield indole and other indole metabolites, e.g., indole-3-propionate and indole-3-aldehyde; and/or do not produce any agent that induces double-stranded DNA breaks, e.g., do not have a genomic island that encodes giant modular nonribosomal peptide and polyketide synthases, do not express hybrid peptide-polyketide genotoxins, and/or do not have an active clbA gene. In some embodiments, the subject is a human. In some embodiments, at least or at least about 10⁶, 10⁷, 10⁸, 10⁹, 10¹⁰, 10¹¹, 10¹², 10¹³ bacterial cells are administered. In some embodiments, the donating subject and the receiving subject are the same individual, e.g., the microbiome sample is autologous to the subject. In some embodiments, the donating subject and the receiving subject are different individuals. In some embodiments the microbiome sample is from a mammal of the same species as the subject. In some embodiments, the administered bacteria cells are administered to the same tissue or surface from which the microbiome sample was obtained. In some embodiments, the microbiome sample is obtained from the skin or the eye, and the population of bacteria cells is topically administered to the subject, e.g., in a buffered suspension, a gel, a lotion, a cream, or an ointment. In some embodiments, the microbiome sample is obtained from the nasal cavity, and the administered bacteria cells are administered via nasal gavage. In some embodiments, the microbiome sample is obtained from the vagina, and the administered bacteria cells are administered intravaginally. In some embodiments, the microbiome sample is obtained from the GI tract, and the administered bacteria cells are administered to the subject orally or rectally. In some embodiments, the administered bacteria cells are administered orally to the subject via a gastric tube or in an edible composition. In some embodiments, the edible composition comprises a gel capsule comprising the administered bacteria cells or the administered bacteria cells are encapsulated. In some embodiments, the edible composition is selected from the group consisting of yogurt, milk, ice cream, vegetable puree, fruit puree, sorbet, and oatmeal. In some embodiments, the edible composition is a beverage. In some embodiments, the beverage is a buffered solution. In some embodiments, the administered bacteria cells are administered to the subject multiple times, e.g., in daily, weekly, bi-weekly or monthly intervals. In some embodiments, the administered bacteria cells are administered to the subject in daily, weekly, bi-weekly or monthly intervals. In some embodiments, administration of the transformed bacterial cells does not alter the microbiome of the receiving subject.

In a further aspect, provided is a substantially homogeneous population of bacteria cells commensal to a mammal, wherein the population of bacteria is transformed to express one or more polynucleotides that are heterologous to the mammal and/or the bacteria. In some embodiments, the population of bacteria native or commensal to the mammal is capable of stably colonizing permanently or long-term in or on the mammal, e.g., for at least or at least about 2, 3, 4, 5, 6, 7 days, e.g., at least or at least about 1 week, e.g., at least or at least about 2, 3, 4, 5, 10, 15, 20, 25, 30, 35, 40, 45, 50, 55, 60, 75, 100, 125 weeks, or longer, e.g., for the duration of the life of the mammal. In some embodiments, the native/commensal host cells: (i) are capable of metabolizing one or more carbohydrates selected from the group consisting of sucrose, xylose, d-maltose, N-acetyl-d-glucosamine, d-galactose, and d-ribose; (ii) utilize both glycolytic and gluconeogenic substrates; (iii) are non-motile (e.g, have non-functioning flagella, e.g., due to a mutation in the flhDC operon); (iv) are capable of producing ribose-5-phosphate; (v) are able to grow in defined medium lacking vitamin B12 (cyanocobalamin) (e.g., are demonstrated vitamin B12 prototrophs); (vi) express an UDP-glucose-4-epimerase and/or a glycosyltransferase; (vii) comprise multiple copies of a gene encoding β subunit of tryptophan synthase gene; (viii) comprise multiple copies of a gene encoding propionate CoA-transferase; (ix) express capsular polysaccharide (CPS) 4 (CPS4); (x) express an rnf-like oxidoreductase complex; (xi) catabolize tryptophan to yield indole and other indole metabolites, e.g., indole-3-propionate and indole-3-aldehyde; and/or do not produce any agent that induces double-stranded DNA breaks, e.g., do not have a genomic island that encodes giant modular nonribosomal peptide and polyketide synthases, do not express hybrid peptide-polyketide genotoxins, and/or do not have an active clbA gene. In some embodiments, the population of bacterial cells do not comprise one or more polynucleotides encoding for one or more pathogenic toxins selected from the group consisting of AB toxin, Alpha toxin, Anthrax toxin, Botulinum toxin, Cereulide, Cholesterol-dependent cytolysin, Clostridial Cytotoxin family, Clostridium botulinum C3 toxin, Clostridium difficile toxin A, Clostridium difficile toxin B, Clostridium enterotoxin, Clostridium perfringens alpha toxin, Clostridium perfringens beta toxin, Cry1Ac, Cry6Aa, Cry34Ab1, Delta endotoxin, Diphtheria toxin, Enterotoxins, Enterotoxin type B, Erythrogenic toxin, Exfoliatin, Fragilysin, Haemolysin E, Heat-labile enterotoxin, Heat-stable enterotoxin, Hemolysin, HrpZ Family, Leukocidin, Listeriolysin O, Panton-Valentine leucocidin, intact Pathogenicity island, Phenol-soluble modulin, Pneumolysin, Pore-forming toxin, Pseudomonas exotoxin, Pyocyanin, anti-eukaryotic Rhs toxins, RTX toxin, Shiga toxins, Shiga-like toxin, Staphylococcus aureus alpha toxin, Staphylococcus aureus beta toxin, Staphylococcus aureus delta toxin, Streptolysin, Tetanolysin, Tetanospasmin, Toxic shock syndrome toxin, Tracheal cytotoxin, and/or Verocytotoxin. In some embodiments, the population of bacterial cells is antibiotic resistant to one or more antibiotic agents used for selection of the transformed bacterial cells, e.g., kanamycin, chloramphenicol, carbenicillin, hygromycin and/or trimethoprim. In some embodiments, the bacterial cell is not antibiotic resistant to clinically used antibiotic agents. In some embodiments, the bacterial cell is not antibiotic resistant to one or more clinically used antibiotic agents selected from antibiotic macrolides (e.g., azithromycin, clarithromycin, erythromycin, fidaxomicin, telithromycin, carbomycin A, josamycin, kitasamycin, midecamycin/midecamycin acetate, oleandomycin, solithromycin, spiramycin, troleandomycin, tylosin/tylocine, roxithromycin), rifamycins (e.g., rifampicin (or rifampin), rifabutin, rifapentine, rifalazil, rifaximin), polymyxins (e.g., polymyxin B, polymyxin E (colistin)), quinolone antibiotics (e.g., nalidixic acid, ofloxacin, levofloxacin, ciprofloxacin, norfloxacin, enoxacin, lomefloxacin, grepafloxacin, trovafloxacin, sparfloxacin, temafloxacin, moxifloxacin, gatifloxacin, gemifloxacin), beta-lactams (e.g., penicillin, cloxacillin, dicloxacillin, flucloxacillin, methicillin, nafcillin, oxacillin, temocillin, amoxicillin, ampicillin, mecillinam, carbenicillin, ticarcillin, azlocillin, mezlocillin, piperacillin), aminoglycosides (e.g., amikacin, gentamicin, neomycin, streptomycin, tobramycin), cephalosporins (e.g., cefadroxil, cefazolin, cephalexin, cefaclor, cefoxitin, cefprozil, cefuroxime, loracarbef, cefixime, cefdinir, cefditoren, cefoperazone, cefotaxime, cefpodoxime, cefepime, ceftobiprole), monobactams (e.g., aztreonam, tigemonam, nocardicin A, tabtoxinine β-lactam), carbapenems (e.g., biapenem, doripenem, ertapenem, faropenem, imipenem, meropenem, panipenem, razupenem, tebipenem, thienamycin), and tetracyclines (e.g., tetracycline, chlortetracycline, oxytetracycline, demeclocycline, lymecycline, meclocycline, methacycline, minocycline, rolitetracycline, tigecycline). In some embodiments, the one or more heterologous polynucleotides encode a fluorescent protein, e.g., green fluorescent protein, yellow fluorescent protein, red fluorescent protein (mCherry, mEos2, mRuby2, mRuby3, mClover3, mApple, mKate2, mMaple, mCardinal, or mNeptune), mTurquoise, or mVenus. In some embodiments, the one or more heterologous polynucleotides encode an enzyme, a cytokine or a peptide hormone. In some embodiments, the enzyme is a bile salt hydrolase, e.g., from Lactobacillus, e.g., bshA (Gene ID 3251811) or bshB (Gene ID 3252955), N acylphosphatidylethanolamine (NAPE)-hydrolyzing phospholipase D, Actinobacillus actinomycetemcomitans dispersin B (DspB), lactase (beta-galactosidase), an aldehyde dehydrogenase, an alcohol dehydrogenase (e.g., ADH1A, ADH1B, ADH1C, ADH2, ADH3, ADH4, ADH5, ADH6, ADH7), bile acid-CoA:amino acid N-acyltransferase (BAAT), phenylalanine hydroxylase, butyrate synthesis pathway enzymes, Aspergillus niger-derived prolyl endoprotease (AN-PEP), 7 alpha-hydroxysteroid dehydrogenase (7-alpha-HSDH), 7 beta-hydroxysteroid dehydrogenase (7-beta-HSDH), cholylglycine hydrolase and cholic acid 7alpha-dehydroxylase. In some embodiments, the cytokine is selected from the group consisting of mammalian (e.g., human) IL-10 and mammalian (e.g., human) IL-27 dimer (IL27 alpha subunit and Epstein-Barr virus induced 3 (EBI3) subunit expressed separately or as a fusion protein), and TGF-β. In some embodiments, the peptide hormone is selected from the group consisting of mammalian glucagon, glucagon-like peptide 1 (GLP-1), mammalian glucagon-like peptide 2 (GLP-2), Fibroblast growth factor 1 (FGF1), Fibroblast growth factor 15 (FGF15), Fibroblast growth factor 19 (FGF19), insulin, and proinsulin. In some embodiments, the one or more heterologous polynucleotides encode Akkermansia muciniphila Amuc_1100, Vibrio vulnificus flagellin B, elafin, trefoil factor 1 (TFF1), trefoil factor 2 (TFF2), trefoil factor 3 (TFF3), anti-TNFα antibodies/nanobodies or fragments or single chains thereof, Nostoc elipsosporum cyanovirin-N or microcin J25 (MccJ25). In some embodiments, the one or more heterologous polynucleotides comprise codon bias and/or codon optimization configured to improve or enhance expression of the heterologous protein in the transformed population of the isolated and cultured bacterial cells. In some embodiments, the one or more heterologous polynucleotides are integrated into the chromosome of the bacterial cells of the transformed population. In some embodiments, the one or more heterologous polynucleotides are integrated into the attB and/or yfgG genes of the bacterial genome. In some embodiments, the heterologous polynucleotide is in a plasmid episomally located in the bacterial cells. In some embodiments, the transformed bacterial cells further comprise a plasmid retention or maintenance system, e.g., a partitioning system or a toxin-antitoxin module or system. In some embodiments, the one or more heterologous polynucleotides are integrated into an expression cassette having at least or at least about 80%, 85%, 90%, 95%, 97%, 99% or 100% sequence identity to SEQ ID NO:2, and are expressed under the control of a Ptrc promoter. In some embodiments, the substantially homogenous population of bacterial cells is from a gram negative bacterial strain. In some embodiments, the substantially homogenous population of bacterial cells is derived from a bacteria genus selected from the group consisting of Bacteroides (e.g., Alistipes, Prevotella, Paraprevotella, Parabacteroides, or Odoribacter), Clostridium, Streptococcus, Lactococcus, Eubacterium rectale, Escherichia coli, Enterobacter sp., Klebsiella sp., Bifidobacterium, Staphylococcus, Lactobacillus, Veillonella, Haemophilus, Moraxella, Corynebacterium and Propionibacterium. In some embodiments, the substantially homogenous population of bacterial cells is derived from Escherichia coli. In some embodiments, the population of bacteria cells is lyophilized or cryopreserved.

In another aspect, provided are pharmaceutical compositions suitable for administration to a mammal, e.g., for delivery of one or more therapeutic polypeptides to the mammal. In another aspect, provided are edible compositions. In some embodiments, the compositions comprises a substantially homogeneous population of bacteria cells commensal to the mammal, wherein the population of bacteria is transformed to express one or more polynucleotides that are heterologous to the mammal and/or the bacteria, as described above and herein. In some embodiments, the population of bacteria native or commensal to the mammal is produced according to methods described above and herein. In some embodiments, the population of bacteria commensal to the mammal is capable of colonizing or is configured to colonize in or on the mammal permanently or long-term, e.g., for at least or at least about 2, 3, 4, 5, 6, 7 days, e.g., at least or at least about 1 week, e.g., at least or at least about 2, 3, 4, 5, 10, 15, 20, 25, 30, 35, 40, 45, 50, 55, 60, 75, 100, 125 weeks, or longer, e.g., for the duration of the life of the mammal. In some embodiments, the native/commensal host cells: (i) are capable of metabolizing one or more carbohydrates selected from the group consisting of sucrose, xylose, d-maltose, N-acetyl-d-glucosamine, d-galactose, and d-ribose; (ii) utilize both glycolytic and gluconeogenic substrates; (iii) are non-motile (e.g, have non-functioning flagella, e.g., due to a mutation in the flhDC operon); (iv) are capable of producing ribose-5-phosphate; (v) are able to grow in defined medium lacking vitamin B12 (cyanocobalamin) (e.g., are demonstrated vitamin B12 prototrophs); (vi) express an UDP-glucose-4-epimerase and/or a glycosyltransferase; (vii) comprise multiple copies of a gene encoding β subunit of tryptophan synthase gene; (viii) comprise multiple copies of a gene encoding propionate CoA-transferase; (ix) express capsular polysaccharide (CPS) 4 (CPS4); (x) express an rnf-like oxidoreductase complex; (xi) catabolize tryptophan to yield indole and other indole metabolites, e.g., indole-3-propionate and indole-3-aldehyde; and/or do not produce any agent that induces double-stranded DNA breaks, e.g., do not have a genomic island that encodes giant modular nonribosomal peptide and polyketide synthases, do not express hybrid peptide-polyketide genotoxins, and/or do not have an active clbA gene. In some embodiments, the population of bacterial cells do not comprise one or more polynucleotides encoding for one or more pathogenic toxins selected from the group consisting of AB toxin, Alpha toxin, Anthrax toxin, Botulinum toxin, Cereulide, Cholesterol-dependent cytolysin, Clostridial Cytotoxin family, Clostridium botulinum C3 toxin, Clostridium difficile toxin A, Clostridium difficile toxin B, Clostridium enterotoxin, Clostridium perfringens alpha toxin, Clostridium perfringens beta toxin, Cry1Ac, Cry6Aa, Cry34Ab1, Delta endotoxin, Diphtheria toxin, Enterotoxins, Enterotoxin type B, Erythrogenic toxin, Exfoliatin, Fragilysin, Haemolysin E, Heat-labile enterotoxin, Heat-stable enterotoxin, Hemolysin, HrpZ Family, Leukocidin, Listeriolysin O, Panton-Valentine leucocidin, intact Pathogenicity island, Phenol-soluble modulin, Pneumolysin, Pore-forming toxin, Pseudomonas exotoxin, Pyocyanin, anti-eukaryotic Rhs toxins, RTX toxin, Shiga toxins, Shiga-like toxin, Staphylococcus aureus alpha toxin, Staphylococcus aureus beta toxin, Staphylococcus aureus delta toxin, Streptolysin, Tetanolysin, Tetanospasmin, Toxic shock syndrome toxin, Tracheal cytotoxin, and/or Verocytotoxin. In some embodiments, the population of bacterial cells is antibiotic resistant to one or more antibiotic agents used for selection of the transformed bacterial cells, e.g., kanamycin, chloramphenicol, carbenicillin, hygromycin and/or trimethoprim. In some embodiments, the bacterial cell or population of bacterial cells is not antibiotic resistant to clinically used antibiotic agents. In some embodiments, the bacterial cell or populations of bacterial cells is not antibiotic resistant to one or more clinically used antibiotic agents selected from antibiotic macrolides (e.g., azithromycin, clarithromycin, erythromycin, fidaxomicin, telithromycin, carbomycin A, josamycin, kitasamycin, midecamycin/midecamycin acetate, oleandomycin, solithromycin, spiramycin, troleandomycin, tylosin/tylocine, roxithromycin), rifamycins (e.g., rifampicin (or rifampin), rifabutin, rifapentine, rifalazil, rifaximin), polymyxins (e.g., polymyxin B, polymyxin E (colistin)), quinolone antibiotics (e.g., nalidixic acid, ofloxacin, levofloxacin, ciprofloxacin, norfloxacin, enoxacin, lomefloxacin, grepafloxacin, trovafloxacin, sparfloxacin, temafloxacin, moxifloxacin, gatifloxacin, gemifloxacin), beta-lactams (e.g., penicillin, cloxacillin, dicloxacillin, flucloxacillin, methicillin, nafcillin, oxacillin, temocillin, amoxicillin, ampicillin, mecillinam, carbenicillin, ticarcillin, azlocillin, mezlocillin, piperacillin), aminoglycosides (e.g., amikacin, gentamicin, neomycin, streptomycin, tobramycin), cephalosporins (e.g., cefadroxil, cefazolin, cephalexin, cefaclor, cefoxitin, cefprozil, cefuroxime, loracarbef, cefixime, cefdinir, cefditoren, cefoperazone, cefotaxime, cefpodoxime, cefepime, ceftobiprole), monobactams (e.g., aztreonam, tigemonam, nocardicin A, tabtoxinine β-lactam), carbapenems (e.g., biapenem, doripenem, ertapenem, faropenem, imipenem, meropenem, panipenem, razupenem, tebipenem, thienamycin), and tetracyclines (e.g., tetracycline, chlortetracycline, oxytetracycline, demeclocycline, lymecycline, meclocycline, methacycline, minocycline, rolitetracycline, tigecycline). In some embodiments, the one or more heterologous polynucleotides encode a fluorescent protein, e.g., green fluorescent protein, yellow fluorescent protein, red fluorescent protein (mCherry, mEos2, mRuby2, mRuby3, mClover3, mApple, mKate2, mMaple, mCardinal, or mNeptune), mTurquoise, or mVenus. In some embodiments, the one or more heterologous polynucleotides encode an enzyme, a cytokine or a peptide hormone. In some embodiments, the enzyme is a bile salt hydrolase, e.g., from Lactobacillus, e.g., bshA (Gene ID 3251811) or bshB (Gene ID 3252955), N acylphosphatidylethanolamine (NAPE)-hydrolyzing phospholipase D, Actinobacillus actinomycetemcomitans dispersin B (DspB), lactase (beta-galactosidase), an aldehyde dehydrogenase, an alcohol dehydrogenase (e.g., ADH1A, ADH1B, ADH1C, ADH2, ADH3, ADH4, ADH5, ADH6, ADH7), bile acid-CoA:amino acid N-acyltransferase (BAAT), phenylalanine hydroxylase, butyrate synthesis pathway enzymes, Aspergillus niger-derived prolyl endoprotease (AN-PEP), 7 alpha-hydroxysteroid dehydrogenase (7-alpha-HSDH), 7 beta-hydroxysteroid dehydrogenase (7-beta-HSDH), cholylglycine hydrolase and cholic acid 7alpha-dehydroxylase. In some embodiments, the cytokine is selected from the group consisting of mammalian (e.g., human) IL-10 and mammalian (e.g., human) IL-27 dimer (IL27 alpha subunit and Epstein-Barr virus induced 3 (EBI3) subunit expressed separately or as a fusion protein), and TGF-β. In some embodiments, the peptide hormone is selected from the group consisting of glucagon, mammalian glucagon-like peptide 1 (GLP-1), mammalian glucagon-like peptide 2 (GLP-2), Fibroblast growth factor 1 (FGF1), Fibroblast growth factor 15 (FGF15), Fibroblast growth factor 19 (FGF19), insulin, and proinsulin. In some embodiments, the one or more heterologous polynucleotides encode Akkermansia muciniphila Amuc_1100, Vibrio vulnificus flagellin B, elafin, trefoil factor 1 (TFF1), trefoil factor 2 (TFF2), trefoil factor 3 (TFF3), anti-TNFα antibodies/nanobodies or fragments or single chains thereof, Nostoc elipsosporum cyanovirin-N or microcin J25 (MccJ25). In some embodiments, the one or more heterologous polynucleotides comprise codon bias and/or codon optimization configured to improve or enhance expression of the heterologous protein in the transformed population of the isolated and cultured bacterial cells. In some embodiments, the one or more heterologous polynucleotides are integrated into the chromosome of the bacterial cells of the transformed population. In some embodiments, the one or more heterologous polynucleotides are integrated into the attB and/or yfgG genes of the bacterial genome. In some embodiments, the heterologous polynucleotide is in a plasmid episomally located in the bacterial cells. In some embodiments, the transformed bacterial cells further comprise a plasmid retention or maintenance system, e.g., a partitioning system or a toxin-antitoxin module or system. In some embodiments the one or more heterologous polynucleotides are integrated into an expression cassette having at least or at least about 80%, 85%, 90%, 95%, 97%, 99% or 100% sequence identity to SEQ ID NO:2, and are expressed under the control of a Ptrc promoter. In some embodiments, the substantially homogenous population of bacterial cells is from a gram negative bacterial strain. In some embodiments, the substantially homogenous population of bacterial cells is derived from a bacteria genus selected from the group consisting of Bacteroides (e.g., Alistipes, Prevotella, Paraprevotella, Parabacteroides, or Odoribacter), Clostridium, Streptococcus, Lactococcus, Eubacterium rectale, Escherichia coli, Enterobacter sp., Klebsiella sp., Bifidobacterium, Staphylococcus, Lactobacillus, Veillonella, Haemophilus, Moraxella, Corynebacterium and Propionibacterium. In some embodiments, the substantially homogenous population of bacterial cells is derived from Escherichia coli. In some embodiments, the composition comprises a buffered solution or buffered suspension. In some embodiments, the edible composition comprises a gel capsule comprising the administered bacteria cells or the administered bacteria cells are encapsulated. In some embodiments, the edible composition comprises a beverage. In some embodiments, the edible composition is selected from the group consisting of yogurt, milk, ice cream, vegetable puree, fruit puree, sorbet and oatmeal.

In a further aspect, provided are kits comprising one or more containers comprising one or more compositions as described above and herein. In some embodiments, the population of bacterial cells are lyophilized.

### Definitions

The terms “commensal bacteria” or “native bacteria” interchangeably refer to a bacteria cell or population of cells obtained from, and adapted to, or configured for the microbiome of a mammal. Commensal bacterial are adapted to colonize or configured for colonization of a mammal (e.g., bodily excretions (e.g. saliva, mucus, urine, or stool), surfaces (e.g. mucosal GI tract, mouth/pharynx/nares, urogenital track, skin, anus/rectum, cheek/mouth, or eye), and are not adapted for or configured for culture in a laboratory environment.

As used herein, “administering” refers to local and systemic administration, e.g., including enteral, parenteral, pulmonary, and topical/transdermal administration. Routes of administration for engineered native bacteria (ENB) that find use in the methods described herein include, e.g., oral (per os (P.O.)), rectal (e.g., administration as a suppository), vaginal, nasal or inhalation, topical contact (e.g., to skin or eyes), or intralesional administration to a subject. Administration can be by any route including parenteral and/or transmucosal (e.g., oral, nasal, vaginal, or rectal). Administering can be performed by a health worker or can include self-administration.

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, and/or rectal administration.

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

The term “co-administering” or “concurrent administration”, refers to administration of the multiple ENB populations or one or more ENB populations with another active agent such that both can simultaneously achieve a physiological effect. The two agents, however, need not be administered together. In certain embodiments, administration of one agent can precede administration of the other. Simultaneous physiological effect need not necessarily require presence of both agents in the circulation at the same time. However, in certain embodiments, co-administering typically results in both agents being simultaneously present in the body (e.g., in the plasma) at a significant fraction (e.g., 20% or greater, preferably 30% or 40% or greater, more preferably 50% or 60% or greater, most preferably 70% or 80% or 90% or greater) of their maximum serum concentration for any given dose.

The term “effective amount” or “pharmaceutically effective amount” refer to the amount and/or dosage, and/or dosage regime of one or more compounds necessary to bring about the desired result e.g., an amount sufficient to mitigate in a mammal one or more symptoms associated with the disease condition for which the subject is receiving therapy, or an amount sufficient to lessen the severity or delay the progression of the disease condition in a mammal (e.g., therapeutically effective amounts), an amount sufficient to reduce the risk or delaying the onset, and/or reduce the ultimate severity of a disease condition in a mammal (e.g., prophylactically effective amounts).

As used herein, the terms “treating” and “treatment” refer to delaying the onset of, retarding or reversing the progress of, reducing the severity of, or alleviating or preventing either the disease or condition to which the term applies, or one or more symptoms of such disease or condition.

The term “mitigating” refers to a reduction or elimination of one or more symptoms of that pathology or disease, and/or a reduction in the rate or delay of onset or severity of one or more symptoms of that pathology or disease, and/or the prevention of that pathology or disease.

The terms “subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig) and/or agricultural mammals (e.g., equine, bovine, porcine, or ovine). In various embodiments, the subject can be a human (e.g., adult male, adult female, adolescent male, adolescent female, male child, or female child) under the care of a physician or other healthworker in a hospital, psychiatric care facility, as an outpatient, or other clinical context. In certain embodiments the subject may not be under the care or prescription of a physician or other healthworker.

As used herein, a “substantially homogenous population of bacteria cells” is genetically at least 90%, 91%, 92%, 93%, 94%, 95%, 96%, 97%, 98%, 99% identical, e.g., as determined by whole genome sequencing or by sequencing the ribosomal RNA 16S DNA.

As used herein, engineered native bacterial (ENB) cells that “stably colonize” establish themselves and divide (e.g., multiply) at or in the vicinity of the lumen or tissue to which they have been administered such that they remain, e.g. at least 3, 4, 5 or 6 days, e.g., at least 1, 2, 3, 4, 5, 10, 15, 20, 25, 30, 35, 40, 45, 50, 55, 60, 75, 100, 125 weeks, or longer, e.g., for the duration of the life of the subject or for a time period that is within a range defined by any two of the aforementioned time periods.

The term “heterologous nucleic acid” or “heterologous polypeptide” refers to a nucleic acid or a polypeptide whose sequence is not identical to that of another nucleic acid or polypeptide naturally found in the same host cell or the same host. As use herein, the “heterologous nucleic acid” or “heterologous polypeptide” can be heterologous to the bacterial cell and/or the mammalian host.

As used herein, the term “transform” or “transformation” refers to the transfer of a nucleic acid fragment into a host bacterial cell, resulting in genetically-stable inheritance. Host bacterial cells comprising the transformed nucleic acid fragment are referred to as “recombinant” or “transgenic” or “transformed” organisms.

The term “therapeutic polypeptide” refers to a polypeptide having therapeutic pharmacological activity in a mammal.

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least or at least about 80% identity, for example, at least or at least about 85%, 90%, 91%, 92%, 93%, 94%, 95%, 96%, 97%, 98%, 99% or 100% identity over a specified region to a reference sequence, e.g., heterologous polynucleotide or polypeptide sequences as described in Table 1, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least or at least about 25 amino acids or nucleotides in length, for example, over a region that is 50, 100, 200, 300, 400 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to reference nucleic acids and proteins, the BLAST and BLAST 2.0 algorithms and the default parameters are used.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

### BRIEF DESCRIPTION OF THE DRAWINGS

FIG. 1 illustrates a schematic overview of the methods described herein. Native (i.e., commensal) bacteria can be used as vectors to introduce new functions (e.g., delivery of a therapeutic polypeptide, e.g., a bile salt hydrolase) to the microbiome (e.g., gut, skin) of conventionally raised, wild-type hosts (such as humans).

FIG. 2 illustrates a schematic of how engineered bacteria expressing BSH could affect neuroinflammation and cognition. Bile acids (BAs) are key mediators of the microbiome-gut-brain axis. Luminal BA deconjugation affects neuroinflammation and cognitive performance in diet-induced obesity mice.

FIGS. 3A-C. A. Action of bile salt hydrolase (BSH) on taurocholic acid. BSH deconjugates bile acids, such as converting taurocholic acid (TCA) to cholic acid (CA). This action makes bile acids more difficult to reabsorb and allows them to be further processed by other bacteria in the gut microbiome. B. The native E. coli (here identified as ENB) can be genetically modified to express BSH as shown by in vitro deconjugation of TCA to CA with cells in PBS buffer. C. Native E. coli (top left) can be engineered to express GFP (top right) and further engineered to express bile salt hydrolase (bottom) which can deconjugate TDCA (soluble) to DCA (insoluble—white halo).

FIGS. 4A-F. A. Colonization of mice with genetically engineered derivatives of strain AZ-39. The engineered native E. coli remains at a stable level for more than a year after a single gavage (n=12-16 to 100 days; n=3-8 to 300+ days) in different dietary conditions (e.g. both normal chow diet and high fat diet feeding conditions). B. Engineered native bacteria colonize the entire gut, especially distal gut. The genetically engineered native E. coli colonizes the entire gut, and is mostly concentrated in terminal ileum and cecum (n=4-6). C. Engineered native bacteria do not affect normal weight gain or diet-induced obesity in mice. Colonization with the genetically engineered native E. coli did not affect mouse body weight in both the high fat diet and normal chow diet condition (n=12-16). Note that previous reports of expression of this gene in immune-compromised and germ-free mice suggested that BSH would affect adiposity, but it does not do so in a conventionally-raised wild-type mouse. D-E. Summary graphs of FIG. 4F. Colonization of germ-free mice with the recombineered native bacteria expressing GFP did not affect fecal bile acid composition. However, the recombineered native bacteria expressing both GFP and BSH showed much higher levels of deconjugated bile acids (n=3). F. Engineered Native E. coli expressing BSH deconjugate bile acids in germ-free mice (blue: non-colonized mice; green: mice colonized with AZ-39 expressing GFP; red: mice colonized with AZ-39 expressing GFP and BSH). The engineered native bacteria expressing both GFP and BSH (red) showed much higher levels of deconjugated bile acids, especially of TCA and TbMCA (n=3). There was no significant difference in the other unconjugated/conjugated bile acids.

FIGS. 5A-C. A. Engineered Native Bacteria do not change the composition of the gut microbiome. The addition of recombineered native bacteria (regardless of whether it includes BSH or not) does not change the gut microbiome in a way that is detectable by 16S sequencing and analysis (top panel shows, weighted PCoA, unweighted PCoA, and Bray-Curtis distance) 10 weeks after colonization. However, in correlational analysis, certain OTUs related to Bacteroides became highly correlated to the E. coli bacteria expressing BSH suggesting that even though overall composition doesn't change, there are changes in the relationships between the bacteria within the gut. B-C. Engineered Native Bacteria affect both fecal and serum bile acid pools. Fecal (top) and serum (bottom) bile acid correlograms. The addition of native bacteria with bile salt hydrolase caused subtle changes in the fecal bile acids (see FIG. 6 for examples). However, the native bacteria with BSH caused major changes in the serum bile acids. In these mice, the serum BAs showed anti-correlation between conjugated and deconjugated BAs.

FIGS. 6A-B. A. Engineered Native Bacteria affect fecal bile acids. Examples of fecal bile acids that were affected by our engineered native bacteria. Tauro-beta-muricholic acid (TbMCA; left) and TCA (right) were much lower in the mice that had received the native bacteria recombineered to express BSH. TDCA (middle) was much higher in these mice. DCA is a secondary bile acid, and microbial synthesis of DCA requires deconjugation as the first step. B. Engineered Native Bacteria affect serum bile acids. There were more dramatic differences in the serum bile acids than in stool bile acids. Beta-muricholic acid (bMCA), tauro-bMCA, and omega-muricholic acid (oMCA) levels were reduced in mice that had received the engineered bacteria expressing BSH.

FIGS. 7A-D. A. An engineered native bacteria (ENB) expressing BSH alter overall metabolism: measurement of respiratory exchange ratio (V_{CO2}/V_{O2}; RER) in engineered bacteria expressing and not expressing BSH. Mice colonized with engineered native bacteria expressing BSH had a significantly lower RER than mice colonized with engineered native bacteria not expressing BSH. This suggests that these mice preferentially use more fatty acids for their metabolism rather than carbohydrates. B. Engineered native bacteria can alter host physiology. The mice which had received the native bacteria that expressing BSH had normal fasting insulin levels (left). However, their post-prandial insulin levels (right) were significantly lower than the control cohorts suggesting higher insulin sensitivity. C. Engineered native bacteria expressing BSH affect behavior. Mice containing engineered native bacteria expressing BSH (red) exercised nearly 50% more than the mice that did not have this bacteria (green) or had the bacteria without the BSH gene (blue). D. Engineered native bacteria expressing BSH affect cognition. In a novel object recognition test, all the mice on a normal chow diet, regardless of whether they were colonized by engineered bacteria or not, spent more time on the novel object. However, as seen in previous studies, mice maintained on a high fat diet could not distinguish between the novel object and the old object, suggesting they had problems with their memory. Mice who had received the engineered native bacteria expressing BSH, however, appear to have normal increased interest in the novel object, and thus rescued memory function.

FIG. 8 illustrates that by administering native bacteria engineered to express BSH, we affected host physiology. The mice, which had received the engineered native bacteria expressing BSH, had lower fasting glucagon levels.

FIGS. 9A-B. A. Oral glucose tolerance test of wild-type conventionally-raised mice on a normal chow diet colonized with engineered native bacteria. Though engineered native bacteria expressing BSH caused lower post-prandial insulin, there was no difference in serum glucose levels regardless of whether or not the bacteria expressed BSH. B. Oral glucose tolerance test of ob/ob conventionally-raised mice on a normal chow diet colonized with engineered native bacteria (blue: AZ51/BSH−, orange: AZ52/BSH+). In this animal model of obesity/type 2 diabetes, expression of BSH by the engineered bacteria significantly improved insulin sensitivity.

FIG. 10 illustrates engineered native bacteria colonize mice regardless of diet after a single gavage. NFD: no-fat diet, VLFD: very low-fat diet, LFD: low-fat diet.

FIG. 11 illustrates that at 22 weeks post-gavage, engineered native bacteria retain BSH activity.

FIG. 12 illustrates that retention of GFP and BSH gene expression X weeks after gavage. Only a single isolate which was expected to have BSH activity did not have BSH activity (bold).

FIG. 13 illustrates that antibiotic sensitivity of an engineered native E. coli. These strains do not contain genes homologous to beta-lactams. Simultaneous resistance to cephalexin and sensitivity to carbenicillin suggest specific resistance through PBP mutations.

FIG. 14 illustrates that total bile acids in fecal pellets from colonized mice. Presence of the BSH gene (AZ-52) produces significantly greater loss of bile acids in feces than absence of the BSH gene (AZ-51; p<0.003).

FIG. 15 illustrates that the chromosomal yfgG site for transgene incorporation is retained in 208 of 210 complete E. coli genomes (NCBI GenBank nt database). A straight line indicates intact insertion site; a broken, vertically-offset line indicates chromosomal rearrangement at that site.

FIG. 16 illustrates that no bacterial overgrowth is associated with colonization, regardless of BSH presence or absence after 6 months of colonization. Terminal ileum tissue was flash frozen, powderized, and total DNA was extracted. Bacterial 16S copy number and host GAPDH copy number were assessed by quantitative PCR, and the 16S abundance was normalized by host GAPDH copy number.

FIG. 17 illustrates that engineered native bacteria do not significantly alter the terminal ileum microbiome, regardless of BSH presence/absence.

FIG. 18 illustrates that engineered native E. coli do not alter host feces production after 2 months of colonization. Colonized mice were singly-housed and feces were collected every 3 hours for 48 hours.

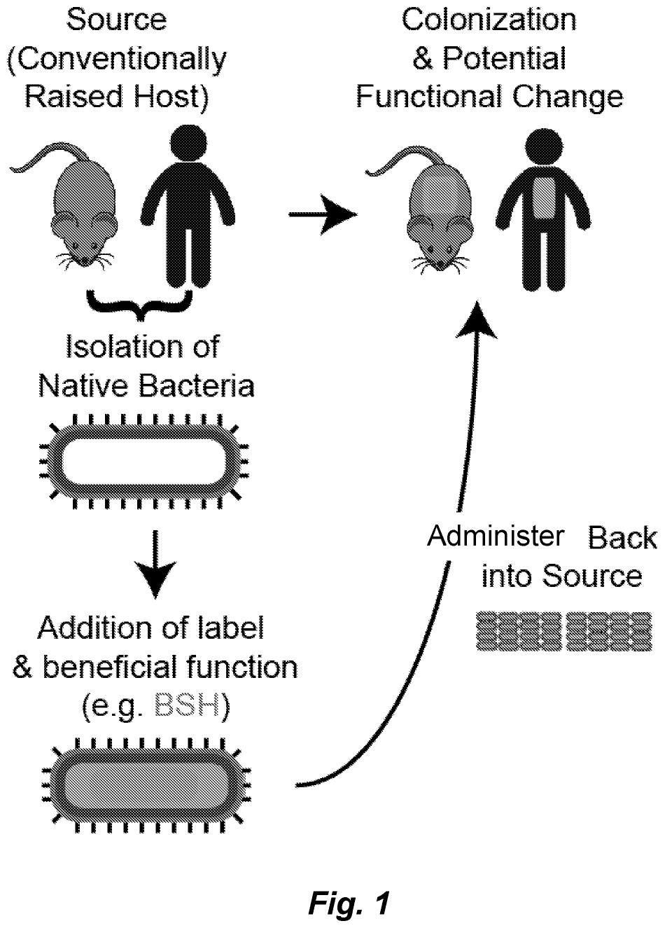

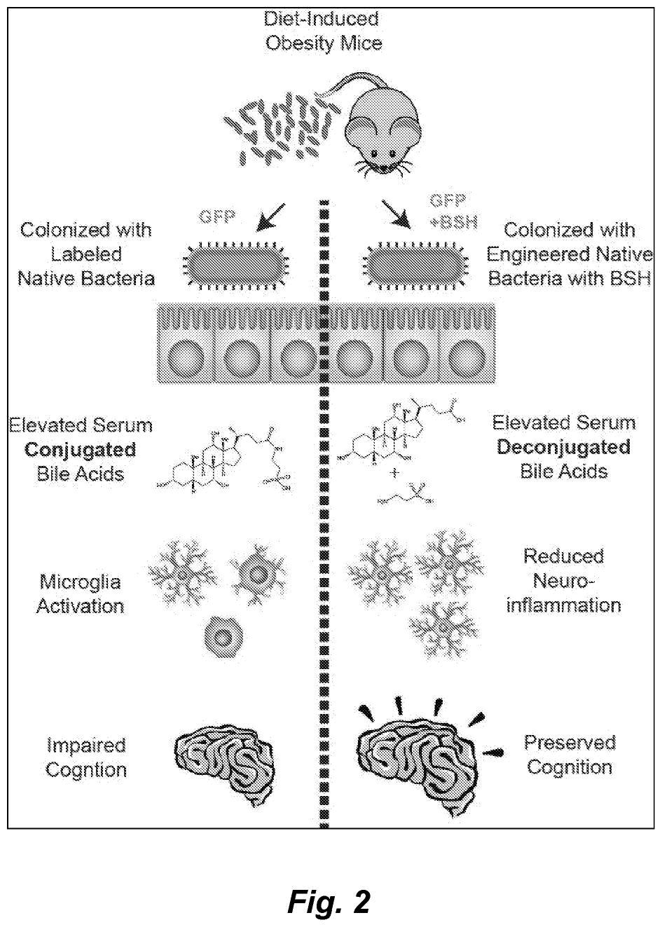

### DETAILED DESCRIPTION

### 1. Introduction

Few tools are available for researchers to functionally manipulate the gut microbiome and to attain a better mechanistic understanding of the host-microbe relationship. We have developed a technique to “knock-in” functions into the gut microbiome to investigate their effects on the luminal ecology, the flux of metabolites and nutrients, and, ultimately, physiology in conventionally-raised, wild-type (CR-WT) mice (e.g., as opposed to mice raised in germ-free environments). We can accomplish this by identifying and engineering tractable native bacteria (as opposed to lab strains or purported commensal bacteria) to express a gene of interest in the luminal environment.

The methods and compositions described herein circumvent the problems of traditional probiotic microorganisms by reengineering commensal organisms to provide a therapeutic function. Commensal strains of microorganisms are a reservoir of organisms which are, by their very definition, capable of stable and long-term or permanent colonization of at least one specific mammalian host. Current probiotics are a single strain, which is used in multiple hosts but have not had great success in broad populations.

Colonizing new hosts using known probiotic microorganisms to alter physiological processes presented challenges. Therefore, we sought to develop a method to colonize the human surfaces (e.g. gastrointestinal tract or skin) with greater reliability. The present methods are based on the discovery of a technique to reliably perform microbiome transplants. Briefly, a commensal bacterial strain isolated from a human subject is cultured and transformed with a heterologous polynucleotide to express a heterologous protein to render a therapeutic effect, and then administered to the same or a different human subject in the engineered form (e.g., an autologous or allogeneic microbiome transplant). Herein, we demonstrate that long-term colonization and effective functional change in the gut microbiome can be accomplished by using native bacteria derived from the host as vectors to introduce new genes and functions to the luminal environment. The reluctance to use this method in the past was driven by the assumption that it is difficult to culture and modify native bacteria. By using native bacteria instead of lab strains, we are employing host cells that are already adapted to the host's luminal environment. This allows engineered native bacteria (ENB) to colonize and induce a functional change in CR-WT hosts.

We have been able to identify, culture and isolate tractable native bacteria derived from the CR-WT host, genetically modifying them with genes theorized to impart a beneficial function, and then re-introducing the ENB to the CR-WT host. Hence, ENBs are already adapted to the luminal environment. In our preliminary studies, we used a native E. coli isolated from mouse feces. Bacterial engineering of E. coli can be performed by practically any lab with very little resources. Though E. coli are common native bacteria, many investigators have assumed that they are not good colonizers since there has been so much disappointment with lab strains. However, using our approach described herein, we have been successful creating engineered bacteria host cells transformed to express heterologous polynucleotides, and that can deliver therapeutic polypeptides to a mammal. This strategy addresses the problem wherein a single specific strain is highly variable in colonizing many different hosts. We demonstrate success in stably colonizing the gastrointestinal tracts of mice with engineered commensal bacterial isolated from the stool of a single mouse.

In our studies in CR-WT mice, we altered luminal bile and serum bile acids using our engineered native bacteria (ENB) to knock in bile salt hydrolase (BSH), a bacterial enzyme that deconjugates luminal bile acids (BAs) and is thought to affect multiple host physiological processes including metabolism. In addition to affecting metabolism, we were surprised to find that activation of BSH in the gut lumen additionally affected behavior and cognition. Previous studies show a link between BAs, neuroinflammation, and cognition, and the results described herein are consistent with the conclusion that BAs are mediators of the microbiome-gut-brain axis.

By knocking in a gene (e.g., bile salt hydrolase (BHS)) into the gut microbiome, we are able to mitigate neuroinflammation. By ameliorating neuroinflammation, we can treat a host of pathophysiological problems, including but not limited to, obesity, type 2 diabetes, traumatic brain injury, dementia, stroke, and certain encephalopathies. Herein, we show that we can improve cognition in mice that have neuroinflammation due to diet-induced obesity.

1. Methods of Preparing Engineered Native/Commensal Bacteria

The methods for preparing therapeutically suitable engineered native or commensal bacteria for transformation with a heterologous polynucleotide are straightforward.

A microbiome sample is obtained from a patient. The microbiome sample can be from any bacterial population stably colonized on a surface or in a lumen of the individual. Microbial communities having stably colonized bacterial communities can be found on all environmentally exposed sites in the body, including the skin, nasopharynx, oral cavity, respiratory tract, gastrointestinal tract and/or female reproductive tract. Accordingly, in some embodiments, the microbiome sample is obtained by swabbing, flushing or taking a biopsy of skin, nasopharynal cavity, or oral cavity (e.g., cheek, tongue, gums, or throat), respiratory tract, gastrointestinal tract and/or the urogenital tract. In some embodiments, the microbiome sample is obtained from a fecal sample. In some embodiments, the microbiome sample is obtained from a tissue biopsy (e.g., obtained during endoscopy, scrape biopsy, or punch biopsy). In some embodiments, the microbiome sample is obtained from a tissue surface (e.g., by swabbing or flushing with a solution). As appropriate, samples can be collected in a series of ways including but not limited to: culture of bodily fluids (e.g. saliva, mucus, urine, stool, or breath), biopsy of a surface (e.g. mucosal biopsies of GI tract, biopsies of mouth/pharynx/nares, or biopsies of urogenital track, biopsies of skin), swabs (e.g. skin, anus/rectum, cheek/mouth, or eye), and/or pathological specimens (e.g. cancerous tissue, amputated limbs, or inflamed organs). A microbiome sample comprises a sufficient number of cells to initiate one or more cultures in vitro for isolation, e.g., at least or at least about 1, 10, 100, 1000, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ bacterial cells.

The microbiome sample is homogenized and bacterial cells from the homogenate are cultured on solid agar substrate to isolate a bacteria cell from which to cultivate a substantially homogenous population of native or commensal bacterial cells for transformation with a heterologous polynucleotide. Employing techniques known in the art, the homogenized sample is streaked on selective or indicative solid microbiological media, depending on the species of commensal or native bacteria to be isolated and cultured. Common bacterial genera found in the human microbiome, and which can be isolated and transformed to express a heterologous polynucleotide include, e.g., Bacteroides, Clostridium, Streptococcus, Lactococcus, Eubacterium rectale, Escherichia coli, Enterobacter sp., Klebsiella sp., Bifidobacterium, Staphylococcus, Lactobacillus, Veillonella, Haemophilus, Moraxella, Corynebacterium and Propionibacterium. Species within Bacteroides, previously considered the most prevalent and abundant bacterial genus in the gut, have been reclassified into five genera: Alistipes, Prevotella, Paraprevotella, Parabacteroides, or Odoribacter (Rajilic-Stojanovic, et al., FEMS Microbiol Rev. 2014; 38:996-1047). As appropriate, MacConkey Lactose Agar or Violet Red Bile Dextrose Agar can be used to isolate and culture E. coli cells. De Man-Rogosa-Sharpe Agar can be used to isolate and culture Lactobacillus spp. cells. Bile Esculin Agar can be used to isolate and culture Enterococcus spp. cells. Wilkins-Chalgren Anaerobe Agar can be used to isolate and culture Bacteroides spp. cells. TPY medium can be used to isolate and culture Bifidobacteria spp. BM9 or GM17c media can be used for Lactococcus spp. Bacterial species commonly colonizing the human microbiome are described, e.g., by the Human Microbiome Project Consortium in Nature. (2012) Jun. 13; 486(7402):207-14 and Lloyd-Price, Genome Med. 2016 Apr. 27; 8(1):51.

Bacteria species commonly found in the human colon and which can be substantially isolated for transformation with a heterologous polynucleotide include, e.g., Bacteroides fragilis, Bacteroides melaninogenicus, Bacteroides oralis, Enterococcus faecalis, Escherichia coli, Enterobacter sp., Klebsiella sp., Bifidobacterium bifidum, Staphylococcus aureus, Lactobacillus, Clostridium perfringens, Proteus mirabilis, Clostridium tetani, Clostridium septicum, Pseudomonas aeruginosa, Salmonella enterica, Faecalibacterium prausnitzii, Peptostreptococcus sp. And/or Peptococcus sp.

Bacteria species commonly found in the human stool and which can be substantially isolated for transformation with a heterologous polynucleotide include, e.g., E. coli, Prevotella copri, Alistipes putredinis and/or Bacteroides vulgatus.

Skin sites are colonized primarily by bacterial genera Corynebacterium, Propionibacterium, and/or Staphylococcus, which can be isolated and transformed to express a heterologous polynucleotide. Bacteria species commonly found on human skin and which can be substantially isolated for transformation with a heterologous polynucleotide include, e.g., Staphylococcus epidermidis, Staphylococcus aureus, Staphylococcus warneri, Streptococcus pyogenes, Streptococcus mitis, Propionibacterium acnes, Corynebacterium spp., Acinetobacter johnsonii, and/or Pseudomonas aeruginosa.

Bacterial species commonly found in the human oral cavity and which can be substantially isolated for transformation with a heterologous polynucleotide include, e.g., Streptococcus (e.g., Streptococcus mitis), Haemophilus, Prevotella, Rothia mucilaginosa, and/or Corynebacterium matruchotii.

Main bacterial inhabitants of the stomach and which can be substantially isolated for transformation with a heterologous polynucleotide include, e.g., include: Streptococcus, Staphylococcus, Lactobacillus, Helicobacter and/or Peptostreptococcus.

Bacterial species commonly found in the human vagina and which can be substantially isolated for transformation with a heterologous polynucleotide include, e.g., Lactobacillus (e.g., including L. crispatus, L. iners, L. jensenii, or L. gasseri), Gardnerella and/or Prevotella.

Enrichment of the infant gut microbiome for symbionts such as Bacteroides, Parabacteroides, Clostridium, Lactobacillus, Bifidobacterium, and/or Faecalibacterium prausnitzii provides several determinants of a healthy microbiome. Such bacterial species can be transformed to express a heterologous polynucleotide.

Candidate colonies are restreaked on at least a second solid agar substrate to substantially isolate them from contaminating strains. Isolates of these purified strains can be stored as cryogenic stocks. The purified strains are subject to testing to confirm their genus/species identity, the absence of pathogenic toxins and susceptibility to clinically-used antibiotics. For example, PCR and Sanger sequencing, e.g., of all or part of a ribosomal 16S DNA sequence, can be performed to confirm genus/species identity.

The methods select against or select to eliminate commensal or native bacteria colonies that express pathogenic toxins. In some embodiments, bacterial cells are confirmed not to express any known pathogenic toxins or selected pathogenic toxins. In some embodiments bacterial cells are confirmed to not express one or more pathogenic toxins selected from the group consisting of AB toxin, Alpha toxin, Anthrax toxin, Botulinum toxin, Cereulide, Cholesterol-dependent cytolysin, Clostridial Cytotoxin family, Clostridium botulinum C3 toxin, Clostridium difficile toxin A, Clostridium difficile toxin B, Clostridium enterotoxin, Clostridium perfringens alpha toxin, Clostridium perfringens beta toxin, Cry1Ac, Cry6Aa, Cry34Ab1, Delta endotoxin, Diphtheria toxin, Enterotoxins, Enterotoxin type B, Erythrogenic toxin, Exfoliatin, Fragilysin, Haemolysin E, Heat-labile enterotoxin, Heat-stable enterotoxin, Hemolysin, HrpZ Family, Leukocidin, Listeriolysin O, Panton-Valentine leucocidin, intact Pathogenicity island, Phenol-soluble modulin, Pneumolysin, Pore-forming toxin, Pseudomonas exotoxin, Pyocyanin, anti-eukaryotic Rhs toxins, RTX toxin, Shiga toxins, Shiga-like toxin, Staphylococcus aureus alpha toxin, Staphylococcus aureus beta toxin, Staphylococcus aureus delta toxin, Streptolysin, Tetanolysin, Tetanospasmin, Toxic shock syndrome toxin, Tracheal cytotoxin, and/or Verocytotoxin.

The methods further select for commensal or native bacteria colonies that demonstrate sensitivity or susceptibility (e.g., lack of resistance) to clinically-used antibiotics. In some embodiments, bacterial cells are confirmed to be sensitive or susceptible (e.g., lack resistance) to one or more antibiotic agents selected from the group consisting of antibiotic macrolides (e.g., azithromycin, clarithromycin, erythromycin, fidaxomicin, telithromycin, carbomycin A, josamycin, kitasamycin, midecamycin/midecamycin acetate, oleandomycin, solithromycin, spiramycin, troleandomycin, tylosin/tylocine, or roxithromycin), rifamycins (e.g., rifampicin (or rifampin), rifabutin, rifapentine, rifalazil, or rifaximin), polymyxins (e.g., polymyxin B, or polymyxin E (colistin)), quinolone antibiotics (e.g., nalidixic acid, ofloxacin, levofloxacin, ciprofloxacin, norfloxacin, enoxacin, lomefloxacin, grepafloxacin, trovafloxacin, sparfloxacin, temafloxacin, moxifloxacin, gatifloxacin, or gemifloxacin), beta-lactams (e.g., penicillin, cloxacillin, dicloxacillin, flucloxacillin, methicillin, nafcillin, oxacillin, temocillin, amoxicillin, ampicillin, mecillinam, carbenicillin, ticarcillin, azlocillin, mezlocillin, or piperacillin), aminoglycosides (e.g., amikacin, gentamicin, neomycin, streptomycin, or tobramycin), cephalosporins (e.g., cefadroxil, cefazolin, cephalexin, cefaclor, cefoxitin, cefprozil, cefuroxime, loracarbef, cefixime, cefdinir, cefditoren, cefoperazone, cefotaxime, cefpodoxime, cefepime, or ceftobiprole), monobactams (e.g., aztreonam, tigemonam, nocardicin A, or tabtoxinine β-lactam), carbapenems (e.g., biapenem, doripenem, ertapenem, faropenem, imipenem, meropenem, panipenem, razupenem, tebipenem, or thienamycin), and/or tetracyclines (e.g., tetracycline, chlortetracycline, oxytetracycline, demeclocycline, lymecycline, meclocycline, methacycline, minocycline, rolitetracycline, or tigecycline). Generally, transformation of the purified native bacterial colony with a heterologous polynucleotide confers antibiotic resistance to one or more antibiotic agents used for selection of transformed bacterial cells, e.g., resistance to kanamycin, chloramphenicol, carbenicillin, hygromycin and/or trimethoprim.

Isolated colonies confirmed to not express known pathological toxins and to be susceptible to clinically-relevant antibiotic agents are transformed with one or more polynucleotides encoding one or more proteins that are heterologous to the bacteria and/or the intended host. In some embodiments, the heterologous protein is used for detection, e.g. a fluorescent protein. In some embodiments, the heterologous protein is a therapeutic polypeptide, as described in further detail below.

An isolated and substantially homogenous colony of native/commensal bacteria can be transformed using techniques known in the art. Such techniques are described, e.g., in Green and Sambrook, Molecular Cloning: A Laboratory Manual, Cold Spring Harbor Laboratory Press; 4th edition (2012). Clinical microbiology differentiation manuals to guide selection and identification of bacterial species of interest include, e.g., Medical Microbiology, 8th Edition by Murray, Rosenthal, and Pfaller, Elsevier, 2015; and Medical Microbiology: A Guide to Microbial Infections: Pathogenesis, Immunity, Laboratory Investigation and Control, 19th Edition, by Barer, Irving, Swann and Perera, Elsevier, 2018.

The isolated population of native/commensal bacteria are transformed, for example genetically modified, to express one or more heterologous polypeptides of interest, e.g., one or more therapeutic polypeptides listed in Table 1 and/or a detectable protein such as a fluorescent protein.

The polynucleotides encoding the heterologous polypeptides may be introduced in a vector, preferably expression vectors. “Vector” refers to a nucleic acid molecule capable of transporting another nucleic acid to which it has been linked. Expression vectors include one or more regulatory sequences and direct the expression of genes to which they are operably linked. By “operably linked” is intended that the nucleotide sequence of interest is linked to the regulatory sequence(s) such that expression of the nucleotide sequence is allowed (e.g., in an in vitro transcription/translation system or in a host cell when the vector is introduced into the host cell). The term “regulatory sequence” is intended to include controllable transcriptional promoters, operators, enhancers, transcriptional terminators, and other expression control elements such as translational control sequences (e.g., Shine-Dalgarno consensus sequence, initiation and termination codons). These regulatory sequences will differ, for example, depending on the host cell being used.

The polynucleotides encoding the heterologous polypeptides may be codon biased for improved expression in the native/commensal bacterial host cell. Preferred codon usage for the genera and species of the commensal or native bacterial host cells isolated and transformed, and described herein are known, and described in available codon usage databases, e.g., at kazusa.or.jp/codon/.

The vectors can be autonomously replicated in a host cell (episomal vectors), or may be integrated into the genome of a host cell, and replicated along with the host genome (non-episomal mammalian vectors). Integrating vectors typically contain at least one sequence homologous to the bacterial chromosome that allows for recombination to occur between homologous DNA in the vector and the bacterial chromosome. Integrating vectors may also comprise bacteriophage or transposon sequences. Episomal vectors, or plasmids are circular double-stranded DNA loops into which additional DNA segments can be ligated. Plasmids capable of stable maintenance in a host are generally the preferred form of expression vectors when using recombinant DNA techniques. Illustrative bacteriophage recombination systems of use are described, e.g., in Nafissi, et al., Appl Microbiol Biotechnol. 2014 April; 98(7):2841-51. A further bacteriophage delivery system of use is described, e.g., in U.S. Patent Publ. No. 2016/0367701. Commensal growth can have deleterious effects on plasmid maintenance. As needed, facilitating or promoting plasmid maintenance or retention in the transformed commensal/native bacterial host cells can be achieved using methods known in the art. Such plasmid retention or maintenance strategies include without limitation, e.g., partitioning systems (e.g., parABS; see, e.g., Yamaichi, et al., Proc Natl Acad Sci USA. (2000) 97(26):14656-61; Youngren, et al., J Bacteriol. 2000 July; 182(14):3924-8; Dubarry, et al., J Bacteriol. 2006 February; 188(4):1489-96; and Hanai, et al., J Biol. Chem. (1996) 271:17469-17475) or toxin-antitoxin modules or systems (e.g., ccdAB, hok-sok; see, e.g., Fernandez-Garcia, et al., Toxins (Basel). (2016) Jul. 20; 8(7). pii: E227); Fang, et al., Appl. Environ. Microbiol. (2008) 74(10):3216-3228; Lobato-Marquez, et al., Front Mol Biosci. 2016 Oct. 17; 3:66; Zielenkiewicz, et al., J. Bacteriol. (2005) 187(17):6094-6105; Leplae, et al., Nucleic Acids Research, (2011) 39(13) 5513-5525 and Grady, et al., Molecular Microbiology (2003) 47(5):1419-1432).

Regulatory sequences include those that direct constitutive expression of a nucleotide sequence as well as those that direct inducible expression of the nucleotide sequence only under certain environmental conditions. A bacterial promoter is any DNA sequence capable of binding bacterial RNA polymerase and initiating the downstream (3′) transcription of a coding sequence (e.g., structural gene) into mRNA. A promoter will have a transcription initiation region, which is usually placed proximal to the 5′ end of the coding sequence. This transcription initiation region typically includes an RNA polymerase binding site and a transcription initiation site. A bacterial promoter may also have a second domain called an operator, which may overlap an adjacent RNA polymerase binding site at which RNA synthesis begins. The operator permits negative regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence. Illustrative, regulator/promoter systems of use for expressing a heterologous polynucleotide in a transformed native/commensal bacterial cell include without limitation, e.g., XylS/Pm (wild-type), XylS/Pm ML1-17 (a Pm variant), LacI/PT7lac, LacI/Ptrc and/or AraC/PBAD. See, Balzar, et al., Microbial Cell Factories 2013, 12:26.

An example of a gene activator protein is the catabolite activator protein (CAP), which helps initiate transcription of the lac operon in Escherichia coli (Raibaud et al. (1984) Annu. Rev. Genet. 18:173). Regulated expression may therefore be either positive or negative, thereby either enhancing or reducing transcription. Other examples of positive and negative regulatory elements are well known in the art. Various promoters that can be included in the protein expression system include, but are not limited to, a T7/LacO hybrid promoter, a trp promoter, a T7 promoter, a lac promoter, p6 promoter, and a bacteriophage lambda promoter. Any suitable promoter can be used to carry out the present invention, including the native promoter or a heterologous promoter. Heterologous promoters may be constitutively active or inducible. A non-limiting example of a heterologous promoter is given in U.S. Pat. No. 6,242,194 to Kullen and Klaenhammer.

“Constitutive promoter” refers to a promoter that is capable of facilitating continuous transcription of a coding sequence or gene under its control and/or to which it is operably linked. Illustrative constitutive promoters of use include without limitation, e.g., BBa_J23100, a constitutive Escherichia coli σ^{S} promoter (e.g., an osmY promoter (International Genetically Engineered Machine (iGEM) Registry of Standard Biological Parts Name BBa_J45992; BBa_J45993)), a constitutive Escherichia coli σ³² promoter (e.g., htpG heat shock promoter (BBa_J45504)), a constitutive Escherichia coli σ⁷⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g., promoter veg (BBa_K143013), promoter 43 (BBa_K143013), PliaG (BBa_K823000), PlepA (BBa_K823002), Pveg (BBa_K823003)), a constitutive Bacillus subtilis σ^{B} promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), and/or a bacteriophage SP6 promoter (e.g., SP6 promoter (BBa_J64998)).

Examples of inducible promoters of use include, but are not limited to, an FNR promoter, a ParaC promoter, a ParaBAD promoter, a propionate promoter, and/or a PTetR promoter.

In order to maintain the ability for long-term or permanent colonization of a mammalian subjection (e.g., the ability for successful reintroduction into a mammalian microbiome), populations of native/commensal bacteria transformed to express one or more heterologous polypeptides are not adapted for laboratory or in vitro culture environments. The ENB are cultured in vitro in a laboratory environment for as few divisions as possible, In some embodiments, the ENB are cultured in vitro in a laboratory environment outside of the donating subject for 30 or fewer days, e.g., 25, 20, 15, 10 or fewer days before administration to the receiving subject. In some embodiments, the ENB have an overall in vitro growth time of about 14 or fewer days, e.g., 13, 12, 11, 10, 9, 8, 7 or fewer days between collection from the donating subject and administration to the receiving subject. Such calculations of in vitro growth time or culture time generally do not include time the bacterial cells are stored (e.g., cryopreserved or lyophilized), and include time for transformation or introduction of the one or more heterologous polynucleotides.

In some embodiments, the native/commensal host cells: (i) are capable of metabolizing one or more carbohydrates selected from the group consisting of sucrose, xylose, d-maltose, N-acetyl-d-glucosamine, d-galactose, and d-ribose; (ii) utilize both glycolytic and gluconeogenic substrates; (iii) are non-motile (e.g, have non-functioning flagella, e.g., due to a mutation in the flhDC operon); (iv) are capable of producing ribose-5-phosphate; (v) are able to grow in defined medium lacking vitamin B12 (cyanocobalamin) (e.g., are demonstrated vitamin B12 prototrophs); (vi) express an UDP-glucose-4-epimerase and/or a glycosyltransferase; (vii) comprise multiple copies of a gene encoding β subunit of tryptophan synthase gene; (viii) comprise multiple copies of a gene encoding propionate CoA-transferase; (ix) express capsular polysaccharide (CPS) 4 (CPS4); (x) express an rnf-like oxidoreductase complex; (xi) catabolize tryptophan to yield indole and other indole metabolites, e.g., indole-3-propionate and indole-3-aldehyde; and/or do not produce any agent that induces double-stranded DNA breaks, e.g., do not have a genomic island that encodes giant modular nonribosomal peptide and polyketide synthases, do not express hybrid peptide-polyketide genotoxins, and/or do not have an active clbA gene. Genotypes and phenotypes contributing to the ability of commensal bacteria to stably colonize on or in a mammal are described, e.g., in Lozupone, et al., Genome Res (2012) 22:1974-1984; Leatham, et al., Infect. Immun. (2005) 73(12):8039-8049; Miranda, et al., Infect. Immun. (2004) 72(3):1666-1676; Leatham, et al., Infect. Immun. (2009) 77(7):2876-2886 and Goodman, et al., Cell Host Microbe. 2009 Sep. 17; 6(3): 279-289. Others have found that laboratory adapted E. coli strains, e.g., Nissle 1917, induce DNA double-strand breaks. See, e.g., Nougayrède, et al., Science (2006) 313(5788):848-851; and Olier, et al., Gut Microbes. (2012) 3(6): 501-509. Populations of native/commensal bacteria transformed to express one or more heterologous polypeptides can be cryopreserved or lyophilized for long-term storage.

2. Conditions Subject to Treatment, Amelioration and/or Prevention

Depending on the heterologous polynucleotide expressed by the ENBs and the route of administration, the ENBs described herein find use in treating or preventing numerous disease conditions, as summarized in Table 1.

For example, in some embodiments, a population of native/commensal bacteria cells transformed to express a bile salt hydrolase (e.g., from Lactobacillus or Bifidobacterium) can be administered to the gastrointestinal tract, e.g., orally and/or rectally, to mitigate, ameliorate, reduce, inhibit, reverse and/or prevent one or more symptoms caused by or associated with obesity/type 2 diabetes, chronic kidney disease, cognitive decline/deficiency (e.g., due to traumatic brain injury, dementia, stroke, hepatic encephalopathy, infant anoxic brain injury), hypercholesterolemia, male infertility, female infertility, C. difficile infection. In some embodiments, the heterologous polynucleotide encodes a bile salt hydrolase having at least or at least about 80%, 85%, 90%, 95%, 97%, 99% or 100% sequence identity to GenBank: ACL98194.1 (Lactobacillus salivarius BSH); to NCBI Reference Sequence: YP_193782.1 (Lactobacillus acidophilus bshA); to NCBI Reference Sequence: YP_193954.1 (Lactobacillus acidophilus bshB); or to Gene ID: 31838777 (Bifidobacterium thermophilum RBL67 conjugated bile salt hydrolase D805_RS01800).

In some embodiments, a population of native/commensal bacteria cells transformed to express a mammalian (e.g., human) sulfotransferase family 2A member 1 (SULT2A1) can be administered to the gastrointestinal tract, e.g., orally and/or rectally, to mitigate, ameliorate, reduce, inhibit, reverse and/or prevent one or more symptoms caused by or associated with Obesity/Type 2 Diabetes, Hypercholesterolemia, Non-alcoholic steatohepatitis (NAFLD) and Dementia/Cognitive decline.

In some embodiments, a population of native/commensal bacteria cells transformed to express a mammalian (e.g., human) NAPE-hydrolyzing phospholipase D (NAPEPLD), FGF1, FGF15, FGF19 and/or glucacon (GLP-1) can be administered to the gastrointestinal tract, e.g., orally and/or rectally, to mitigate, ameliorate, reduce, inhibit, reverse and/or prevent one or more symptoms caused by or associated with Obesity/Type 2 Diabetes.

In some embodiments, one or more populations of native/commensal bacteria cells transformed to express a mammalian (e.g., human) IL-10, TGFβ, IL-27 dimer and/or anti-TNFα antibody can be administered to the gastrointestinal tract, e.g., orally and/or rectally, to mitigate, ameliorate, reduce, inhibit, reverse and/or prevent one or more symptoms caused by or associated with an autoimmune disease, e.g., ulcerative colitis, Crohn's disease, Type 1 or autoimmune diabetes.

In some embodiments, a population of native/commensal bacteria cells transformed to express a mammalian (e.g., human) trefoil factor (e.g., TFF1, TFF2 and/or TFF3) or peptidase inhibitor 3 (PI3) or Elafin (Serpina1c) can be administered to the gastrointestinal tract, e.g., orally and/or rectally, to mitigate, ameliorate, reduce, inhibit, reverse and/or prevent one or more symptoms caused by or associated with an inflammatory disease, e.g., oral mucositis, ulcerative colitis, Crohn's disease.

In some embodiments, a population of native/commensal bacteria cells transformed to express Vibrio vulnificus flagellin B can be administered to the gastrointestinal tract, e.g., orally and/or rectally, to mitigate, ameliorate, reduce, inhibit, reverse and/or prevent one or more symptoms caused by or associated with cancer, e.g., a cancer of the gastrointestinal tract, e.g., oral cancer, esophageal cancer, stomach cancer, colon cancer, or rectal cancer.

In some embodiments, a population of native/commensal bacteria cells transformed to express Nostoc elipsosporum cyanovirin-N can be administered to the gastrointestinal tract, e.g., orally and/or rectally, and/or to the genitourinary tract, to mitigate, ameliorate, reduce, inhibit, reverse and/or prevent one or more symptoms caused by or associated with HIV.

In some embodiments, a population of native/commensal bacteria cells transformed to express Actinobacillus actinomycetemcomitans dispersin B (DspB) can be administered to the gastrointestinal tract, e.g., orally and/or rectally, to mitigate, ameliorate, reduce, inhibit, reverse and/or prevent one or more symptoms caused by or associated with Pseudomonas aeruginosa infection.

In some embodiments, a population of native/commensal bacteria cells transformed to express Microcin J25 (MccJ25) can be administered to the gastrointestinal tract, e.g., orally and/or rectally, to mitigate, ameliorate, reduce, inhibit, reverse and/or prevent one or more symptoms caused by or associated with Salmonella enterica infection.

In some embodiments, a population of native/commensal bacteria cells transformed to express cholylglycine hydrolase and/or cholic acid 7alpha-dehydroxylase can be administered to the gastrointestinal tract, e.g., orally and/or rectally, to mitigate, ameliorate, reduce, inhibit, reverse and/or prevent one or more symptoms caused by or associated with Clostridium difficile infection.

In some embodiments, a population of native/commensal bacteria cells transformed to express Akkermansia muciniphila Amuc_1100* can be administered to the gastrointestinal tract, e.g., orally and/or rectally, to mitigate, ameliorate, reduce, inhibit, reverse and/or prevent one or more symptoms caused by or associated with Non-alcoholic steatohepatitis (NAFLD) and/or Aging/Senescence.

In some embodiments, a population of native/commensal bacteria cells transformed to express bile acid-CoA:amino acid N-acyltransferase (BAAT) can be administered to the gastrointestinal tract, e.g., orally and/or rectally, to mitigate, ameliorate, reduce, inhibit, reverse and/or prevent one or more symptoms caused by or associated with malnutrition.

In some embodiments, a population of native/commensal bacteria cells transformed to express a mammalian (e.g., human) lactase can be administered to the gastrointestinal tract, e.g., orally and/or rectally, to mitigate, ameliorate, reduce, inhibit, reverse and/or prevent one or more symptoms caused by or associated with lactose intolerance.

In some embodiments, a population of native/commensal bacteria cells transformed to express a mammalian (e.g., human) phenylalanine hydroxylase can be administered to the gastrointestinal tract, e.g., orally and/or rectally, to mitigate, ameliorate, reduce, inhibit, reverse and/or prevent one or more symptoms caused by or associated with phenylketouria.

In some embodiments, a population of native/commensal bacteria cells transformed to express a mammalian (e.g., a human) alcohol dehydrogenase can be administered to the gastrointestinal tract, e.g., orally and/or rectally, to mitigate, ameliorate, reduce, inhibit, reverse and/or prevent one or more symptoms caused by or associated with alcohol intolerance/toxicity.

In some embodiments, a population of native/commensal bacteria cells transformed to express an Aspergillus niger-derived prolyl endoprotease (AN-PEP) can be administered to the gastrointestinal tract, e.g., orally and/or rectally, to mitigate, ameliorate, reduce, inhibit, reverse and/or prevent one or more symptoms caused by or associated with celiac disease.

In some embodiments, a population of native/commensal bacteria cells transformed to express 7-alpha-hydroxysteroid dehydrogenase (hdhA) and/or 7 beta-hydroxysteroid dehydrogenase (7β-HSDH; EC 1.1.1.201) can be administered to the gastrointestinal tract, e.g., orally and/or rectally, to mitigate, ameliorate, reduce, inhibit, reverse and/or prevent one or more symptoms caused by or associated with traumatic brain injury, dementia/cognitive decline, hepatic encephalopathy, infant anoxic brain injury.

The mammal subject to such therapies may be exhibiting symptoms or be asymptomatic. The mammal may have a familial history or a determined genetic risk for the disease condition. The mammal may be an adult, a juvenile, a child or an infant.

When delivered to the gastrointestinal tract, the engineered native bacteria (ENB) do not significantly alter the gastrointestinal microbiome, e.g., the terminal ileum microbiome, regardless of presence or absence of the therapeutic polypeptide. This can be confirmed by analysis, e.g., sequencing of the ribosomal 16S DNA of the microbiome, e.g., before and after administration of the ENBs. Accordingly, in some embodiments, a subject is selected or identified to be one within a class of subjects in need of such therapies and the selection or identification can be made by clinical or diagnostic evaluation.

TABLE 1
HETEROLOGOUS POLYPEPTIDES THAT CAN BE EXPRESSED IN ENGINEERED NATIVE BACTERIA
AND CONDITIONS SUBJECT TO PREVENTION AND TREATMENT

| Disease/Condition | | | | | | |
|---|---|---|---|---|---|---|
| Subject to Treatment | Gene Knocked In | Function | Gene Name | Gene ID | | Reference |
| Obesity/Type 2 Diabetes | Bile Salt Hydrolase (e.g., | Increase: | bshA | 3251811 | | Joyce, et al. Proc Natl |
| Chronic Kidney disease | from Lactobacillus) | FXR activity/expression | bshB | 3252955 | | Acad Sci USA. 2014 |
| Dementia/Cognitive decline | | FGF15/FGF19 production | | | | May 20; 111(20): 7421-6; |
| Hepatic Encephalopathy | | Bile acid (BA) excretion | | | | Yoon, et al., J Microbiol. |
| Infant anoxic brain injury | | Physical activity | | | | 2017 November; |
| Hypercholesterolemia | | Decrease: | | | | 55(11): 892-899 |
| Increase male and female | | lithocholic acid (LCA) | | | | |
| fertility | | deoxycholic acid (DCA) | | | | |
| C difficile infection | | neuroinflammation | | | | |
| Obesity/Type 2 Diabetes | sulfotransferase family | Increase: | SULT2A1 | 6822 | (human) | Wahlström, et al., Cell |
| Hypercholesterolemia | 2A member 1 | Bile acid (BA) excretion | | 20859 | (mouse) | Metab. 2016 Jul. |
| Non-alcoholic steatohepatitis | | Decrease: | | | | 12; 24(1): 41-50; Quinn, |
| (NAFLD) | | lithocholic acid | | | | et al., Dig Liver Dis. |
| Dementia/Cognition | | deoxycholic acid | | | | 2014 June; 46(6): 527-34 |
| Obesity/Type 2 Diabetes | NAPE-hydrolyzing | Increased Satiety | NAPEPLD | 222236 | (human) | Chen, et al., J Clin |
| | phospholipase D | | | 242864 | (mouse) | Invest. 2014 August; |
| | | | | | | 124(8): 3391-406 |
| Type 1 and Type 2 | whole proinsulin | Mitigate autoimmune | INS | 3630 | (human) | Takiishi, et al., J Clin |
| Diabetes/Non-alcoholic | autoantigen and IL-10 | response | | 16334 | (mouse) | Invest. 2012 |
| Steatohepatitis | | | IL10 | 3586 | (human) | May; 122(5): 1717-25 |
| | | | | 16153 | (mouse) | |
| Cancer Immunotherapy | Vibrio vulnificus | FlaB-mediated tumor | WCECT4999_ | 33953481 | | Zheng, et al., Sci Transl |
| | flagellin B | suppression | RS04515 | | | Med. 2017 Feb. 8; 9(376) |
| Ulcerative Colitis/ | Epstein-Barr virus | Increase IL-27 | IL27 | 246778 | (human) | Hanson, et al., |
| Crohn's Disease | induced 3 (EBI3) + p28, | production | | 246779 | (mouse) | Gastroenterology. 2014 |
| | e.g., as fusion gene | | EBI3 | 10148 | (human) | January; 146(1): 210-221 |
| | (IL-27 is a heterodimer) | | | 50498 | (mouse) | |
| Ulcerative Colitis/ | IL-10 gene | Increase IL-10 | IL10 | 3586 | (human) | Steidler et al., Science. |
| Crohn's Disease | | production | | 16153 | (mouse) | 2000 Aug. |
| Type 1 diabetes | | | | | | 25; 289(5483): 1352-5 |
| Ulcerative Colitis/ | TGFβ | Increase TGF-β | TFB1 | 7040 | (human) | |
| Crohn's Disease | | production | | 21803 | (mouse) | |
| Type 1 diabetes | | | | | | |
| Ulcerative Colitis/ | peptidase inhibitor 3 | Protease inhibitor with | PI3 | 5266 | (human) | Motta, et al., Sci Transl |
| Crohn's Disease | (PI3) or Elafin | anti-inflammatory | | 20702 | (mouse) | Med. 2012 Oct. |
| | | effects | Serpina1c | | | 31; 4(158): 158ra144 |
| Ulcerative Colitis/ | Trefoil factors (TFF) | Improve intestinal | TFF1 | 7031 | (human) | Vandenbroucke, et al., |
| Crohn's Disease | TFF1, TFF2, TFF3 | epithelial barrier | | 21784 | (mouse) | Gastroenterology. 2004 |
| Oral mucositis | | | TFF2 | 7032 | (human) | August; 127(2): 502-13 |
| | | | | 21785 | (mouse) | |
| | | | TFF3 | 7033 | (human) | |
| | | | | 21786 | (mouse) | |
| Ulcerative Colitis/ | monovalent and bivalent | Anti-TNFα; anti- | | | | Vandenbroucke, et al., |
| Crohn's Disease | murine (m)TNF- | inflammatory | | | | Mucosal lmmunol. 2010 |
| | neutralizing Nanobodies | | | | | January; 3(1): 49-56 |
| HIV | Nostoc elipsosporum | HIV-1 entry inhibitor | | | | Lagenaur, et al., |
| | cyanovirin-N | | | | | Mucosal Immunol. 2011 |
| | | | | | | November; 4(6): 648-57 |
| Pseudomonas aeruginosa | Actinobacillus | synthetic genetic system | DspB | | | Hwang, et al., Nat |
| infection | actinomycetemcomitans | that allows bacteria to | | | | Commun. 2017 Apr. |
| | dispersin B (DspB) | sense and kill | | | | 11; 8: 15028; Itoh, Y. et |
| | | | | | | al. J. Bacteriol. 2005 |
| | | | | | | 187: 382-387 |
| Salmonella enterica | Microcin J25 (MccJ25), | | MccJ25 | | | Forkus, et al., Sci Rep. |
| infections | a 21-residue | | | | | 2017 Jan. 17; 7: 40695; |
| | peptide natively | | | | | Blond, A. et al. Eur. J. |
| | secreted from | | | | | Biochem. 1999 |
| | the human E. coli | | | | | 259: 747-755; U.S. |
| | isolate AY2514 | | | | | Pat. No. 8,461,314 |
| Obesity/Type 2 Diabetes | butyrate pathway genes, | Altered glucose | | | | Baek, et al., Biotechnol |
| | e.g., butyrylCoA: | homeostasis | | | | Bioeng. 2013 |
| | acetate CoA transferase; | | | | | October; 110(10): 2790-4 |
| | 3-hydroxy-butyryl CoA | | | | | |
| | dehydratase (K01715; | | | | | |
| | EC: 4.2.1.55), butyryl- | | | | | |
| | CoA dehydrogenase | | | | | |
| | [EC: 1.3.99.2]; | | | | | |
| Obesity/Type 2 Diabetes | Fibroblast growth | Increased FGF1 | FGF1 | 2246 | (human) | Suh, et al., Nature. |
| | factor 1 | production | | 14164 | (mouse) | 2014 Sep. |
| | | | | | | 18; 513(7518): 436-9 |
| Obesity/Type 2 Diabetes | Fibroblast growth | Increased FGF15/FGF19 | FGF19 | 9965 | (human) | Benoit et al., Nat Med. |
| | factor 19 | production | FGF15 | 14170 | (mouse) | 2017 August; 23(8): 990- |
| | | | | | | 996 |
| Obesity/Type 2 Diabetes | Glucacon (GLP-1) | Increase incretin release; | GCG | 2641 | (human) | Duan, et al. Diabetes. |
| Short Bowel Syndrome | | Convert gut epithelium | | 14526 | (mouse) | 2015 May; 64(5): 1794- |
| | | into insulin secreting cells | | | | 803 |
| Malnutrition/weight gain | BA inducible gene B | Conjugation of bile acids | BAAT | 570 | (human) | |
| (e.g. agricultural) | (baiB) + bile | (BAs) | | 12012 | (mouse) | |
| | acid-CoA:amino acid N- | | | | | |
| | acyltransferase (BAAT) | | | | | |
| Non-alcoholic steatohepatitis | Akkermansia muciniphila | Decrease gut permeability | AMUC_RS0 | 34174504 | | Plovier, et al, Nat Med. |
| (NAFLD) | Amuc_1100* | | 5900 | | | 2017 January; 23(1): |
| Aging/Senescence | | | | | | 107-113 Thevaranjan, |
| | | | | | | et al., Cell Host Microbe. |
| | | | | | | 2017 Apr. 12; |
| | | | | | | 21(4): 455-466.e4 |
| EtOH toxicity/“Asian flush” | EtOH dehydrogenase | Increased alcohol | ADH1A, | | | |
| | | metabolism; converts | ADH1B, | | | |
| | | EtOH early | ADH1C, | | | |
| | | | ADH2, | | | |
| | | | ADH3, | | | |
| | | | ADH4, | | | |
| | | | ADH5, | | | |
| | | | ADH6, | | | |
| | | | ADH7 | | | |
| Clostridium difficile | cholylglycine hydrolase | Increase lithocholic acid | | | | Thanissery et al., |
| Infection | cholic acid 7alpha- | (LCA) and deoxycholic | | | | Anaerobe. 2017 |
| | dehydroxylase | acid (DCA) in colon | | | | June; 45: 86-100 |
| Lactose intolerance | Lactase (beta- | Increased lactase | LCT | 3938 | (human) | |
| | galactosidase) | | | 226413 | (mouse) | |
| Phenylketouria | phenylalanine | Degrade phenylalanine | PAH | 5053 | (human) | Martinez, et al., |
| | hydroxylase | | | 18478 | (mouse) | Biochem J. 1995 Mar. |
| | | | | | | 1; 306 (Pt 2): 589-97 |
| Traumatic brain injury | 7-alpha- | Decrease | hdhA | 946151 | (E. coli) | Nunes, et al., Mol |
| Dementia/Cognitive decline | hydroxysteroid | neuroinflammation | 7β-HSDH; | | | Neurobiol. 2012 |
| Hepatic Encephalopathy | dehydrogenase + | Increase | EC 1.1.1.201 | | | June; 45(3): 440-54; |
| Infant anoxic brain injury | 7 beta- | ursodeoxycholic | | | | Yanguas-Casás, et al., |
| | hydroxysteroid | acid (UDCA) | | | | J Cell Physiol. 2017 |
| | dehydrogenase | | | | | August; 232(8): 2231-2245; |
| | | | | | | Zheng, et al., J Agric |
| | | | | | | Food Chem. 2017 Feb. |
| | | | | | | 15; 65(6): 1178-1185; |
| | | | | | | Shi, et al., J Ind |
| | | | | | | Microbiol Biotechnol. |
| | | | | | | 2017 July; 44(7): 1073- |
| | | | | | | 1082 |
| Celiac Disease | Aspergillus niger- | Degradation of gluten | | | | Kaukinen and Lindfors, |
| | derived prolyl | | | | | Dig Dis. |
| | endoprotease | | | | | 2015; 33(2): 277-81; |
| | (AN-PEP) | | | | | König, et al., Sci Rep. |
| | | | | | | 2017 Oct. |
| | | | | | | 12; 7(1): 13100. |

3. Formulation and Administration

The native/commensal bacteria transformed to express a heterologous polynucleotide (ENBs) can be formulated into bacterial compositions for administration to humans and other mammalian subjects in need thereof. Generally, the bacterial compositions are combined with additional active and/or inactive materials in order to produce a final product, which may be in single dosage unit or in a multi-dose format. In some embodiments, the bacterial compositions are comprised of one or more ENB populations, as described herein. In some embodiments, the bacterial compositions are comprised of one or more ENB populations and one or more prebiotics.

The composition(s) may include different types of carriers depending on whether it is to be administered in solid or liquid. The ENB compositions can be administered orally, intravaginally, intrarectally, topically (e.g., including into the eye or conjunctiva), intratumorally, via vesicle instillation (e.g., into the bladder), intralesionally, intranasally, topically, or buccally. In various embodiments, the compositions can be delivered, e.g., via food, drink, capsule, gavage, enema, suppository, infusion, continuous infusion, localized perfusion bathing target cells directly, via a catheter, via a lavage, in lipid compositions (e.g., liposomes), as an aerosol, or by other method or any combination of the foregoing as would be known to one of ordinary skill in the art (see, for example, Lloyd V. Allen, Jr., Remington: The Science and Practice of Pharmacy, 22nd Edition, 2012, Pharmaceutical Press, expressly incorporated herein by reference in its entirety).

In some embodiments, the compositions comprise at least one prebiotic carbohydrate. A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate can be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Illustrative disaccharides include sucrose, maltose, cellobiose, and lactose. Typically, an oligosaccharide includes between three and six monosaccharide units (e.g., raffinose, or stachyose), and polysaccharides include six or more monosaccharide units. Exemplary polysaccharides include starch, glycogen, and/or cellulose. Carbohydrates can contain modified saccharide units, such as 2′-deoxyribose wherein a hydroxyl group is removed, 2′-fluororibose wherein a hydroxyl group is replace with a fluorine, or N-acetylglucosamine, a nitrogen-containing form of glucose (e.g., 2′-fluororibose, deoxyribose, and/or hexose). Carbohydrates can exist in many different forms, for example, conformers, cyclic forms, acyclic forms, stereoisomers, tautomers, anomers, and/or isomers.

In some embodiments, the compositions comprise at least one lipid. As used herein, a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans). In some embodiments, the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and/or tetracosanoic acid (24:0). In other embodiments, the composition comprises at least one modified lipid, for example, a lipid that has been modified by cooking.

In some embodiments, the composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and/or selenium. Suitable forms of any of the foregoing minerals include soluble mineral salts, slightly soluble mineral salts, insoluble mineral salts, chelated minerals, mineral complexes, non-reactive minerals such as carbonyl minerals, and/or reduced minerals, and combinations thereof.

In certain embodiments, the composition comprises at least one supplemental vitamin and/or an antioxidant. The at least one vitamin can be fat-soluble or water soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and/or biotin. Suitable forms of any of the foregoing are salts of the vitamin, derivatives of the vitamin, compounds having the same or similar activity of the vitamin, and metabolites of the vitamin.

In other embodiments, the composition comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and/or a coloring agent.

In another embodiment, the excipient is a buffering agent. Non-limiting examples of suitable buffering agents include sodium citrate, magnesium carbonate, magnesium bicarbonate, calcium carbonate, and/or calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and/or phenol.

In cases where a formulation contains anerobic bacterial strains, the pharmaceutical formulation and excipients can be selected to prevent exposure of the bacterial strains to oxygen.

In other embodiments, the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, or oligosaccharides, and combinations thereof.

In another embodiment, the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and/or light mineral oil.

In other embodiments, the composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and/or microcrystalline cellulose as high HLB emulsifier surfactants.

In some embodiments, the composition comprises a disintegrant as an excipient. In other embodiments, the disintegrant is a non-effervescent disintegrant. Non-limiting examples of suitable non-effervescent disintegrants include starches such as corn starch, potato starch, pregelatinized and/or modified starches thereof, sweeteners, clays, such as bentonite, micro-crystalline cellulose, alginates, sodium starch glycolate, or gums such as agar, guar, locust bean, karaya, pecitin, and/or tragacanth. In another embodiment, the disintegrant is an effervescent disintegrant. Non-limiting examples of suitable effervescent disintegrants include sodium bicarbonate in combination with citric acid, and/or sodium bicarbonate in combination with tartaric acid.

In another embodiment, the excipient comprises a flavoring agent. Flavoring agents can be chosen from synthetic flavor oils and/or flavoring aromatics; natural oils; extracts from plants, leaves, flowers, and/or fruits; and combinations thereof. In some embodiments the flavoring agent is selected from cinnamon oils; oil of wintergreen; peppermint oils; clover oil; hay oil; anise oil; eucalyptus; vanilla; citrus oil such as lemon oil, orange oil, grape and/or grapefruit oil; and/or fruit essences including apple, peach, pear, strawberry, raspberry, cherry, plum, pineapple, and/or apricot.

In other embodiments, the excipient comprises a sweetener. Non-limiting examples of suitable sweeteners include glucose (corn syrup), dextrose, invert sugar, fructose, and/or mixtures thereof (when not used as a carrier); saccharin and/or its various salts such as the sodium salt; dipeptide sweeteners such as aspartame; dihydrochalcone compounds, glycyrrhizin; Stevia Rebaudiana (Stevioside); chloro derivatives of sucrose such as sucralose; and/or sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and/or sodium and calcium salts thereof.

In some embodiments, the composition comprises a coloring agent. Non-limiting examples of suitable color agents include food, drug and cosmetic colors (FD&C), drug and cosmetic colors (D&C), and/or external drug and cosmetic colors (Ext. D&C). The coloring agents can be used as dyes or their corresponding lakes.

In various embodiments, the weight fraction of the excipient or combination of excipients in the formulation is usually about or at 99% or less (but not zero), such as about or at 95% or less (but not zero), about or at 90% or less (but not zero), about or at 85% or less (but not zero), about or at 80% or less (but not zero), about or at 75% or less (but not zero), about or at 70% or less (but not zero), about or at 65% or less (but not zero), about or at 60% or less (but not zero), about or at 55% or less (but not zero), about or at 50% or less (but not zero), about or at 45% or less (but not zero), about or at 40% or less (but not zero), about or at 35% or less (but not zero), about or at 30% or less (but not zero), about or at 25% or less (but not zero), about or at 20% or less (but not zero), about or at 15% or less (but not zero), about or at 10% or less (but not zero), about or at 5% or less (but not zero), about or at 2% or less (but not zero), or about or at 1% or less (but not zero) of the total weight of the composition.

Solid dosage forms for oral administration include capsules, tablets, caplets, pills, troches, lozenges, powders, and/or granules. A capsule typically comprises a core material comprising a bacterial composition and a shell wall that encapsulates the core material. In some embodiments, the core material comprises at least one of a solid, a liquid, and/or an emulsion. In other embodiments, the shell wall material comprises at least one of a soft gelatin, a hard gelatin, and/or a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and/or copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and/or copolymers such as polyvinyl pyrrolidone, polyvinyl acetate, polyvinylacetate phthalate, vinylacetate crotonic acid copolymer, and/or ethylene-vinyl acetate copolymers; and/or shellac (purified lac). In yet other embodiments, at least one polymer functions as taste-masking agents.

Tablets, pills, and the like can be compressed, multiply compressed, multiply layered, and/or coated. The coating can be single or multiple. In one embodiment, the coating material comprises at least one of a saccharide, a polysaccharide, and/or glycoproteins extracted from at least one of a plant, a fungus, and/or a microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments the coating material comprises a protein. In another embodiment, the coating material comprises at least one of a fat and an oil. In other embodiments, the at least one of a fat and an oil is high temperature melting. In yet another embodiment, the at least one of a fat and an oil is hydrogenated or partially hydrogenated. In one embodiment, the at least one of a fat and an oil is derived from a plant. In other embodiments, the at least one of a fat and an oil comprises at least one of glycerides, free fatty acids, and/or fatty acid esters. In some embodiments, the coating material comprises at least one edible wax. The edible wax can be derived from animals, insects, or plants. Non-limiting examples include beeswax, lanolin, bayberry wax, carnauba wax, and/or rice bran wax. Tablets and pills can additionally be prepared with enteric coatings.

Alternatively, powders or granules embodying the bacterial compositions disclosed herein can be incorporated into a food product. In some embodiments, the food product is a drink for oral administration. Non-limiting examples of a suitable drink include fruit juice, a fruit drink, an artificially flavored drink, an artificially sweetened drink, a carbonated beverage, a sports drink, a liquid diary product, a shake, an alcoholic beverage, a caffeinated beverage, or infant formula. Other suitable products for oral administration include aqueous and nonaqueous solutions, emulsions, suspensions and/or solutions and/or suspensions reconstituted from non-effervescent granules, containing at least one of suitable solvents, preservatives, emulsifying agents, suspending agents, diluents, sweeteners, coloring agents, and/or flavoring agents.

In some embodiments, the food product can be a solid foodstuff. Suitable examples of a solid foodstuff include without limitation a food bar, a snack bar, a cookie, a brownie, a muffin, a cracker, ice cream or an ice cream bar, yogurt or a frozen yogurt bar.

In other embodiments, the compositions disclosed herein are incorporated into a therapeutic food. In some embodiments, the therapeutic food is a ready-to-use food that optionally contains some or all essential macronutrients and micronutrients. In another embodiment, the compositions disclosed herein are incorporated into a supplementary food that is designed to be blended into an existing meal. In one embodiment, the supplemental food or neutraceutical contains some or all essential macronutrients and micronutrients. In another embodiment, the bacterial compositions disclosed herein are blended with or added to an existing food to fortify the food's protein nutrition. Examples include food staples (grain, salt, sugar, cooking oil, or margarine), beverages (coffee, tea, soda, waters, beer, liquor, or sports drinks), snacks, or sweets and other foods.

In one embodiment, the formulations are filled into gelatin capsules for oral administration. An example of an appropriate capsule is a 250 mg gelatin capsule containing from 10 (up to 100 mg) of lyophilized powder (e.g., from 10⁸ to 10¹¹ bacteria cells), 160 mg microcrystalline cellulose, 77.5 mg gelatin, and 2.5 mg magnesium stearate. In an alternative embodiment, from 10⁵ to 10¹² bacteria cells may be used, e.g., 10⁵ to 10⁷, 10⁶ to 10⁷, or 10⁸ to 10¹⁰ bacteria cells, with attendant adjustments of the excipients if necessary. In an alternative embodiment, an enteric-coated capsule or tablet or with a buffering or protective composition can be used.

The bacterial compositions, with or without one or more prebiotics, are generally formulated for oral or gastric administration, typically to a mammalian subject. In particular embodiments, the composition is formulated for oral administration as a solid, semi-solid, gel, or liquid form, such as in the form of a pill, tablet, capsule, or lozenge. In some embodiments, such formulations contain or are coated by an enteric coating to protect the bacteria through the stomach and small intestine, although spores are generally resistant to the stomach and small intestines. In other embodiments, the bacterial compositions, with or without one or more prebiotics, may be formulated with a germinant to enhance engraftment, or efficacy. In yet other embodiments, the bacterial compositions may be co-formulated or co-administered with prebiotic substances, to enhance engraftment or efficacy. In some embodiments, bacterial compositions may be co-formulated or co-administered with prebiotic substances, to enhance engraftment or efficacy.

The bacterial compositions, with or without one or more prebiotics, may be formulated to be effective in a given mammalian subject in a single administration or over multiple administrations. For example, a single administration is substantially effective to reduce or increase a monitored symptom or a biomarker of a targeted disease condition, e.g., increased insulin, increased metabolism, increased cognitive abilities, reduced inflammatory and/or autoimmune response, in a mammalian subject to whom the composition is administered. Substantially effective means that the monitored symptom or biomarker is reduced or increased in presence in the subject by at least 10%, 20%, 30%, 40%, 50%, 60%, 70%, 80%, 90%, 95%, 98%, 99% or greater than 99% following administration of the composition.

In some embodiments, the composition is formulated such that a single oral dose contains at least or at least about 1×10⁴ colony forming units of the bacterial entities and/or fungal entities, and a single oral dose will typically contain about or at 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or greater than 1×10¹⁵ CFUs of the bacterial entities. If known, for example the concentration of cells of a given strain, or the aggregate of all strains, is e.g., 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or greater than 1×10¹⁵ viable bacterial entities (e.g., CFUs) per gram of composition or per administered dose.

In some formulations, the composition contains at least or at least about 0.5%, 1%, 2%, 5%, 10%, 20%, 30%, 40%, 50%, 60%, 70%, 80%, 90% or greater than 90% bacterial cells on a mass basis. In some formulations, the administered dose does not exceed 200, 300, 400, 500, 600, 700, 800, 900 milligrams or 1, 1.1, 1.2, 1.3, 1.4, 1.5, 1.6, 1.7, 1.8, or 1.9 grams in mass.

4. Kits

Further provided are kits comprising one or more containers comprising one or more isolated populations of engineered native bacteria (ENB), or one or more compositions comprising one or more isolated populations of engineered native bacteria, as described herein. In various embodiments, the containers may comprise multiple unitary portions or doses of a composition comprising populations of ENBs that that been transformed to express the same one or more heterologous polynucleotides. In various embodiments, the containers may comprise multiple unitary portions or doses of compositions comprising populations of ENBs that that been transformed to express different one or more heterologous polynucleotides. In various embodiments, the containers contain edible compositions, e.g., food product, beverages, or capsules. In various embodiments, the containers contain unitary volumes of buffered solutions or suspensions comprising populations of ENBs, e.g., that that been transformed to express the same one or more heterologous polynucleotides. In various embodiments, the containers contain unitary doses of lyophilized ENBs, e.g., and buffered solution for reconstituting.

### EXAMPLES

The following examples are offered to illustrate, but not to limit the claimed invention.

### Example 1

### Illustrative Methods of Preparation of Engineered Commensal Bacteria

Sample Collection.

For human patients, mucosal biopsies of the duodenum, ileum, and colon were taken during endoscopy and suspended in 1.8 mL of 1×PBS in a 15 mL conical tube at room temperature. Within 8 hours, samples were transferred to a sterile 2 mL screw-top microcentrifuge tube, and a sterile 1.5 mm-diameter chrome-coated steel bead was added.

For mice, feces was collected by placing the mice in a sterile 1 L polypropylene cup until they defecated 2-5 whole pellets. Mice were returned to their home cages and the pellets were collected using sterile forceps and placed into sterile pre-weighed 2 mL screw-top microcentrifuge tubes. Tissue samples were collected after euthanasia by carbon dioxide and cervical dislocation and placed in sterile, pre-weighed 2 mL screw-top microcentrifuge tubes. For both tissue and feces, the following workup was the same. Within 2 hours, the tube was re-weighed and 1 mL of sterile deionized water was added to each tube followed by a sterile 1.5 mm-diameter chrome bead.

Sample Processing.

These samples were homogenized with a BioSpec Bead Beater Mini 24 for 1 minute at 3800 RPM, cooled on ice for approximately 1 minute, and homogenized for another 1 minute at 3800 RPM, and again cooled for approximate 1 minute on ice. Chrome-steel beads were removed by dragging a rare earth magnet up the side of the tube to remove the bead.

For human tissue samples, the resulting homogenates were placed in an Eppendorf 5430R centrifuge for 1 minute at 14,000 RPM to pellet cells and tissue. Of the resulting supernatant, 1.5 mL was transferred to a fresh, clean screw-top microcentrifuge tube and stored at room temperature. The pellet was resuspended in the remaining supernatant by vortexing the microcentrifuge tube for 30-60 seconds.

For mouse samples, the homogenized samples were subsequently used directly.

Strain Isolation.

For each of the resulting homogenized samples, 200 μL was dispensed onto disposable 10-cm Petri dishes containing 25 mL of hardened selective agar media and spread using 10-50 sterile 1 mm-diameter glass beads until the water was evaporated or absorbed by the media. Samples were subsequently diluted 10-fold before plating 100 μL of the diluted sample onto the same plates. Such dilution platings were performed up to 1000-fold dilution.

For the isolation of Escherichia coli strains, the selective agar media was either MacConkey Lactose Agar (“MacConkey”) or Eosin-Methylene Blue Lactose Agar (“EMB”). For the isolation of Lactobacillus strains, the selective agar media was De Man-Rogosa-Sharpe Agar (“MRS”).

For isolation of Escherichia coli, the resulting agar plates were incubated at 37° C. in a humidified room-air (i.e. aerobic) environment. For isolation of Lactobacillus species, the resulting agar plates were incubated at 37° C. in an airtight jar containing an AnaeroPack (Mitisubishi Gas Chemical America). When colonies (and their media-specific phenotypes, as in halos on MacConkey agar) were visible after approximately 12-24 hours, candidate colonies were picked and manually streaked for isolation on the same agar media as the source plates. After incubation of these agar plates under the same conditions as the original plates, colonies isolated by at least a growth-free zone of 3 mm were picked and grown in sterile 16 mm×160 mm glass culture tubes containing 1 mL of liquid growth media. For Escherichia coli, the liquid media was Lysogen Broth containing 0.2% w/v glucose and 5 g/L of NaCl. For Lactobacillus strain, the liquid media was De Man-Rogosa-Sharpe media, and the tubes were sealed with rubber stoppers. Cultures were grown with shaking in a 37° C. orbital shaker until they became turbid, at which point 0.5 mL of culture was transferred to a screw-top cryostorage vial containing glycerol (0.05 mL for Lactobacillus and 0.1 mL for Escherichia coli), mixed by trituration, and stored at −80° C.

Strain Identification.

The cells in the remaining liquid cultures were transferred to sterile 1.7 mL microcentrifuge tubes and collected by centrifugation at 14,000 RPM for 1 minute. The supernatant was aspirated, discarded, and the cell pellet was resuspended in 50 μL of sterile deionized water.

Ribosomal 16S DNA was amplified by PCR using primers specific to the V3-V4 region using Phusion DNA polymerase (New England Biolabs). The water-suspended cells were used directly as template material, in 10-fold dilutions starting with the concentrated cell slurry. Total reaction volumes were 25 μL. After cycling, 5 μL of each reaction was analyzed by elution on a 1% w/v 0.5×TAE agarose gel with SYBR Safe DNA stain (Invitrogen). Reactions exhibiting an approximately 450 bp product without substantial primer dimers or non-specific product bands were identified, and the remaining 20 μL of PCR solution was purified using a silica spin-column kit (Invitrogen or New England Biolabs). The resulting DNA was quantified by UV-vis spectrophotometry and sequenced by Sanger dideoxy sequencing by a service provider (Eton).

On receipt of the sequencing results, chromatograms were manually inspected for strong, well-separated fluorescence peaks. The DNA basecalls from these chromatograms were compiled and aligned to the NCBI non-redundant (“nr”) database using BLAST. Strains were tentatively identified by the preponderance of genus/species genomes with >=97% identity to the sample sequence.

To confirm the strain identities and uniquely identify the strains from each other, regions of highly variable genes specific to the identity assigned by 16S sequencing were amplified and sequenced. For Escherichia coli, the gyrB and fumC sequence alleles were used. Table 2 summarizes the strain identities of different E. coli strains we have isolated.

TABLE 2

| strain | source | adk | fumC | gyrB | icd | mdh | purA | recA | #matching | ST complex |
|---|---|---|---|---|---|---|---|---|---|---|
| AZ39 | mouse | 13 | 52 | 17 | 156 | 14 | 17 | 25 | 0 | |
| AZ61 | human | | 335 | 10 | | | | | 1 | |
| AZ73 | human | | 100 | 17 | | | | | 8 | |
| AZ74 | human | | 24 | 9 | | | | | 93 | ST73 |
| AZ75 | human | | 38 | 19 | | | | | 93 | ST95 |
| AZ76 | human | | 11 | 4 | | | | | 608 | multiple |

Precursor Strain Engineering.

E. coli MG1655 (a laboratory-adapted strain) was transformed with pSIM18 which encodes the lambda-Red recombination genes bet, exo, and gam under the control of a 42° C.-inducible promoter, confers resistance to hygromycin, and can be lost from the containing strain by unselected growth at 37° C. or higher. This strain, MG1655(pSIM18), was used to create numerous further derivative strains.

To confer constitutive expression of the marker gene green fluorescent protein (GFP), we used a sequence fusing the gfpmut2 gene to the promoter region for the E. coli gene rp1N and containing the aph kanamycin resistance-conferring gene from the plasmid collection described by Zaslaver et. al. Nat Methods. 2006 August; 3(8):623-8. This sequence was amplified by PCR incorporating 30-40 nucleotide overhangs homologous to the bacteriophage lambda attachment site, attB. This PCR fragment was used for recombination with MG1655(pSIM18) to produce strain MG1655 attB::[aph Prp1N-gfpmut2] KmR which fluoresced green with blue-light illumination and the sequence of which was confirmed by Sanger dideoxy sequencing (SEQ ID NO:1). A culture of these cells was lysed with bacteriophage P1vir to produce a transducing phage P1vir(attB::[aph Prp1N-gfpmut2] KmR).

To confer expression of a heterologous gene, we constructed an expression cassette containing the hybrid Ptrc promoter sequence, an [aph PrhaB-ccdB] counterselection cassette, and an chloramphenicol acetyltransferase (cat) gene. The cat gene was flanked by two FRT recombination sites for subsequent excision if desired. This cassette was amplified by PCR to incorporate 30-40 nucleotide overhangs homologous to the yfgG-yfgH intergenic region and introduced to MG1655(pSIM18) followed by plasmid curing and Sanger dideoxy sequencing to produce MG1655(yfgG::[Ptrc-[aph Prha-ccdB] FRT-cat-FRT]) (SEQ ID NO:2). This strain was then re-transformed with pSIM18 and a PCR product containing a codon-optimized variant of the predicted sequence of Lactobacillus salivarius JCM1046 (GenBank ACL98194.1) and extensions homologous to the ORF location of the Ptrc expression region. The subsequent strain was cured of pSIM18, the sequence confirmed by PCR and Sanger dideoxy sequencing, and subsequently stored as MG1655(yfgG::[Ptrc-BSH FRT-cat-FRT]) (SEQ ID NO:3). Activity of the BSH gene was confirmed by cultivation as single isolated on Lennox LB agar media containing CaCl₂ and sodium taurodeoxycholate, which resulted in colonies surrounded by a white halo in the agar, as well as thin-layer chromatography of an in vitro sodium taurocholate deconjugation reaction containing the same strain of cells partially lysed by freeze-thaw cycling at −20° C. A culture of these cells was lysed with bacteriophage P1vir to produce a transducing phage P1vir(yfgG::[Ptrc-BSH FRT-cat-FRT] CmR).

Transfer of Genetic Blocks to M-ACT Strain.

Strain AZ-39 was isolated by the method described above from fecal samples of male wild-type, conventionally-raised C57B6 mice maintained in a vivarium colony at UCSD. This strain was sensitive to kanamycin, chloramphenicol, carbenicillin, hygromycin, trimethoprim and ciprofloxacin. The genomic sequence of AZ-39 was determined from data acquired from its extracted DNA on a long-read high-throughput sequencing instrument (Pacific Biosciences). AZ-39 exhibited no hemolysin or other known toxin genes (e.g., Shiga-like toxin, fragilysin) in its genomic sequence.

AZ-39 was subsequently transduced to kanamycin resistance and chloramphenicol resistance using P1vir(attB::[aph Prp1N-gfpmut2] KmR) to produce strain AZ-51 (AZ39 GFP+) and subsequently with P1vir(yfgG::[Ptrc-BSH FRT-cat-FRT] CmR) to produce strain AZ-52 (AZ-39 GFP+ BSH+). Both AZ-51 and AZ-52 fluoresced green under blue light illumination and AZ-52, but not AZ-51, produced halos when grown for single isolates on solid agar media containing sodium taurodeoxycholate.

Colonization of Wild-Type, Conventionally-Raised Mice.

Strains AZ-51 and AZ-52 were streaked for single isolates from their respective glycerol stocks. Individual colonies from these streaks were tested for correct BSH activity phenotypes, and subsequently inoculated into 1 mL cultures of Lennox LB+1 mM MgCl₂, with kanamycin (25 μg/mL) for AZ-51 and chloramphenicol (10 μg/mL) for AZ-52. These cultures were grown for 12 hours at 37° C. with shaking at 215 RPM. Each culture was subsequently back-diluted 1000-fold into 50 mL of the same media in 500 mL baffled culture flasks additionally supplemented with 1 mM MgCl₂ and 0.5x M buffer as described by Studier (2005) Protein Purification & Expression. These cultures were grown for a further 12 hours at 37° C. with shaking at 215 RPM. At the end of the growth period, the absorbance of the cultures at 600 nm with a 1 cm path length was routinely between 10.0 and 20.0 (accounting for dilution prior to measurement). Cells were collected from a measured volume of liquid culture and resuspended in cold, sterile 1×PBS to a cell density of 5×10¹⁰/mL, assuming a cell density of 1×10⁹ cells/mL/OD600 in the original culture liquid. This solution was subsequently kept on ice. Within an hour, 0.2 mL of this cell suspension was delivered to wild-type, conventionally-raised C57B6 mice by oral gavage. Mice were subsequently returned to their housing. Cages were changed twice per week for the first week after gavage, weekly for the following 2 weeks, and biweekly thereafter.

Colonization status was monitored by collecting, homogenizing, and plating fresh fecal samples as described above, with the except that once homogenized, fecal samples were additionally plated on Lennox LB agar media containing either kanamycin (25 μg/mL) or chloramphenicol (10 μg/mL). Imaging data were recorded by photographing the resulting plates after outgrowth with blue light transillumination. Retention of BSH activity was tested by restreaking clones from antibiotic-free plates (e.g. EMB Sucrose, MacConkey Lactose) onto Lennox LB agar media containing CaCl₂ and TDCA.

### Example 2

### Colonization of the Murine Gut with Re-Introduced and Engineered Commensal Bacteria

In this example, a strain of E. coli was isolated from C57Bl/6 mice, engineered to express GFP and a bile salt hydrolase gene from Lactobacillus, then reintroduced to C57Bl/6 mice.

### Introduction

Dysbiosis has been associated with changes in social, communicative, stress-related, and cognitive behavior in murine models (11, 12). Human studies have linked perturbations in the gut microbiome and autism spectrum disorders (13), major depression (14), and Parkinson's disease (12). There is growing evidence that microbiome-neuroimmune interactions can mediate behavioral and physiological abnormalities observed in murine models, specifically through global changes in brain transcriptome, altered microglial maturation and function, and integrity of the blood brain barrier (BBB) (1, 15). However, it's not clear what agents, and through what mechanisms, these effects are mediated. Following, we review the link between the gut microbiome, bile acids (BAs), neuroinflammation, and behavioral dysfunction.

Microbiome and Bile Acid Metabolism:

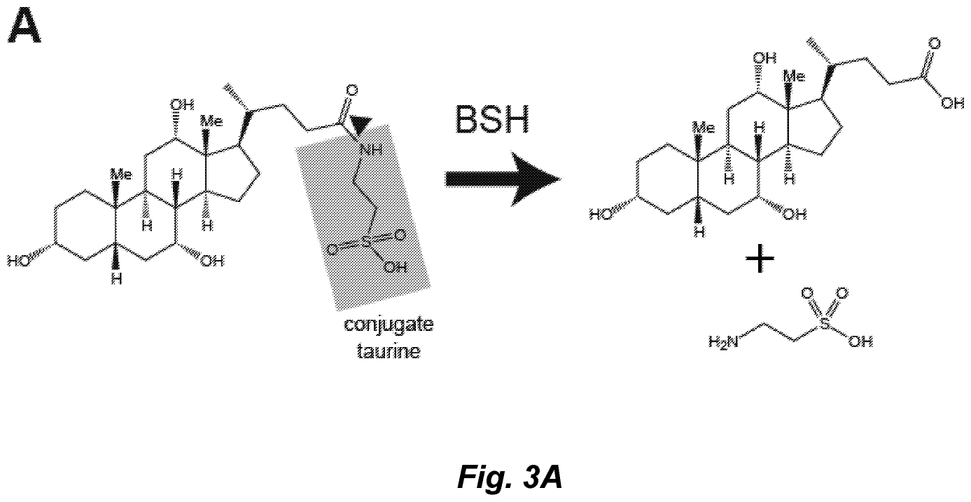

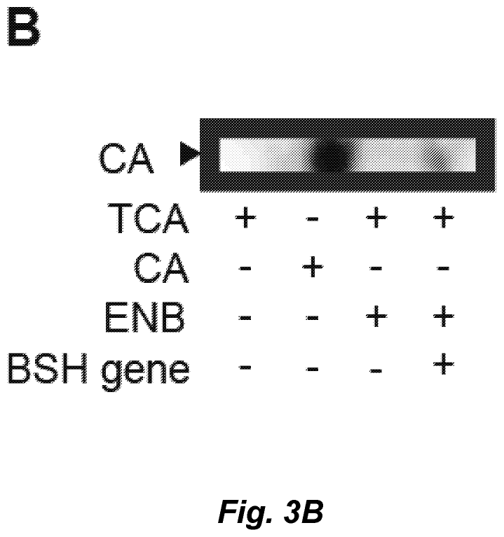

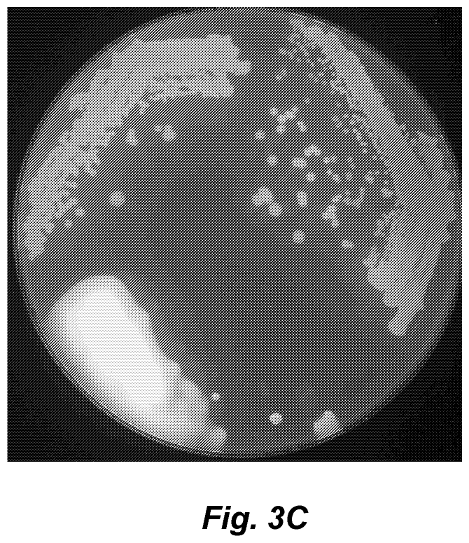

Microbial deconjugation of BAs (i.e. removal of glycine or taurine; FIG. 3A) by bile salt hydrolase (BSH) plays a critical role in host physiology. Deconjugation prevents BA reuptake from the small intestine and leads to their conversion into secondary BAs in the colon (16). Although much of this hydrophobic pool of secondary BAs is excreted, enough is absorbed through passive diffusion to change the serum BA pool and act as signaling molecules (17). BA signaling is mediated by two known receptors: the farnesoid X receptor (FXRα) (18) and the G protein-coupled BA receptor 1 (TGR5) (19). In metagenomic studies of human populations and mouse models, BSH has been identified as potentially protective against obesity, dysmetabolism, and a host of other physiological disturbances (20).

Bile Acids and Neuroinflammation:

BAs can modulate neuroinflammation. Both FXRα and TGR5 receptors are found in brain tissue, including microglia and neurons. Ursodeoxycholic acid (UDCA), a secondary BA created by bacteria, and its hepatic taurine conjugate (TUDCA), are immunomodulatory agents that affect microglia. UDCA inhibits the production of the pro-inflammatory cytokine IL-1β and nitric oxide (NO), and can counteract a neurotoxin's effects on neuronal death and synaptic changes in vitro (21, 22). In mouse models of neuropathologies, TUDCA reduced microglial activation, decreased inflammatory cytokines, and preserved neuronal integrity (2, 23). Although most studies on BAs and neuroinflammation have used UDCA or its conjugates, it's not clear whether other BAs have similar effects. The UDCA immunomodulatory effects are mediated through the TGR5 receptor (5). In fact, a TGR5 agonist also reduced microglia activation and proliferation and reduced proinflammatory cytokines (10). However, other receptors by which BAs can affect neuroinflammation have also been proposed (4).

High Fat Diet, Obesity, and Neuroinflammation:

High-fat diet (HFD) consumption causes neuronal leptin and insulin resistance, disrupting homeostatic signals and creating a positive energy balance (24, 25). Similar to peripheral metabolic tissues (e.g. liver, adipose tissue), neuronal resistance to these signals has been linked to activation of the inflammatory signaling cascade (26-28). In the brain, HFD feeding is accompanied by expression of proinflammatory cytokines, gliosis, alteration in vasculature, and disruption of BBB permeability (29). In the hippocampus specifically, there is increased IL-10, IL-6, and TNF-α mRNA and protein expression, as well as increased microgliosis (29-32).

High Fat Diet and Behavioral Dysfunction:

In rodents, there is good evidence that HFD-induced neuroinflammation can cause memory dysfunction and anxiety, especially with longer duration exposures. Mice on a HFD (60% kcal from fat) for at least 16 weeks clearly displayed impaired recognition memory (as assessed by the novel object recognition test), whereas mice with a 5-week exposure to HFD did not (33-35). Prolonged exposure to HFD (i.e. >20 weeks) also resulted in impaired spatial memory and learning (29, 31, 36-38), and increased anxiety levels as assessed by the marble-burying test, elevated plus-maze, and open field test (33, 35, 39). These cognitive deficits were accompanied by neuroinflammation as determined by increased levels of IL-6 and TNF-α, and microglia activation.

Limitations in Functional Manipulation of the Gut Microbiome:

The gut microbiome can modulate a variety of host physiological processes, but it's not clear how its effects are mediated. Furthermore, it is unclear whether most interventions that target microbiome composition (e.g. probiotics—live bacteria that are believed to provide health benefits) have a detectable impact on the gut microbiome (40), or are robust to the interpersonal diversity and plasticity of the microbiome in conventionally raised wild-type (CR-WT) hosts (e.g. humans) (41). To develop a better mechanistic understanding and more effective microbiome-mediated therapies, a different approach stressing a functional modulation of the gut microbiome is necessary. Current strategies to address this problem have focused on creating engineered bacteria from laboratory adapted bacterial strains. However, these efforts have been labor-intensive, expensive, and disappointing since these bacteria do not colonize CR-WT hosts (41).

Herein, we present methods and compositions that employ “knock-in” functions into the gut microbiome and investigate their effects on the luminal ecology, the flux of metabolites and nutrients, and physiology in CR-WT hosts. While using this tool to investigate the effects of luminal BA biotransformation on host metabolism, we noted that mice that had BSH knocked in had remarkable overt differences in their behavior.

Current Paradigm and its Limitations:

New tools are necessary to understand whether a biochemical function determined by metagenomics, metabolomics, and/or metatranscriptomics can convey, or disrupt, a phenotype. Since gut microflora can sense and manipulate the luminal environment, they have become an attractive avenue for engineered cell-based therapeutics. However, the inability of engineered laboratory-adapted bacteria to colonize CR-WT hosts in useful numbers and/or for a meaningful period of time and/or to achieve physiological change in CR-WT mammals has thus far limited their use in mechanistic and therapeutic studies (41). Long-term functional manipulation of the gut microbiome of CR-WT hosts with engineered bacteria remains elusive. It is too difficult for probiotics (whether engineered or not) that are not adapted to the host to compete with the microflora already present in the lumen. There are multiple barriers to their survival in the luminal environment, including those from the host (e.g. peristalsis, innate and adaptive immunity) and other native microorganisms (e.g. competition, niche availability) (42).

It is well recognized that lab strains are not suitable vectors for function delivery. To address this problem, some research groups have developed tools to manipulate families of bacteria that are commonly found in the gut microbiome, specifically Bifidobacteria, Lactococci, and Lactobacilli. By engineering hundreds of presumed commensal species and systematically feeding them to CR-WT mice, these labs have been searching for long-term colonizers. Despite the use of tremendous resources, this method has not yet produced a colonizer in CR-WT hosts. Furthermore, developing colonizing bacteria with this method would likely require similar prodigious resources for new hosts (e.g. transgenic mice, human hosts) and new genetic functions, making these methods out of the reach of resource-limited labs.

Our New Paradigm:

We have developed a technique that has advanced our ability to use engineered bacteria to effectively change physiology in a CR-WT host. We have been able to accomplish this by identifying tractable native/commensal bacteria derived from the CR-WT host, genetically modifying them with genes that impart a beneficial function, and then re-introducing the engineered native bacteria (ENB) to the CR-WT host. Hence, ENBs are already adapted to the luminal environment. The reluctance to use this method in the past is driven by the assumption that it is difficult to culture and modify native bacteria. We have used a native E. coli isolated from mouse feces. Bacterial engineering of E. coli can be performed by practically any lab with very little resources. Though E. coli are common native bacteria, many investigators have assumed that they are poor colonizers because of disappointing experiences with laboratory adapted strains. However, we have been successful creating engineered bacteria (including E. coli) that can:

(1) colonize hosts maintained on different diets for months (e.g., at least 140 days stable colonization) after a single gavage.

(2) express a gene in the gut (e.g., “knock-in” BSH) which can alter luminal BAs.

(3) change the serum metabolites in a predictable fashion (e.g. decrease serum conjugated BAs).

(4) change host core metabolic processes.

(5) change the behavior of the animal.

ENBs expand our ability to functionally manipulate the gut microbiome and to perform more mechanistic microbiome studies, including identifying mediators of the microbiome-gut-brain axis. Specifically, we can determine whether BA deconjugation can affect host behavior and cognition. Moreover, ENBs can be used as therapeutic agents in CR-WT hosts, including humans.

### Results

In Vitro Metabolite Modification Using ENB:

Native microflora provide a wealthy reservoir for vectors that are tractable and can colonize the host. By culturing the feces of C57BL/6 mice, we identified a genetically tractable E. coli, EcAZ. We converted EcAZ to an ENB expressing BSH by introducing two genes into the chromosome of this strain, a Prp1N-GFPmut2 cassette and a Ptrc cassette containing a single-step introduction site for heterologous genes (43). EcAZ was modified to express codon-optimized L. sallivarius BSH (EcAZ^{BSH+}). BSH cleaves the taurine from the side chain of the BA core sterol (FIG. 3A). This new ENB hydrolyzes taurocholic acid (TCA) into cholic acid (CA) in an in vitro reaction (thin-layer chromatography; FIG. 3B). When grown on agar plates containing 10 mM TCA, a white halo of the less soluble hydrolysis product (CA) forms around the BSH-positive strain but not the parental BSH-negative strain (FIG. 3C).

ENB can Colonize the Gut of CR-WT Mice after a Single Gavage:

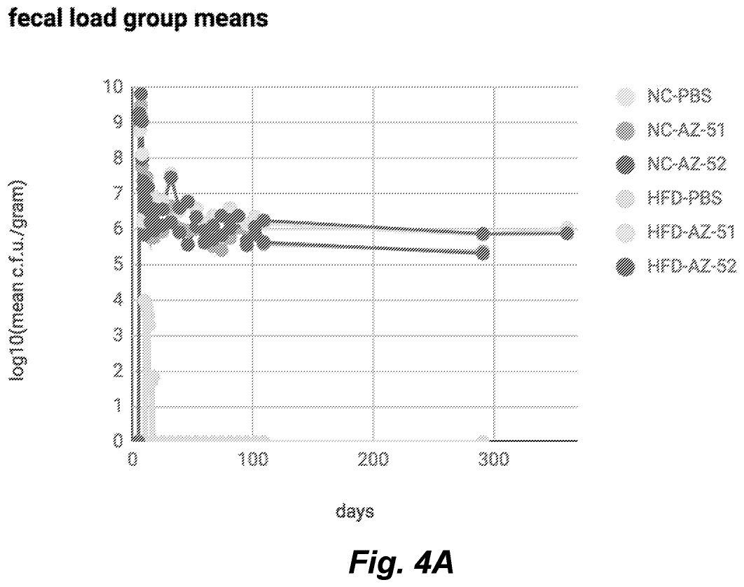

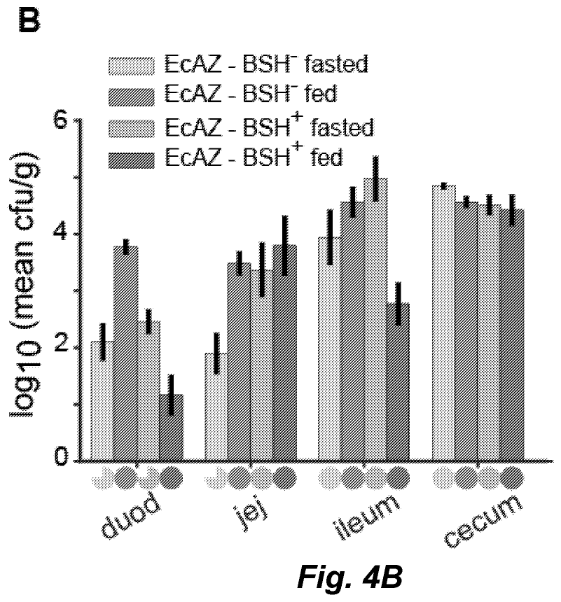

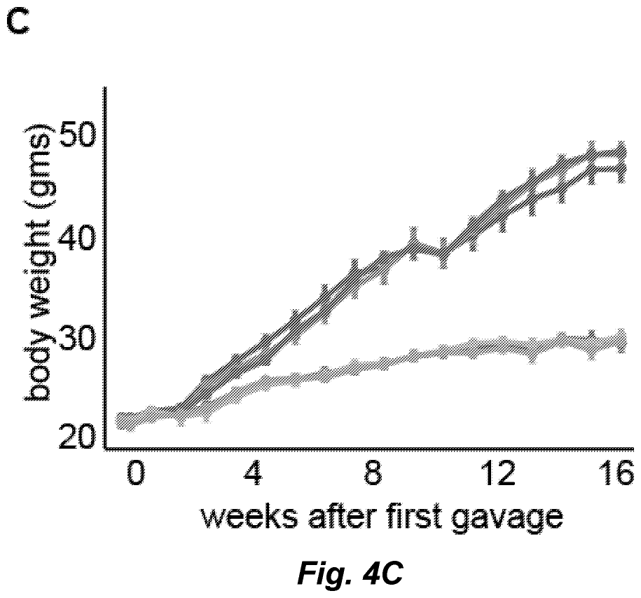

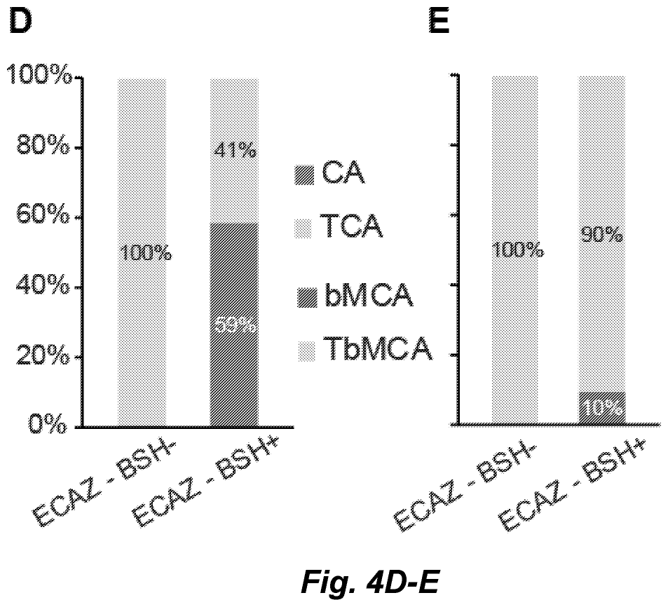

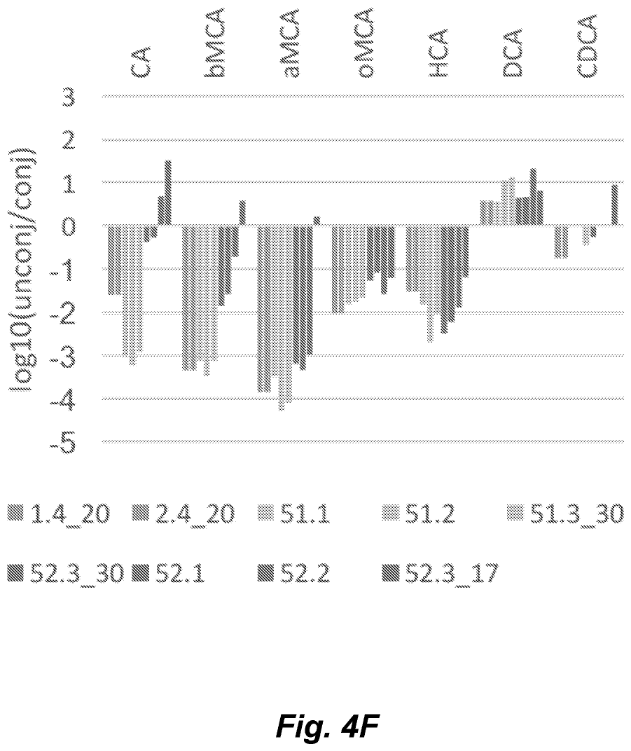

Ten-week old CR-WT C57BL/6 mice received a single gavage of stationary-phase cultures of GFP+ EcAZ (EcAZ^{BSH+}/EcAZ^{BSH-}). Stool samples were collected from each mouse independently and were plated on agar media. The number of GFP+ colony-forming units per gram of stool remained stable at −10⁶ for more than 20 weeks (data presented up to 15 weeks; FIG. 4A) in both normal chow diet (NCD) and HFD feeding conditions. The addition of BSH gene did not affect the ENB's ability to colonize the gut (FIG. 4A). After 8 weeks, some of the mice were euthanized and organs collected and plated to determine extent of colonization. EcAZ colonized the entire GI tract of treated mice with the highest concentration in the terminal ileum and cecum (FIG. 4B). It did not colonize spleen, lung, heart, or liver of the mice. All the mice appeared healthy and were indistinguishable from each other. Colonization with either EcAZ^{BSH+} or EcAZ^{BSH-} did not affect body weight (FIG. 4C). In germ-free (GF) mice, EcAZ^{BSH+} deconjugated BAs whereas EcAZ^{BSH-} did not (FIG. 4D, 4E).

ENB can Perform BA Biotransformation in the Gut and Affect Serum BA Pool:

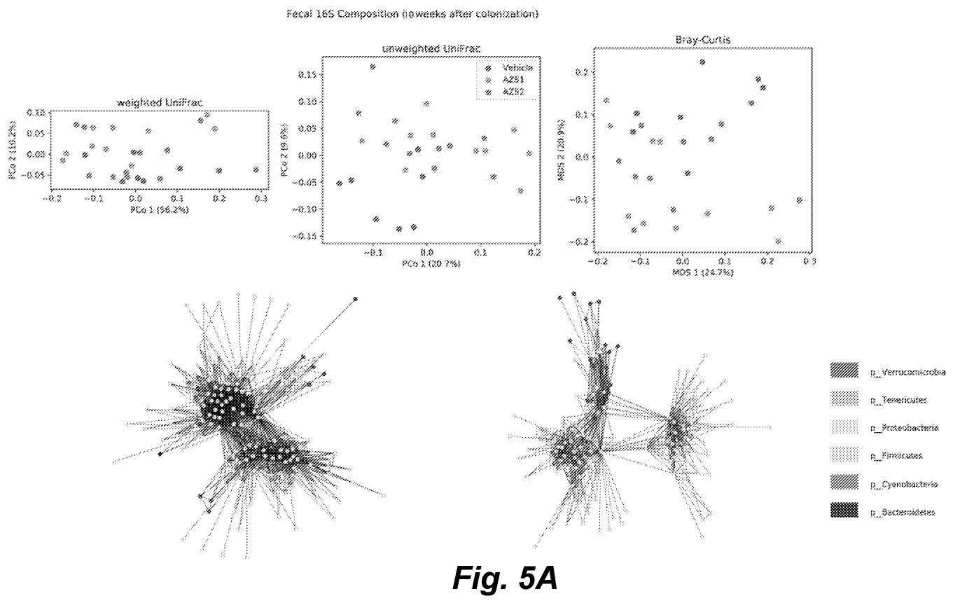

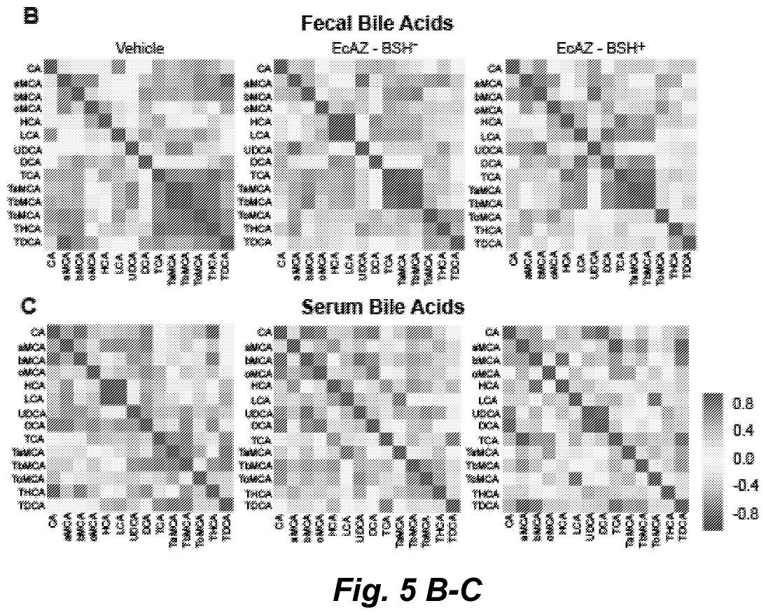

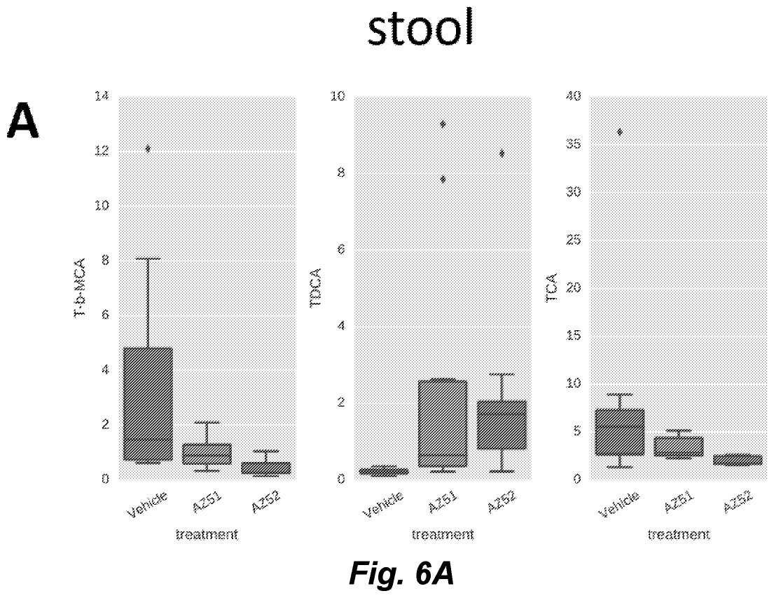

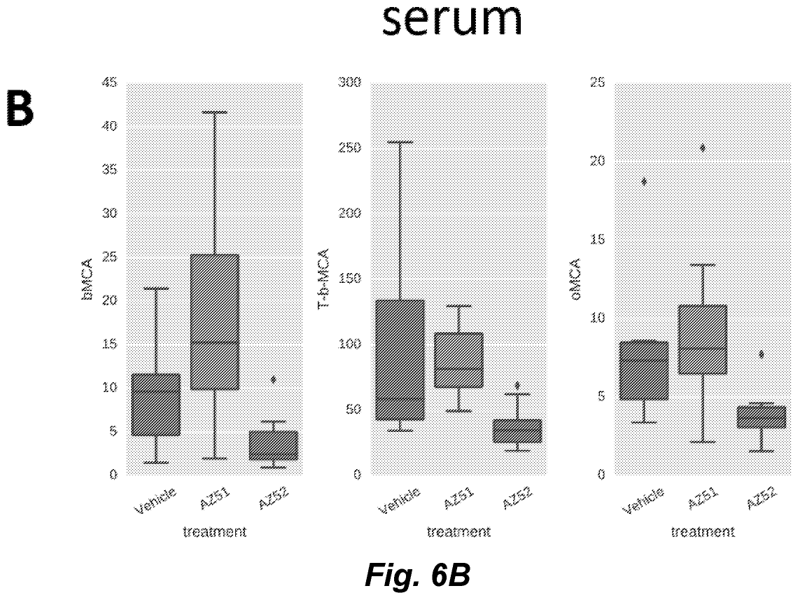

Analysis of the stool showed that the addition of ENBs did not cause major shifts in microbiome composition (FIG. 5A). Targeted metabolomics confirmed that EcAZ^{BSH+} deconjugated primary BAs, which led to metametabolomic changes in BA pool in the lumen (FIG. 5B). Although many fecal BAs were different between groups, the changes were unpredictable. However, the changes in serum BA composition were far more predictable. There was a global decrease in conjugated BAs (FIG. 5C), Hence ENBs can be used to change the host BA composition.

ENB can Induce a Physiological and Behavioral Change:

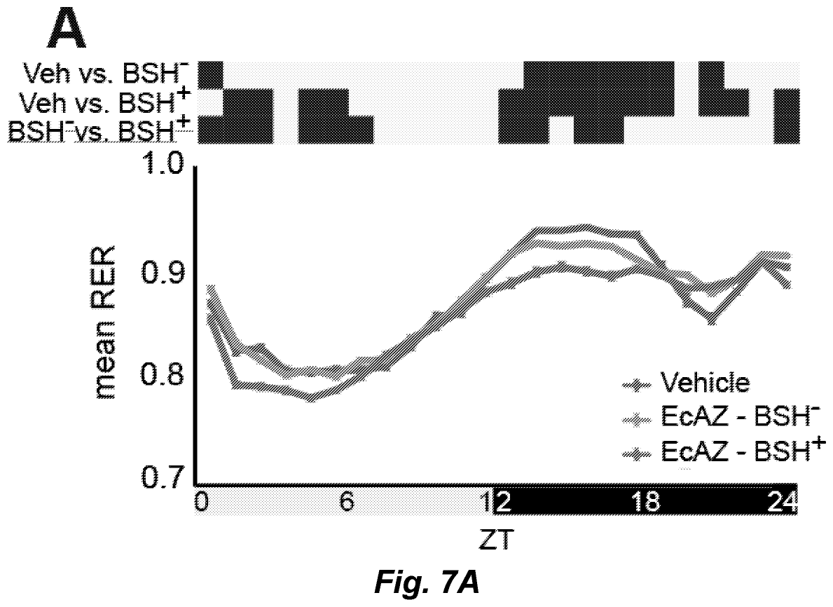

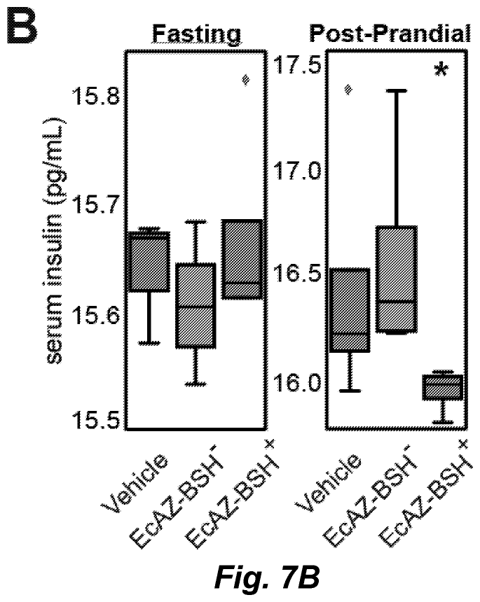

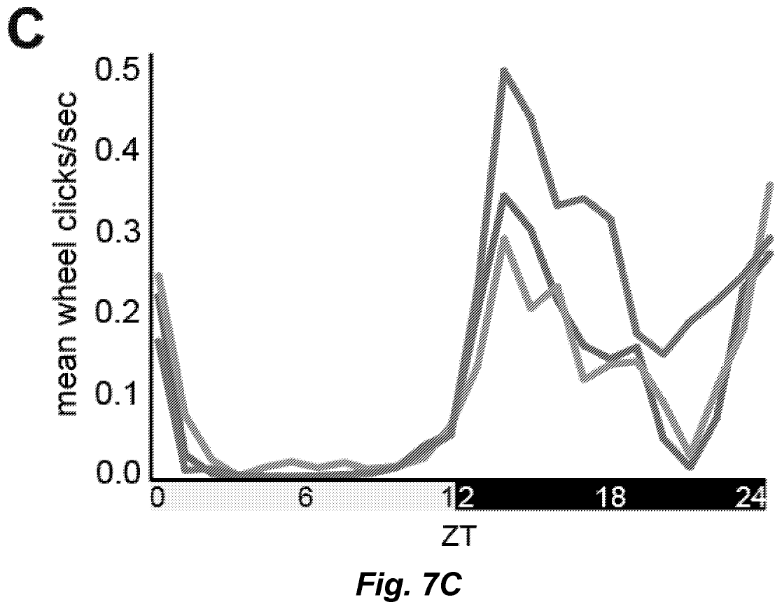

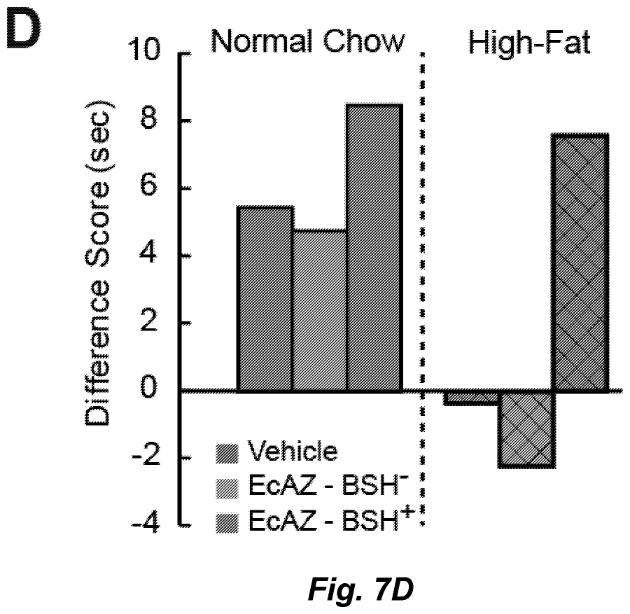

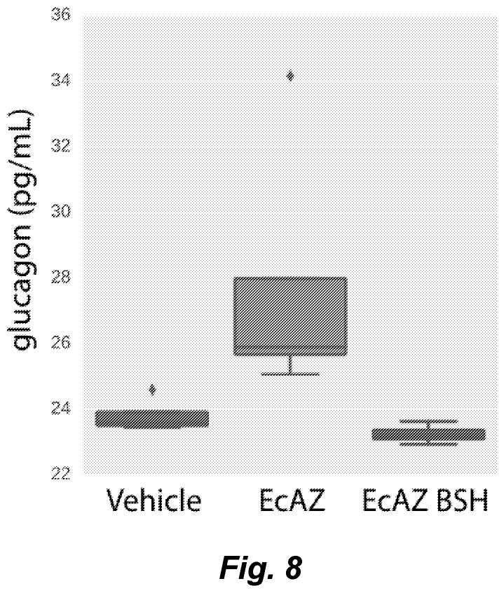

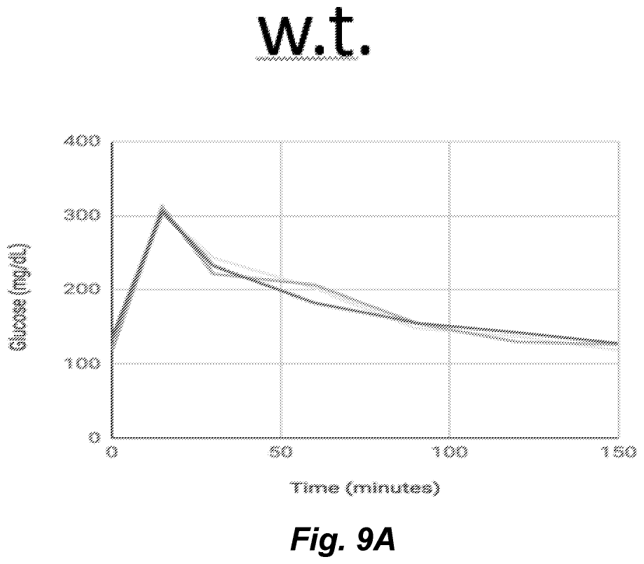

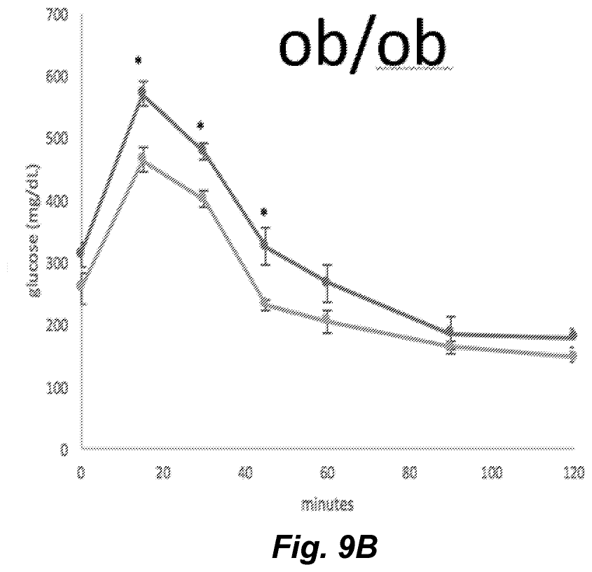

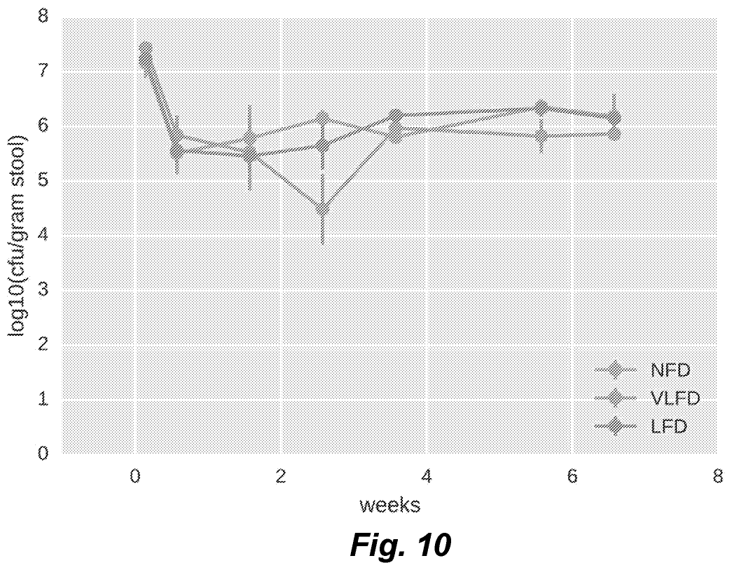

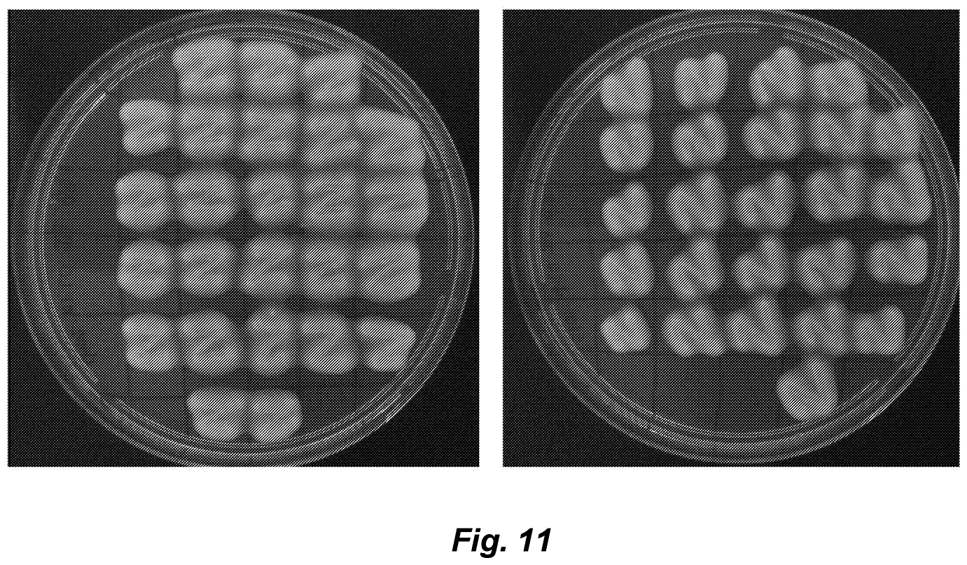

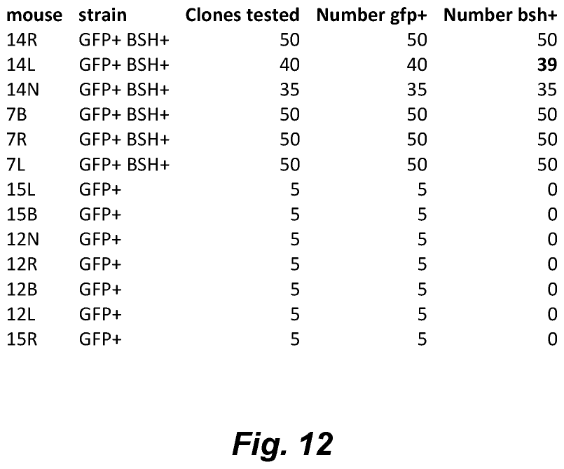

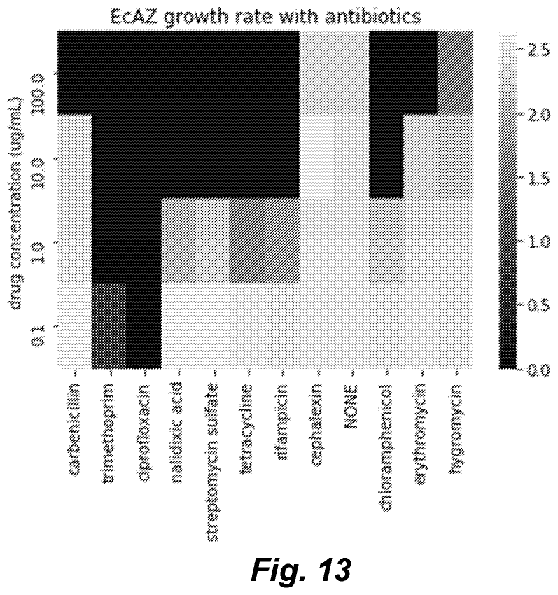

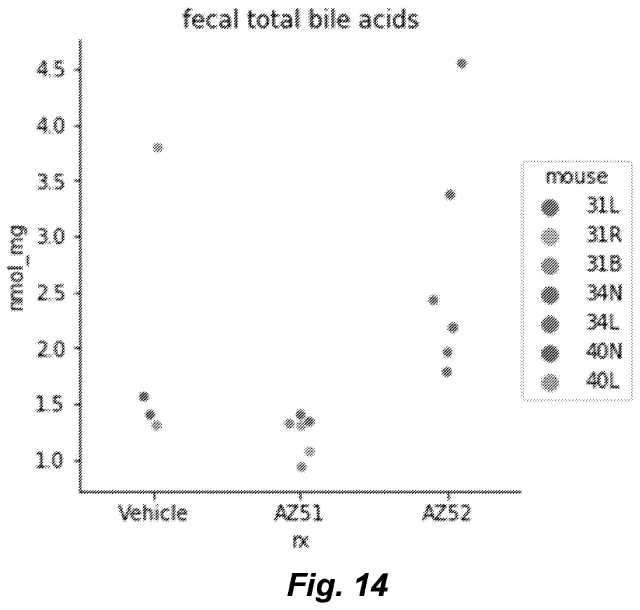

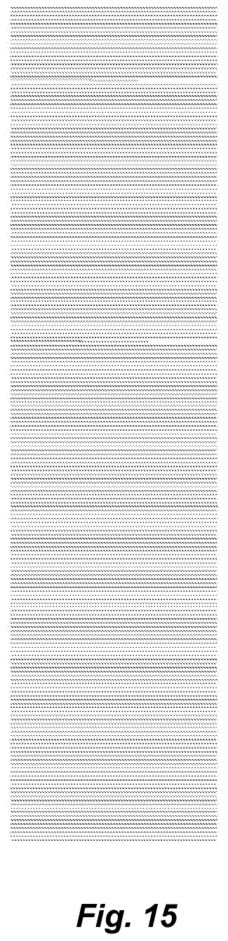

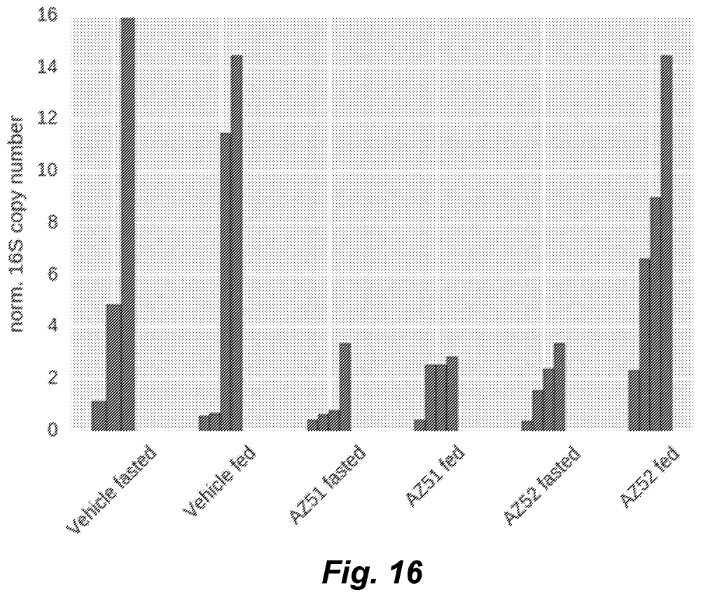

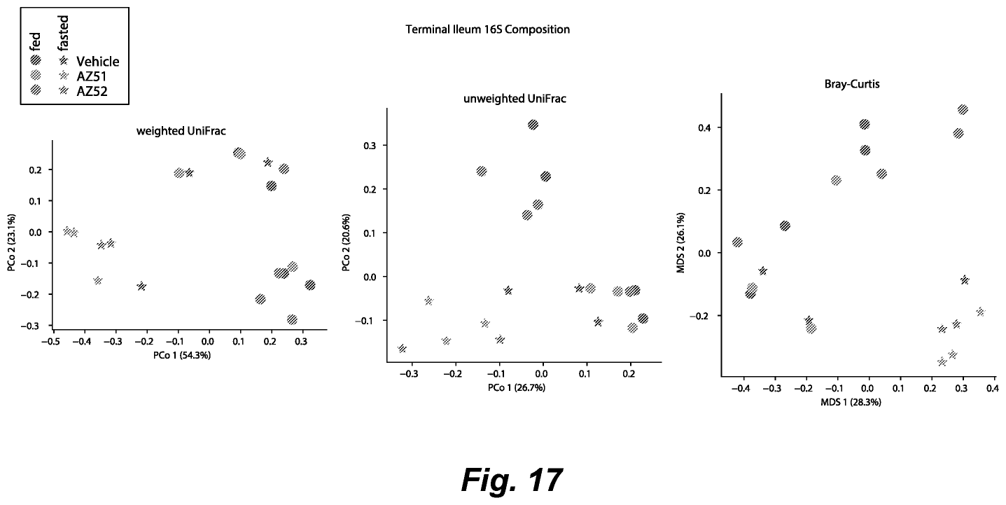

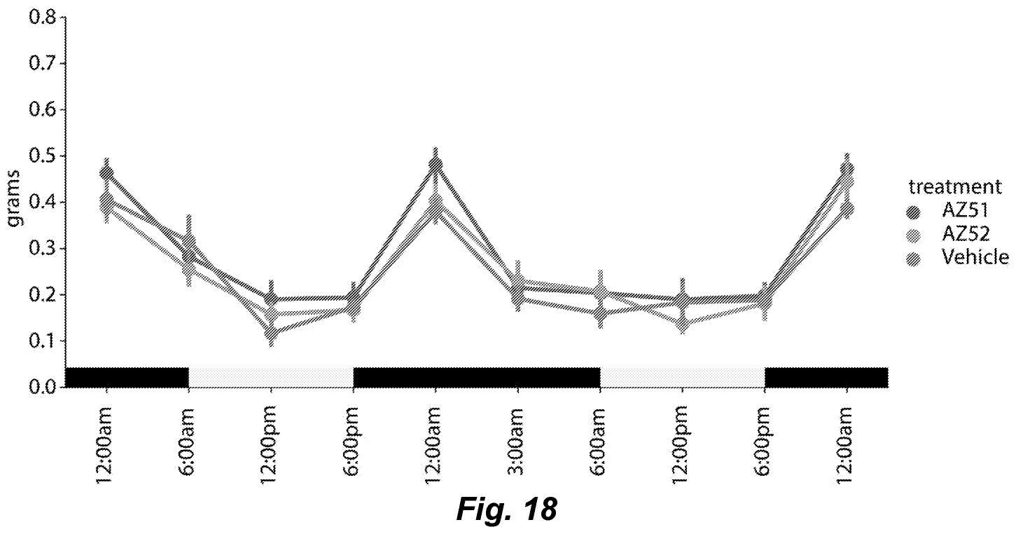

Metabolic cage assessment of EcAZ^{BSH+} mice revealed a distinct metabolic profile, characterized by a low respiratory exchange rate (RER), suggesting increased fatty acid oxidation (FIG. 7A). BA signaling also influences glucose homeostasis; EcAZ^{BSH+} treated mice had significantly lower post-prandial insulin levels (FIG. 7B, right). To quantify our observation of behavioral differences between mice in these conditions, we performed behavioral and cognitive experiments. EcAZ^{BSH+} mice spent 50% more time on the running wheel compared to the two controls (FIG. 7C). EcAZ^{BSH+} also performed marginally better in a pilot novel object recognition test (FIG. 7D). While mice on HFD performed poorly on this cognitive test after 20 weeks, EcAZ^{BSH+} treated HFD mice did not have any difficulty (FIG. 7D). Hence, expression of a single gene, BSH, in the microbiome induces metabolic, behavioral, and possibly cognitive change in the CR-WT host.

### Experimental Methods and Design

### Luminal BA Modification by ENB Affects Cognition in CR-WT Mice.

Our data show that high fat diet (HFD) consumption causes impaired novel object recognition. However, HFD mice treated with EcAZ^{BSH+} performed as well as normal chow diet (NCD) mice did, suggesting that bacterial BA deconjugation affects host cognition.

Methodology:

ENB colonization and In vivo Assessment. Eight-week-old WT male C57BL/6 mice (72 mice total) are used for this experiment. Only male mice are used since they are susceptible to diet-induced obesity whereas female C57BL/6 mice do not become obese when given a HFD (44). Weight and food consumption are monitored for the duration of the experiment. After 2 weeks, 10-week-old mice receive a single gavage of PBS, EcAZ^{GFP+/BSH-} or EcAZ^{GFP+/BSH+} (24 mice per group). Using stool cultures, colonization and BSH activity is monitored throughout the experiment. After 2 weeks, half of each group is switched from NCD to HFD (12 mice per condition; LabDiet 58Y1; 18% protein, 61% fat, 21% carbohydrates). Behavioral testing begins 20 weeks later, when cognitive deficits associated with HFD are present (34).

In Vivo Assessment of Luminal and Serum BA Profile.

Twenty-two weeks after gavage (20 weeks after diet change), blood (submandibular) is collected from all mice to use for targeted BA metabolomics. The effect of ENBs on the targeted fecal and serum BA pool is assessed by liquid chromatography-coupled mass spectrometry (LC-MS/MS).

Behavioral Testing:

Equipment used for behavioral testing is autoclavable and suitable for high barrier facility. This is to minimize environmental factors that can affect the composition of the microbiome. In addition, the order of testing is designed to minimize confounds as the tests progress. Milder tests are performed before tests that may cause more than momentary pain or distress such as tail suspension. Tests are performed in the order below separated by 3-5 days.

Open Field Test.

This is a test of “emotionality” used to measure anxiety-like responses of rodents exposed to stressful environmental stimuli (brightly illuminated open spaces) as well as to capture spontaneous activity measures. Each animal is placed in the center of an open arena (45) and several behavioral parameters (distance traveled, velocity, center time, frequency in center, rearing, grooming) are recorded during a 30-minute observation period and analyzed.

Hanging Wire Test.

The hanging wire test allows for the assessment of grip strength and motor coordination (46). Mice are held so that only their forelimbs contact an elevated metal bar held parallel to the table by a large ring stand and let go to hang. Time to fall and a score based on hanging strategy is assigned.

Novel Object Recognition Test.

This test assays recognition memory while leaving the spatial location of the objects intact (47-49). The basic principal is that animals explore novel environments and that with repeated exposure decreased exploration ensues (i.e., habituation) and then a novel object is explored preferentially (dishabituation) because it differs from what the animal remembers (50-52). Behavior is video recorded and then scored for contacts (touching with nose or nose pointing at object and within 0.5 cm of object).

Marble Burying Test.

The marble burying test is used to assess anxiety-like (53) and obsessive-compulsive-like behavior (54) capitalizing on a species-typical behavior of digging (55). Mice are placed individually in standard mouse cages containing bedding that is 5 cm in depth, with 20 evenly spaced marbles for 30 min, after which mice are removed and the number of marbles buried (at least ⅔ covered by bedding) is determined.

Barnes Maze Test of Spatial Memory.

The Barnes maze test is a spatial learning and memory test that involves the use of distal visual cues to escape a brightly lit circular field (56-58). Each session is videotaped and scored by an experimenter blind to the experimental condition of the mouse and analyzed to assess distance traveled, velocity of movement and for path analyses.

Tail Suspension Test.

The tail suspension test is a classic test for examining helplessness/depressive-like behavior in mice (59, 60). Each mouse is suspended from its tail using adhesive tape on a metal bar located 30 cm above a flat surface for 6 minutes. Immobility is quantified by measuring the amount of time when no whole-body movement is observed. Increased time spent immobile is indicative of increased depressive-like behavior.

Statistical Analyses.

The StatView (version 5.0.1; SAS Institute Inc.) is used. Data from each behavioral test are assessed using repeated measures ANOVA with the between-subject factors group and the within-subject factor time or trial depending on the test. Fisher's protected least significant difference (PLSD) post-hoc tests are used when warranted by significant main effects or significant interactions between these effects.

Post-Mortem Assessment of BA Signaling.

After behavioral phenotyping, fasting serum and feces are collected from each mouse. Mice are terminated using CO2 asphyxiation followed by decapitation. Brain, lung, heart, liver, spleen, and different segments of the gut are homogenized in PBS and then qPCR for the EcAZ-specific genomic island mRNAs (61), GFPmut2, and BSH, as well as culture tissue are performed, to determine the site(s) of EcAZ colonization. BAs and short-chain fatty acids are measured in the serum, feces, liver, brain, and the terminal ileum and cecal content. Host RNA is extracted from the terminal ileum, cecum, liver, and hippocampus. For half of the mice (6 per condition), RNA-seq (Illumina HiSeq SE50) is performed. Transcriptomic results of key genes are confirmed with qRT-PCR in the other half of mice (6 per condition).

Increased bacterial BA deconjugation in the gut lumen leads to changes in terminal ileum BA pool. This activates both FXR and TGR5 pathways. Behavioral testing shows that there are cognitive differences between EcAZ^{GFP+/BSH+} mice and those from the PBS and EcAZ^{GFP+/BSH-} groups. This could from, e.g., reduced neuroinflammation, changes in the blood brain barrier permeability, direct action of the BAs on the neurons themselves, or a combination of factors.

### Luminal BA Modification by ENB Affects Microglia Gene Expression in CR-WT Mice.

HFD consumption can cause behavioral dysfunction through multiple mechanisms including disruption of BBB permeability, increased neuroinflammation, or altering neuronal transcriptome. Since BAs can modulate neuroinflammation, we determined whether ENBs affect microglia activation by changing the serum BA pool. We note that BSH activity can induce cognitive changes in absence of changes in microglia activation and, likewise can cause changes in microglia activation without inducing cognitive change.

Methodology: ENB Colonization, In Vivo Assessment, and Post-Mortem Assessment of BA Signaling.

Eight-week-old WT male C57BL/6 mice (48 mice total) are used. Animals are maintained, assessed, and colonized as described in Specific Aim 1 (SA1) except that there are 8 mice in each of the 6 conditions. All methods used to assess colonization and BSH activity employed in SA1 are used for these mice as well.

Microglia Isolation.

Microglia are isolated, as previously published (62), after the mice have been maintained on HFD for 24 weeks. Briefly, after a 6-hour fast, mice are deeply anaesthetized with CO2 and then quickly perfused intracardially with ice-cold DPBS. Whole brains are removed, the hippocampuses are dissected out, and half are used for immunostaining and microglia reconstruction (see below) and the other half used for microglia isolation and RNA-Seq. Hippocampal tissue is gently homogenized, filtered, and centrifuged. The pelleted homogenate is then resuspended in 37% isotonic Percoll, and then underlayed with 70% isotonic Percoll. Tubes are then centrifuged and the ones at the 37-70% Percoll interphase are recovered and washed with HBSS. Cells are then incubated in staining buffer on ice with CD16/CD32 blocking antibody, and then with anti-mouse CD11b-PE and CD45-Alexa488 antibodies. Sorting is performed with microglia identified as singlets, CD11b+CD45Low events, which encompassed >95% of all CD11b+ events. Isolated microglia are then pelleted and stored at −80° C. for downstream protocols.

RNA Isolation, Sequencing, and Analysis:

Total RNA is isolated from microglia homogenate by TRIzol. Library preparation and sequencing are performed by the UCSD sequencing core. Fastq files from RNA-Seq experiments are mapped to individual genome for the mouse strain of origin using STAR.

Immunostaining and Microglia Reconstructions.

Microglia are isolated and immunostained using previously published protocols (12). The dissected hippocampal tissue is fixed in 4% (w/v) paraformaldehyde. Using a vibratome, 50 mm sagittal sections are generated. Free-floating sections are stained with mouse anti-FXR NR1H4, rabbit anti-GPCR TGR5, and with goat anti-Ibal; and subsequently stained with anti-mouse IgG-AF647 and anti-rabbit IgG-AF546, and anti-goat IgG-AF488. Sections are mounted and imaged with a confocal microscope. Semi-automated reconstruction of microglia cell bodies and processes are performed. Twenty to sixty cells per animal are analyzed.

Cytokine Quantification.

TNF-α, IL-6, and other cytokines in tissue homogenates and serum are assessed using multiplex platform.

Increased bacterial BA deconjugation in the gut lumen leads to decreased neuroinflammation as determined by morphology (e.g., diameter of processes, number of branch points, total branch length) and cytokine production (either by multiplex/ELISA or transcription). In mice treated with EcAZ^{GFP+/BSH+}, a transcriptome program that is more similar to NCD control mice, whereas those with EcAZ^{GFP+/BSH-} are indistinguishable from vehicle treated HFD cohorts. Finally, the transcriptomic pathways indicate whether the changes induced by increase BA deconjugation are mediated by known BA receptors or some other undefined mechanism.

By using efficient gut colonizers that bypass the barriers that prevent most probiotics from colonizing the host, ENBs have a tremendous effect on how we analyze and understand the gut microbiome and treat microbiome-mediated diseases. Herein we provide the foundation for using ENBs to determine mediators of the microbiome-gut-brain axis.

### References for Example 2

1. Vuong H E, Yano J M, Fung T C, et al. The Microbiome and Host Behavior. Annu Rev Neurosci. 2017; 40:21-49.
2. Nunes A F, Amaral J D, Lo A C, et al. TUDCA, a bile acid, attenuates amyloid precursor protein processing and amyloid-beta deposition in APP/PS1 mice. Mol Neurobiol. 2012; 45(3):440-54.
3. Shapiro H, Thaiss C A, Levy M, et al. The cross talk between microbiota and the immune system: metabolites take center stage. Curr Opin Immunol. 2014; 30:54-62.
4. McMillin M, Frampton G, Grant S, et al. Bile Acid-Mediated Sphingosine-1-Phosphate Receptor 2 Signaling Promotes Neuroinflammation during Hepatic Encephalopathy in Mice. Front Cell Neurosci. 2017; 11:191.
5. Yanguas-Casas N, Barreda-Manso M A, Nieto-Sampedro M, et al. TUDCA: An Agonist of the Bile Acid Receptor GPBAR1/TGR5 With Anti-Inflammatory Effects in Microglial Cells. J Cell Physiol. 2017; 232(8):2231-45.
6. McMillin M, Frampton G, Quinn M, et al. Bile Acid Signaling Is Involved in the Neurological Decline in a Murine Model of Acute Liver Failure. Am J Pathol. 2016; 186(2):312-23.
7. Huang C, Wang J, Hu W, et al. Identification of functional farnesoid X receptors in brain neurons. FEBS letters. 2016; 590(18):3233-42.
8. Albrecht S, Fleck A K, Kirchberg I, et al. Activation of FXR pathway does not alter glial cell function. J Neuroinflammation. 2017; 14(1):66.
9. Karababa A, Groos-Sahr K, Albrecht U, et al. Ammonia Attenuates LPS-Induced Upregulation of Pro-Inflammatory Cytokine mRNA in Co-Cultured Astrocytes and Microglia. Neurochem Res. 2017; 42(3):737-49.
10. McMillin M, Frampton G, Tobin R, et al. TGR5 signaling reduces neuroinflammation during hepatic encephalopathy. J Neurochem. 2015; 135(3):565-76.
11. Hsiao E Y, McBride S W, Hsien S, et al. Microbiota modulate behavioral and physiological abnormalities associated with neurodevelopmental disorders. Cell. 2013; 155(7):1451-63.
12. Sampson T R, Debelius J W, Thron T, et al. Gut Microbiota Regulate Motor Deficits and Neuroinflammation in a Model of Parkinson's Disease. Cell. 2016; 167(6):1469-80 e12.
13. Krajmalnik-Brown R, Lozupone C, Kang D W, et al. Gut bacteria in children with autism spectrum disorders: challenges and promise of studying how a complex community influences a complex disease. Microb Ecol Health Dis. 2015; 26:26914.
14. Messaoudi M, Lalonde R, Violle N, et al. Assessment of psychotropic-like properties of a probiotic formulation (Lactobacillus helveticus R0052 and Bifidobacterium longum R0175) in rats and human subjects. Br J Nutr. 2011; 105(5):755-64.
15. Erny D, Hrabe de Angelis A L, Jaitin D, et al. Host microbiota constantly control maturation and function of microglia in the CNS. Nature neuroscience. 2015; 18(7):965-77.
16. Wahlstrom A, Sayin S I, Marschall H U, et al. Intestinal Crosstalk between Bile Acids and Microbiota and Its Impact on Host Metabolism. Cell metabolism. 2016; 24(1):41-50.
17. Watanabe M, Houten S M, Mataki C, et al. Bile acids induce energy expenditure by promoting intracellular thyroid hormone activation. Nature. 2006; 439(7075):484-9.
18. Matsubara T, Li F, Gonzalez F J. FXR signaling in the enterohepatic system. Molecular and cellular endocrinology. 2013; 368(1-2):17-29.
19. Pols T W, Noriega L G, Nomura M, et al. The bile acid membrane receptor TGR5 as an emerging target in metabolism and inflammation. Journal of hepatology. 2011; 54(6):1263-72.
20. Joyce S A, MacSharry J, Casey P G, et al. Regulation of host weight gain and lipid metabolism by bacterial bile acid modification in the gut. Proceedings of the National Academy of Sciences of the United States of America. 2014; 111(20):7421-6.
21. Joo S S, Kang H C, Won T J, et al. Ursodeoxycholic acid inhibits pro-inflammatory repertoires, IL-1 beta and nitric oxide in rat microglia. Arch Pharm Res. 2003; 26(12):1067-73.
22. Silva S L, Vaz A R, Diogenes M J, et al. Neuritic growth impairment and cell death by unconjugated bilirubin is mediated by NO and glutamate, modulated by microglia, and prevented by glycoursodeoxycholic acid and interleukin-10. Neuropharmacology. 2012; 62(7):2398-408.
23. Yanguas-Casas N, Barreda-Manso M A, Perez-Rial S, et al. TGFbeta Contributes to the Anti-inflammatory Effects of Tauroursodeoxycholic Acid on an Animal Model of Acute Neuroinflammation. Mol Neurobiol. 2016.
24. Williams K, Alvarez X, Lackner A A. Central nervous system perivascular cells are immunoregulatory cells that connect the CNS with the peripheral immune system. Glia. 2001; 36(2):156-64.
25. Vogt M C, Bruning J C. CNS insulin signaling in the control of energy homeostasis and glucose metabolism—from embryo to old age. Trends in endocrinology and metabolism: TEM. 2013; 24(2):76-84.
26. Gregor M F, Hotamisligil G S. Inflammatory mechanisms in obesity. Annu Rev Immunol. 2011; 29:415-45.
27. Tsaousidou E, Paeger L, Belgardt B F, et al. Distinct Roles for JNK and IKK Activation in Agouti-Related Peptide Neurons in the Development of Obesity and Insulin Resistance. Cell reports. 2014; 9(4):1495-506.
28. Kleinridders A, Schenten D, Konner A C, et al. MyD88 signaling in the CNS is required for development of fatty acid-induced leptin resistance and diet-induced obesity. Cell metabolism. 2009; 10(4):249-59.
29. Guillemot-Legris O, Muccioli G G. Obesity-Induced Neuroinflammation: Beyond the Hypothalamus. Trends Neurosci. 2017; 40(4):237-53.
30. Rivera P, Perez-Martin M, Pavon F J, et al. Pharmacological administration of the isoflavone daidzein enhances cell proliferation and reduces high fat diet-induced apoptosis and gliosis in the rat hippocampus. PloS one. 2013; 8(5):e64750.
31. Jeon B T, Jeong E A, Shin H J, et al. Resveratrol attenuates obesity-associated peripheral and central inflammation and improves memory deficit in mice fed a high-fat diet. Diabetes. 2012; 61(6):1444-54.
32. Kang E B, Koo J H, Jang Y C, et al. Neuroprotective Effects of Endurance Exercise Against High-Fat Diet-Induced Hippocampal Neuroinflammation. J Neuroendocrinol. 2016; 28(5).
33. Krishna S, Keralapurath M M, Lin Z, et al. Neurochemical and electrophysiological deficits in the ventral hippocampus and selective behavioral alterations caused by high-fat diet in female C57BL/6 mice. Neuroscience. 2015; 297:170-81.
34. Camer D, Yu Y, Szabo A, et al. Bardoxolone methyl prevents high-fat diet-induced alterations in prefrontal cortex signalling molecules involved in recognition memory. Prog Neuropsychopharmacol Biol Psychiatry. 2015; 59:68-75.
35. Dutheil S, Ota K T, Wohleb E S, et al. High-Fat Diet Induced Anxiety and Anhedonia: Impact on Brain Homeostasis and Inflammation. Neuropsychopharmacology. 2016; 41(7):1874-87.
36. Pistell P J, Morrison C D, Gupta S, et al. Cognitive impairment following high fat diet consumption is associated with brain inflammation. J Neuroimmunol. 2010; 219(1-2):25-32.
37. Hao S, Dey A, Yu X, et al. Dietary obesity reversibly induces synaptic stripping by microglia and impairs hippocampal plasticity. Brain Behav Immun. 2016; 51:230-9.
38. Lu J, Wu D M, Zheng Y L, et al. Ursolic acid improves high fat diet-induced cognitive impairments by blocking endoplasmic reticulum stress and IkappaB kinase beta/nuclear factor-kappaB-mediated inflammatory pathways in mice. Brain Behav Immun. 2011; 25(8):1658-67.
39. Sivanathan S, Thavartnam K, Arif S, et al. Chronic high fat feeding increases anxiety-like behaviour and reduces transcript abundance of glucocorticoid signalling genes in the hippocampus of female rats. Behav Brain Res. 2015; 286:265-70.
40. Kristensen N B, Bryrup T, Allin K H, et al. Alterations in fecal microbiota composition by probiotic supplementation in healthy adults: a systematic review of randomized controlled trials. Genome medicine. 2016; 8(1):52.
41. Mimee M, Citorik R J, Lu T K. Microbiome therapeutics—Advances and challenges. Adv Drug Deliv Rev. 2016; 105(Pt A):44-54.
42. Walker R I, Owen R L. Intestinal barriers to bacteria and their toxins. Annual review of medicine. 1990; 41:393-400.
43. Zaslaver A, Bren A, Ronen M, et al. A comprehensive library of fluorescent transcriptional reporters for Escherichia coli. Nature methods. 2006; 3(8):623-8.
44. Parks B W, Sallam T, Mehrabian M, et al. Genetic architecture of insulin resistance in the mouse. Cell metabolism. 2015; 21(2):334-46.
45. Crawley J N. Behavioral phenotyping of transgenic and knockout mice: experimental design and evaluation of general health, sensory functions, motor abilities, and specific behavioral tests. Brain research. 1999; 835(1):18-26.
46. Crawley J N. What's wrong with my mouse?: behavioral phenotyping of transgenic and knockout mice. 2nd ed. Hoboken, N.J.: Wiley-Interscience; 2007. xvi, 523 pages p.
47. Winters B D, Forwood S E, Cowell R A, et al. Double dissociation between the effects of peri-postrhinal cortex and hippocampal lesions on tests of object recognition and spatial memory: heterogeneity of function within the temporal lobe. The Journal of neuroscience: the official journal of the Society for Neuroscience. 2004; 24(26):5901-8.
48. Lieben C K, Steinbusch H W, Blokland A. 5,7-DHT lesion of the dorsal raphe nuclei impairs object recognition but not affective behavior and corticosterone response to stressor in the rat. Behav Brain Res. 2006; 168(2):197-207.
49. Mumby D G, Tremblay A, Lecluse V, et al. Hippocampal damage and anterograde object-recognition in rats after long retention intervals. Hippocampus. 2005; 15(8):1050-6.
50. Berlyne D E. Novelty and Curiosity as Determinants of Exploratory Behaviour. B J Psychol-Gen Sect. 1950; 41:68-80.
51. Ennaceur A, Delacour J. A new one-trial test for neurobiological studies of memory in rats. 1: Behavioral data. Behav Brain Res. 1988; 31(1):47-59.
52. Heyser C J, Chemero A. Novel object exploration in mice: not all objects are created equal. Behav Processes. 2012; 89(3):232-8.
53. Njung'e K, Handley S L. Evaluation of marble-burying behavior as a model of anxiety. Pharmacol Biochem Behay. 1991; 38(1):63-7.
54. Thomas A, Burant A, Bui N, et al. Marble burying reflects a repetitive and perseverative behavior more than novelty-induced anxiety. Psychopharmacology (Berl). 2009; 204(2):361-73.
55. Deacon R M. Digging and marble burying in mice: simple methods for in vivo identification of biological impacts. Nat Protoc. 2006; 1(1):122-4.
56. Li X, Risbrough V B, Cates-Gatto C, et al. Comparison of the effects of the GABAB receptor positive modulator BHF177 and the GABAB receptor agonist baclofen on anxiety-like behavior, learning, and memory in mice. Neuropharmacology. 2013; 70:156-67.
57. Bach M E, Hawkins R D, Osman M, et al. Impairment of spatial but not contextual memory in CaMKII mutant mice with a selective loss of hippocampal LTP in the range of the theta frequency. Cell. 1995; 81(6):905-15.
58. Amador-Arjona A, Elliott J, Miller A, et al. Primary cilia regulate proliferation of amplifying progenitors in adult hippocampus: implications for learning and memory. The Journal of neuroscience: the official journal of the Society for Neuroscience. 2011; 31(27):9933-44.
59. Stem L, Chermat R, Thierry B, et al. The automated Tail Suspension Test: a computerized device which differentiates psychotropic drugs. Prog Neuropsychopharmacol Biol Psychiatry. 1987; 11(6):659-71.
60. Sarkisyan G, Roberts A J, Hedlund P B. The 5-HT(7) receptor as a mediator and modulator of antidepressant-like behavior. Behav Brain Res. 2010; 209(1):99-108.
61. Blum-Oehler G, Oswald S, Eiteljorge K, et al. Development of strain-specific PCR reactions for the detection of the probiotic Escherichia coli strain Nissle 1917 in fecal samples. Research in Mirobiology. 2003; 154:59-66.
62. Gosselin D, Link V M, Romanoski C E, et al. Environment drives selection and function of enhancers controlling tissue-specific macrophage identities. Cell. 2014; 159(6):1327-40.

### Example 3

### Colonization of the Human Gut with Re-Introduced and Engineered Commensal Bacteria

Healthy human subjects provide samples of their stool on sterile swabs, which are cultured in the laboratory. Commensal bacterial cells from the stool samples are engineered to express a marker such as a fluorescent protein. The bacteria transformed with a heterologous polynucleotide are subsequently returned to the subject as a concentrated solution of live organisms, e.g., either mixed into a food (e.g., oatmeal, yogurt) or as a suspension in a strongly buffered solution packed into a large gel capsule immediately before consumption. The human subject is provided with sterile swabs for collecting and submitting stool samples at predetermined intervals, e.g., a semiweekly basis for 2 months, to track the success of the engineered bacterial strain in recolonizing in the gastrointestinal tract of the subject.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

SEQUENCE LISTING
SEQ ID NO: 1 - chromosomally integrated polynucleotide sequence for
GFP expression

| | | | |
|---|---|---|---|
| LOCUS | attB_PrplN_Alon | 2832 bp ds-DNA | linear |

13-MAR.-2018

| | |
|---|---|
| COMMENT | attB lambda from 1 to 515 |
| COMMENT | ApEinfo: methylated: 1 |
| FEATURES | Location /Qualifiers |
| promoter | 1408 . . . 1716 |
| | /locus_tag = “Prp1N” |
| | /label = “Prp1N” |
| | /ApEinfo_label = “Prp1N” |
| | /ApEinfo_fwdcolor = “#80ff00” |
| | /ApEinfo_revcolor = “#80ff{00” |
| | /ApEinfo_graphicformat = “arrow_data {{0 1 2 0 0 |
| −1} {} 0}1 | |
| | width 5 offset 0” |
| misc_feature | complement(259 . . . 1283) |
| | /locus_tag = “to_be_replaced” |
| | /label = “to_be_replaced” |
| | /ApEinfo_label = “to_be_replaced” |
| | /ApEinfo_fwdcolor = “#cccccc” |
| | /ApEinfo_revcolor = “#cccccc” |
| | /ApEinfo_graphicformat = “arrow_data {{0 1 2 0 0 |
| −1} {} 0} | |
| | width 5 offset 0” |
| misc_binding | 251 . . . 258 |
| | /locus_tag = “attB_lambda” |
| | /label = “attB_lambda” |
| | /ApEinfo_label = “attB_lambda” |
| | /ApEinfo_fwdcolor = “#ff0080” |
| | /ApEinfo_revcolor = “#ff0080” |
| | /ApEinfo_graphicformat = “arrow_data {{0 1 2 0 0 |
| −1} {} 0} | |
| | width 5 offset 0” |
| CDS | complement(1 . . . 204) |
| | /locus_tag = “ybhC” |
| | /label = “ybhC” |
| | /ApEinfo_label = “ybhC” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| misc_feature | 2832 . . . 2832 |
| | /locus_tag = “towards ter” |
| | /label = “towards_ter” |
| | /ApEinfo_label = “towards_ter” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| misc_feature | 1 . . . 1 |
| | /locus_tag = “towards_ori” |
| | /label = “towards_ori” |
| | /ApEinfo_label = “towards_ori” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| misc_binding | 2576 . . . 2582 |
| | /locus_tag = “attB_lambda(1)” |
| | /label = “attB_lambda(1)” |
| | /ApEinfo_label = “attB_lambda” |
| | /ApEinfo_fwdcolor = “#ff0080” |
| | /ApEinfo_revcolor = “#ff0080” |
| | /ApEinfo_graphicformat = “arrow_data {{0 1 2 0 0 |
| −1} {} 0} | |
| | width 5 offset 0” |
| misc_feature | 1749 . . . 2465 |
| | /locus_tag = “GFPmut2” |
| | /label = “GFPmut2” |
| | /ApEinfo_label = “GFPmut2” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| primer_bind | complement(1929 . . . 1950) |
| | /locus_tag = “qPrimer2_R” |
| | /label = “qPrimer2_R” |
| | /ApEinfo_label = “qPrimer2_R” |
| | /ApEinfo_fwdcolor = “#ff8000” |
| | /ApEinfo_revcolor = “green” |
| | /ApEinfo_graphicformat = “arrow_data {{0 1 2 0 0 |
| −1} {} 0} | |
| | width 5 offset 0” |
| primer_bind | 1848 . . . 1867 |
| | /locus_tag = “qPrimer1” |
| | /label = “qPrimer1” |
| | /ApEinfo_label = “qPrimer1” |
| | /ApEinfo_fwdcolor = “#ff8000” |
| | /ApEinfo_revcolor = “green” |
| | /ApEinfo_graphicformat = “arrow_data {{0 1 2 0 0 |
| −1} {} 0} | |
| | width 5 offset 0” |
| CDS | complement(396 . . . 1187) |
| | /locus_tag = “Kan/neoR” |
| | /label = “Kan/neoR” |
| | /ApEinfo_label = “Kan/neoR” |
| | /ApEinfo_fwdcolor = “yellow” |
| | /ApEinfo_revcolor = “yellow” |
| | /ApEinfo_graphicformat = “arrow_data {{0 1 2 0 0 |
| −1} {} 0} | |
| | width 5 offset 0” |
| misc_feature | 2506 . . . 2545 |
| | /locus_tag = “term?” |
| | /label = “term?” |
| | /ApEinfo_label = “term?” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| misc_feature | 276 . . . 319 |
| | /locus_tag = “term?(1)” |
| | /label = “term?(1)” |
| | /ApEinfo_label = “term?” |
| | /ApEinfo_fwdcolor = “cyan” |
| | /ApEinfo_revcolor = “#00ff00” |
| | /ApEinfo_graphicformat = “arrow_data {{0 1 2 0 0 |
| −1} {} 0} | |
| | width 5 offset 0” |
| primer_bind | 259 . . . 278 |
| | /locus_tag = “P1” |
| | /label = “P1” |
| | /ApEinfo_label = “P1” |
| | /ApEinfo_fwdcolor = “#ff8000” |
| | /ApEinfo_revcolor = “green” |
| | /ApEinfo_graphicformat = “arrow_data {{0 1 2 0 0 |
| −1} {} 0} | |
| | width 5 offset 0” |
| primer_bind | complement(2559 . . . 2575) |
| | /locus_tag = “P2” |
| | /label = “P2” |
| | /ApEinfo_label = “P2” |
| | /ApEinfo_fwdcolor = “#ff8000” |
| | /ApEinfo_revcolor = “green” |
| | /ApEinfo_graphicformat = “arrow_data {{0 1 2 0 0 |
| −1} {} 0} | |
| | width 5 offset 0” |
| misc_feature | complement(1717 . . . 1722) |
| | /locus_tag = “XhoI” |
| | /label = “XhoI” |
| | /ApEinfo_label = “XhoI” |
| | /ApEinfo_fwdcolor = “#ff0000” |
| | /ApEinfo_revcolor = “#ff0000” |
| | /ApEinfo_graphicformat = “arrow_data {{0 1 2 0 0 |
| −1} {} 0} | |
| | width 5 offset 0” |
| misc_feature | complement(1402 . . . 1407) |
| | /locus_tag = “BamHI” |
| | /label = “BamHI” |
| | /ApEinfo_label = “BamHI” |
| | /ApEinfo_fwdcolor = “#ff0000” |
| | /ApEinfo_revcolor = “#ff0000” |
| | /ApEinfo_graphicformat = “arrow_data {{0 1 2 0 0 |
| −1} {} 0} | |
| | width 5 offset 0” |
| CDS | 1635 . . . 1716 |
| | /locus_tag = “rplN” |
| | /label = “rplN” |
| | /ApEinfo_label = “rplN” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| primer_bind | complement(1699 . . . 1716) |
| | /locus_tag = “alon_P1” |
| | /label = “alon_P1” |
| | /ApEinfo_label = “alon_P1” |
| | /ApEinfo_fwdcolor = “#ff8000” |
| | /ApEinfo_revcolor = “green” |
| | /ApEinfo_graphicformat = “arrow_data {{0 1 2 0 0 |
| −1} {} 0} | |
| | width 5 offset 0” |
| primer_bind | 1408 . . . 1425 |
| | /locus_tag = “alon_p2” |
| | /label = “alon_p2” |
| | /ApEinfo_label = “alon_p2” |
| | /ApEinfo_fwdcolor = “#ff8000” |
| | /ApEinfo_revcolor = “green” |
| | /ApEinfo_graphicformat = “arrow_data {{0 1 2 0 0 |
| −1} {} 0} | |
| | width 5 offset 0” |

ORIGIN
CGCTGCAGCA CCTGGTGTCA GGGATGCAAA ATAGTGTTGA GCATCGAAAT TCTGCGCTTC
TTTTGCCGAC AGAATCGGGC GAGAAGAGGT ACCAGGCGCG GTTTGATCAG AAGGACGTTG
ATCGGGCGGG GTTGAGCTAC AGGCGGTCAG CGTCACGCCA AAAGCCAATG CCAGCGCCAG
ACGGGAAACT GAAAATGTGT TCACAGGTTG CTCCGGGCTA TGAAATAGAA AAATGAATCC
GTTGAAGCCT gctTTTTTCC TAGTGCTTGG ATTCTCACCA ATAAAAAACG CCCGGCGGCA
ACCGAGCGTT CTGAACAAAT CCAGATGGAG TTCTGAGGTC ATTACTGGAT CTATCAACAG
GAGTCCAAGC GAGCTCTCGA ACCCCAGAGT CCCGCTCAGA AGAACTCGTC AAGAAGGCGA
TAGAAGGCGA TGCGCTGCGA ATCGGGAGCG GCGATACCGT AAAGCACGAG GAAGCGGTCA
GCCCATTCGC CGCCAAGCTC TTCAGCAATA TCACGGGTAG CCAACGCTAT GTCCTGATAG
CGGTCCGCCA CACCCAGCCG GCCACAGTCG ATGAATCCAG AAAAGCGGCC ATTTTCCACC
ATGATATTCG GCAAGCAGGC ATCGCCATGG GTCACGACGA GATCCTCGCC GTCGGGCATG
CGCGCCTTGA GCCTGGCGAA CAGTTCGGCT GGCGCGAGCC CCTGATGCTC TTCGTCCAGA
TCATCCTGAT CGACAAGACC GGCTTCCATC CGAGTACGTG CTCGCTCGAT GCGATGTTTC
GCTTGGTGGT CGAATGGGCA GGTAGCCGGA TCAAGCGTAT GCAGCCGCCG CATTGCATCA
GCCATGATGG ATACTTTCTC GGCAGGAGCA AGGTGAGATG ACAGGAGATC CTGCCCCGGC
ACTTCGCCCA ATAGCAGCCA GTCCCTTCCC GCTTCAGTGA CAACGTCGAG CACAGCTGCG
CAAGGAACGC CCGTCGTGGC CAGCCACGAT AGCCGCGCTG CCTCGTCCTG CAGTTCATTC
AGGGCACCGG ACAGGTCGGT CTTGACAAAA AGAACCGGGC GCCCCTGCGC TGACAGCCGG
AACACGGCGG CATCAGAGCA GCCGATTGTC TGTTGTGCCC AGTCATAGCC GAATAGCCTC
TCCACCCAAG CGGCCGGAGA ACCTGCGTGC AATCCATCTT GTTCAATCAT GCGAAACGAT
CCTCATCCTG TCTCTTGATC AGATCTTGAT CCCCTGCGCC ATCAGATCCT TGGCGGCAAG
AAAGCCATCC AGTTTACTTT GCAGGGCTTC CCAACCTTAC CAGAGGGCGC CCCAGCTGGC
AATTCCGACG TCTAAGAAAC CATTATTATC ATGACATTAA CCTATAAAAA TAGGCGTATC
ACGAGGCCCT TTCGTCTTCA CGGATCCCGT CCGCTGTCCA AGACTAAATC CTGGACGCTG
GTTCGCGTTG TAGAGAAAGC GGTTCTGTAA TACAGTACAC TCTCTCAATA CGAATAAACG
GCTCAGAAAT GAGCCGTTTA TTTTTTCTAC CCATATCCTT GAAGCGGTGT TATAATGCCG
CGCCCTCGAT ATGGGGATTT TTAACGACCT GATTTTCGGG TCTCAGTAGT AGTTGACATT
AGCGGAGCAC TAAAatgATC CAAGAACAGA CTATGCTGAA CGTCGCCGAC AACTCCGGTG
CACGTCGCGT AATGTGTATC AAGGTTCTGG GTGGCTCTCG AGTCTAGATT TAAGAAGGAG
ATATACATAT GAGTAAAGGA GAAGAACTTT TCACTGGAGT TGTCCCAATT CTTGTTGAAT
TAGATGGTGA TGTTAATGGG CACAAATTTT CTGTCAGTGG AGAGGGTGAA GGTGATGCAA
CATACGGAAA ACTTACCCTT AAATTTATTT GCACTACTGG AAAACTACCT GTTCCATGGC
CAACACTTGT CACTACTTTC GCGTATGGTC TTCAATGCTT TGCGAGATAC CCAGATCATA
TGAAACAGCA TGACTTTTTC AAGAGTGCCA TGCCCGAAGG TTATGTACAG GAAAGAACTA
TATTTTTCAA AGATGACGGG AACTACAAGA CACGTGCTGA AGTCAAGTTT GAAGGTGATA
CCCTTGTTAA TAGAATCGAG TTAAAAGGTA TTGATTTTAA AGAAGATGGA AACATTCTTG
GACACAAATT GGAATACAAC TATAACTCAC ACAATGTATA CATCATGGCA GACAAACAAA
AGAATGGAAT CAAAGTTAAC TTCAAAATTA GACACAACAT TGAAGATGGA AGCGTTCAAC
TAGCAGACCA TTATCAACAA AATACTCCAA TTGGCGATGG CCCTGTCCTT TTACCAGACA
ACCATTACCT GTCCACACAA TCTGCCCTTT CGAAAGATCC CAACGAAAAG AGAGACCACA
TGGTCCTTCT TGAGTTTGTA ACAGCTGCTG GGATTACCCA TGGTATGGAT GAATTGTACA
AATAAATGTC CAGACCTGCA GGCATGCAAG CTCTAGAGGC ATCAAATAAA ACGAAAGGCT
CAGTCGAAAG ACTGGGCCTT TCGTTTTATC TGTTGTTTGT CGGTGAACGC TCTCCATACt
aaCTTGAGCG AAACGGGAAG GTAAAAAGAC AAAAAGTTGT TTTTAATACC TTTAAGTGAT
ACCAGATGGC ATTGCGCCAT CTGGCAGAGT GATTAACTAA ACATCGCAGT AATCGAGGCG
CTTGCCAGAG AGTGGAAATG AACGTTAAAC CCGACCATCG CGCCGCTGGC ACCTTCATCG
ACATCAATAC GTTCTATATC CAGCGCGTGA ACGGTAAAAA TGTAGCGATG AGTTTCGCCT
TTCGGCGGTG CT
SEQ ID NO: 2 - sequence for chromosomal integration site of
heterologous polynucleotides

| | | | |
|---|---|---|---|
| LOCUS | yfgG_pTrc_aph_cc | 4134 bp ds-DNA | linear |

| | |
|---|---|
| 15-FEB.-2017 | |
| DEFINITION | . |
| ACCESSION | |
| VERSION | |
| SOURCE | . |
| ORGANISM | . |
| COMMENT | yfgG_Ptrc-mRuby3_cat from 1 to 3222 |
| COMMENT | yfgG_Ptrc-BSH-FRTcatFRT from 1 to 3483 |
| COMMENT | yfgG_MG1655 from 1 to 694 |
| COMMENT | |
| COMMENT | ApEinfo: methylated: 1 |
| FEATURES | Location/Qualifiers |
| misc_feature | 1 . . . 1 |
| | /label = to_ori |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| promoter | 416 . . . 446 |
| | /label = Ptrc |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| enhancer | 571 . . . 579 |
| | /label = T7g10 enhancer |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| CDS | 641 . . . 659 |
| | /label = mRuby2 |
| | /ApEinfo_fwdcolor = “#b4ffff” |
| | /ApEinfo_revcolor = “#b4ffb2” |
| | /ApEinfo_graphicformat = “arrow_data {{0 1 1 0 0 |
| −1} {} 0} | |
| | width 5 offset 0” |
| CDS | 3295 . . . 3346 |
| | /label = MCS ORF |
| | /ApEinfo_fwdcolor = “#6666ff” |
| | /ApEinfo_revcolor = “#6666ff” |
| | /ApEinfo_graphicformat = “arrow_data {{0 1 2 0 0 |
| −1} {} 0} | |
| | width 5 offset 0” |
| misc_feature | 644 . . . 646 |
| | /label = match_to_msfGFP |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| CDS | 1456 . . . 2247 |
| | /label = Kan/neoR |
| | /ApEinfo_fwdcolor = “yellow” |
| | /ApEinfo_revcolor = “yellow” |
| | /ApEinfo_graphicformat = “arrow_data {{0 1 2 0 0 |
| −1} {} 0} | |
| | width 5 offset 0” |
| misc_feature | 2255 . . . 2263 |
| | /label = overlap |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| promoter | complement(997 . . . 1278) |
| | /label = rhaB3p |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| terminator | 3382 . . . 3539 |
| | /label = rrnB terminator |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| misc_feature | complement(688 . . . 993) |
| | /label = ccdB |
| | /ApEinfo_fwdcolor = “#ff0000” |
| | /ApEinfo_revcolor = “#ff0000” |
| | /ApEinfo_graphicformat = “arrow_data {{0 1 2 0 0 |
| −1} {} 0} | |
| | width 5 offset 0” |
| misc_feature | 4134 . . . 4134 |
| | /label = to_ter |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| primer_bind | complement(2236 . . . 2254) |
| | /label = P1 |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| misc_feature | complement(3277 . . . 3294) |
| | /note = “priming site 1” |
| | /label = priming site 1 |
| | /ApEinfo_fwdcolor = “#00ff80” |
| | /ApEinfo_revcolor = “#00ff80” |
| | /ApEinfo_graphicformat = “arrow_data {{0 1 2 0 0 |
| −1} {} 0} | |
| | width 5 offset 0” |
| misc_feature | 675 . . . 696 |
| | /label = P2 |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| CDS | 2255 . . . 2260 |
| | /label = mRuby2(1) |
| | /ApEinfo_label = “mRuby2” |
| | /ApEinfo_fwdcolor = “#b4ffff” |
| | /ApEinfo_revcolor = “#b4ffb2” |
| | /ApEinfo_graphicformat = “arrow_data {{0 1 1 0 0 |
| −1} {} 0} | |
| | width 5 offset 0” |
| misc_feature | 3243 . . . 3276 |
| | /note = “distal 34-nt of of natural FRT site” |
| | /label = distal 34-nt of of natural FRT site |
| | /ApEinfo_fwdcolor = “1 = “#ff00ff” |
| | /ApEinfo_revcolor = “1 = “#ff00ff” |
| | /ApEinfo_graphicformat = “arrow_data {{0 1 2 0 0 |
| −1} {} 0} | |
| | width 5 offset 0” |
| misc_feature | complement(3063 . . . 3082) |
| | /note = “common priming site c1” |
| | /label = common priming site c1 |
| | /ApEinfo_fwdcolor = “pink” |
| | /ApEinfo_revcolor = “pink” |
| | /ApEinfo_graphicformat = “arrow_data {{0 1 2 0 0 |
| −1} {} 0} | |
| | width 5 offset 0” |
| CDS | 638 . . . 640 |
| | /label = MCS ORF(1) |
| | /ApEinfo_label = “MCS ORF” |
| | /ApEinfo_fwdcolor = “#6666ff” |
| | /ApEinfo_revcolor = “#6666ff” |
| | /ApEinfo_graphicformat = “arrow_data {{0 1 2 0 0 |
| −1} {} 0} | |
| | width 5 offset 0” |
| misc_feature | 2305 . . . 2346 |
| | /note = “distal 42-nt of natural FRT site” |
| | /label = distal 42-nt of natural FRT site |
| | /ApEinfo_fwdcolor = “#ff00ff” |
| | /ApEinfo_revcolor = “#ff00ff” |
| | /ApEinfo_graphicformat = “arrow_data {{0 1 2 0 0 |
| −1} {} 0} | |
| | width 5 offset 0” |
| misc_feature | complement(2264 . . . 2283) |
| | /note = “priming site 2” |
| | /label = priming site 2 |
| | /ApEinfo_fwdcolor = “pink” |
| | /ApEinfo_revcolor = “#ff0080” |
| | /ApEinfo_graphicformat = “arrow_data {{0 1 2 0 0 |
| −1} {} 0} | |
| | width 5 offset 0” |
| misc_feature | 3276 . . . 3294 |
| | /label = New Feature |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| misc_binding | 451 . . . 473 |
| | /label = LacO |
| | /ApEinfo_fwdcolor = “#6495ed” |
| | /ApEinfo_revcolor = “#6495ed” |
| | /ApEinfo_graphicformat = “arrow_data {{0 1 2 0 0 |
| −1} {} 0} | |
| | width 5 offset 0” |
| misc_feature | 3235 . . . 3242 |
| | /label = more FRT |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| misc_feature | 3928 . . . 3946 |
| | /label = SDB015 |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| misc_feature | 489 . . . 558 |
| | /label = rrnB antiterminator |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| RBS | 595 . . . 600 |
| | /label = rbs |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| terminator | 3347 . . . 3736 |
| | /label = rrnB Term Region DNA |
| | /ApEinfo_fwdcolor = “#18c3c0” |
| | /ApEinfo_revcolor = “#1ac703” |
| | /ApEinfo_graphicformat = “arrow_data {{0 1 2 0 0 |
| −1} {} 0} | |
| | width 5 offset 0” |
| CDS | 660 . . . 674 |
| | /label = mRuby3 |
| | /ApEinfo_fwdcolor = “#ff0000” |
| | /ApEinfo_revcolor = “green” |
| | /ApEinfo_graphicformat = “arrow_data {{0 1 2 0 0 |
| −1} {} 0} | |
| | width 5 offset 0” |
| misc_feature | 139 . . . 163 |
| | /label = SDB016 |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| misc_feature | complement(3276 . . . 3294) |
| | /note = “priming site 3” |
| | /label = priming site 3 |
| | /ApEinfo_fwdcolor = “pink” |
| | /ApEinfo_revcolor = “pink” |
| | /ApEinfo_graphicformat = “arrow_data {{0 1 2 0 0 |
| −1} {} 0} | |
| | width 5 offset 0” |
| CDS | 2555 . . . 3193 |
| | /note = “Tn9” |
| | /codon_start = 1 |
| | /transl_table = 11 |
| | /product = “chloramphenicol acetyl transferase” |
| | /protein_id = “AAL02033.1” |
| | /db_xref = “GI: 15554330” |

/translation = “MEKKITGYTTVDISQWHRKEHFEAFQSVAQCTYNQTVQLDITAF
LKTVKKNKHKFYPAFIHILARLMNAHPELRMAMKDGELVIWDSVHPCYTVFHEQTETF
SSLWSEYHDDFRQFLHIYSQDVACYGENLAYFPKGFIENMFFVSANPWVSFTSFDLNV
ANMDNFFAPVFTMGKYYTQGDKVLMPLAIQVHHAVCDGFHVGRCLMNTTVLR”

| | |
|---|---|
| | /label = chloramphenicol acetyl transferase |
| | /ApEinfo_fwdcolor = “pink” |
| | /ApEinfo_revcolor = “pink” |
| | /ApEinfo_graphicformat = “arrow_data {{0 1 2 0 0 |
| −1} {} 0} | |
| | width 5 offset 0” |
| misc_feature | complement(2353 . . . 2372) |
| | /note = “common priming site c2” |
| | /label = common priming site c2 |
| | /ApEinfo_fwdcolor = “pink” |
| | /ApEinfo_revcolor = “pink” |
| | /ApEinfo_graphicformat = “arrow_data {{0 1 2 0 0 |
| −1} {} 0} | |
| | width 5 offset 0” |
| rep_origin | complement(2264 . . . 2280) |
| | /note = “R6K gamma replication origin” |
| | /label = R6K gamma replication origin |
| | /ApEinfo_fwdcolor = “pink” |
| | /ApEinfo_revcolor = “pink” |
| | /ApEinfo_graphicformat = “arrow_data {{0 1 2 0 0 |
| −1} {} 0} | |
| | width 5 offset 0” |
| primer_bind | complement(3277 . . . 3294) |
| | /label = SJO752 |
| | /ApEinfo_fwdcolor = “#0d6d6c” |
| | /ApEinfo_revcolor = “green” |
| | /ApEinfo_graphicformat = “arrow_data {{0 1 2 0 0 |
| −1} {} 0} | |
| | width 5 offset 0” |
| misc_feature | 3690 . . . 3717 |
| | /label = rrnBT2 |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| enhancer | 608 . . . 634 |
| | /label = Minicistron |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| promoter | 349 . . . 395 |
| | /label = cryptic promoter?+0 |
| | /ApEinfo_fwdcolor = “#66ffff” |
| | /ApEinfo_revcolor = “#0080ff” |
| | /ApEinfo_graphicformat = “arrow_data {{0 1 2 0 0 |
| −1} {} 0} | |
| | width 5 offset 0” |
| primer_bind | 641 . . . 659 |
| | /label = hupA-mR2_F1 |
| | /ApEinfo_fwdcolor = “#1297ff” |
| | /ApEinfo_revcolor = “#26e52e” |
| | /ApEinfo_graphicformat = “arrow_data {{0 1 1 0 0 |
| −1} {} 0} | |
| | width 5 offset 0” |
| primer_bind | 349 . . . 367 |
| | /label = SDB001_priming |
| | /ApEinfo_fwdcolor = “#ff8000” |
| | /ApEinfo_revcolor = “green” |
| | /ApEinfo_graphicformat = “arrow_data {{0 1 2 0 0 |
| −1} {} 0} | |
| | width 5 offset 0” |
| primer_bind | complement(3772 . . . 3790) |
| | /label = SDB002 |
| | /ApEinfo_fwdcolor = “#ff8000” |
| | /ApEinfo_revcolor = “green” |
| | /ApEinfo_graphicformat = “arrow_data {{0 1 2 0 0 |
| −1} {} 0} | |
| | width 5 offset 0” |
| primer_bind | complement(2255 . . . 2263) |
| | /label = hupA-mR2_R1 |
| | /ApEinfo_fwdcolor = “#1297ff” |
| | /ApEinfo_revcolor = “#26e52e” |
| | /ApEinfo_graphicformat = “arrow_data {{0 1 1 0 0 |
| −1} {} 0} | |
| | width 5 offset 0” |
| primer_bind | 308 . . . 367 |
| | /label = SDB013 |
| | /ApEinfo_fwdcolor = “#cccccc” |
| | /ApEinfo_revcolor = “#cccccc” |
| | /ApEinfo_graphicformat = “arrow_data {{0 1 2 0 0 |
| -1} {} 0} | |
| | width 5 offset 0” |
| misc_feature | complement(3772 . . . 3831) |
| | /label = SDB014 |
| | /ApEinfo_fwdcolor = “#cccccc” |
| | /ApEinfo_revcolor = “#cccccc” |
| | /ApEinfo_graphicformat = “arrow_data {{0 1 2 0 0 |
| −1} {} 0} | |
| | width 5 offset 0” |

ORIGIN
AAATGTATTG CGTATAAAGG GATAGATTAA ATTTATTAAA ATTCACGCAG GGAATAATTT
ATAAAAATAT ATCATTACAA CATATATATT AATTTTATAT TTATTTCGCA GCCAAACAGG
ATAAACATCA GTCAGGATAA AGACGTAAAA TTTAAGATGA AAGAATATCT GCCGCTTTGA
TAATCCCAAA CAGCAAAAGC ATCCGACAGC GAAGTGCCGT TATGTTCTGT GAAAGTGGAG
AAGGTAACCG ctaACGTTGT ACCGGTGATT CGACGGAGAG TGTGGATTGC TGAGCTTCTT
TTTTGCTCTG ATGGTGCTGC CAGGCACCGA CGGACGAGTA GATAAAACCT CCCGTTCTGG
ATAATGTTTT TTGCGCCGAC ATCATAACGG TTCTGGCAAA TATTCTGAAA TGAGCTGTTG
ACAATTAATC ATCCGGCTCG TATAATGTGT GGAATTGTGA GCGGATAACA ATTTCACACA
GGAAACAGCG CCGCTGAGAA AAAGCGAAGC GGCACTGCTC TTTAACAATT TATCAGACAA
TCTGTGTGGG CACTCGACCG GAATTATCGA TTAACTTTAT TATTAAAAAT TAAAGAGGTA
TATATTAATG TATCGATTAA ATAAGGAGGA ATAAACCATG GTGTCTAaGG GCGAAGAGcT
GATCAAGGAA AATAcggagc ctgacattta tattccccag aacatcaggt taatggcgtt
tttgatgtca ttttcgcggt ggctgagatc agccacttct tccccgataa cggagaccgg
cacaCtggcc atatcggtgg tcatcatgcg ccagctttca tccccgatat gcaccaccgg
gtaaagttca cgggagactt tatctgacag cagacgtgca ctggccaggg ggatcaccat
ccgtcgcccg ggcgtgtcaa taatatcact ctgtacatcc acaaacagac gataacggct
ctctctttta taggtgtaaa ccttaaactg catatggtga tcctgctgaa tttcattacg
accagtctaa aaagcgcctg aattcgcgac cttctcgtta ctgacaggaa aatgggccat
tggcaaccag ggaaagatga acgtgatgat gttcacaatt tgctgaattg tggtgatgtg
atgctcaccg catttcctga aaattcacgc tgtatcttga aaaatcgacg ttttttacgt
ggttttccgt cgaaaattta aggtaagaac ctgacctcgt gattactatt tcgccgtgtt
gacgacatca ggaggccacc atggcctcga ctagttccgg agacagcaag cgaaccggaa
ttgccagctg gggcgccctc tggtaaggtt gggaagccct gcaaagtaaa ctggatggct
ttcttgccgc caaggatctg atggcgcagg ggatcaagat ctgatcaaga gacaggatga
ggatcgtttc gcatgattga acaagatgga ttgcacgcag gttctccggc cgcttgggtg
gagaggctat tcggctatga ctgggcacaa cagacaatcg gctgctctga tgccgccgtg
ttccggctgt cagcgcaggg gcgcccggtt ctttttgtca agaccgacct gtccggtgcc
ctgaatgaac tgcaggacga ggcagcgcgg ctatcgtggc tggccacgac gggcgttcct
tgcgcagctg tgctcgacgt tgtcactgaa gcgggaaggg actggctgct attgggcgaa
gtgccggggc aggatctcct gtcatctcac cttgctcctg ccgagaaagt atccatcatg
gctgatgcaa tgcggcggct gcatacgctt gatccggcta cctgcccatt cgaccaccaa
gcgaaacatc gcatcgagcg agcacgtact cggatggaag ccggtcttgt cgatcaggat
gatctggacg aagagcatca ggggctcgcg ccagccgaac tgttcgccag gctcaaggcg
cgcatgcccg acggcgagga tctcgtcgtg acccatggcg atgcctgctt gccgaatatc
atggtggaaa atggccgctt ttctggattc atcgactgtg gccggctggg tgtggcggac
cgctatcagg acatagcgtt ggctacccgt gatattgctg aagagcttgg cggcgaatgg
gctgaccgct tcctcgtgct ttacggtatc gccgctcccg attcgcagcg catcgccttc
tatcgccttc ttgacgagtt cttctgagcg ggactacaag TAAatgggaa ttagccatgg
tccatatgaa tatcctcctt agttcctatt ccgaagttcc tattctctag aaagtatagg
aacttcggcg cgcctacctg tgacggaaga tcacttcgca gaataaataa atcctggtgt
ccctgttgat accgggaagc cctgggccaa cttttggcga aaatgagacg ttgatcggca
cgtaagaggt tccaactttc accataatga aataagatca ctaccgggcg tattttttga
gttgtcgaga ttttcaggag ctaaggaagc taaaatggag aaaaaaatca ctggatatac
caccgttgat atatcccaat ggcatcgtaa agaacatttt gaggcatttc agtcagttgc
tcaatgtacc tataaccaga ccgttcagct ggatattacg gcctttttaa agaccgtaaa
gaaaaataag cacaagtttt atccggcctt tattcacatt cttgcccgcc tgatgaatgc
tcatccggaa ttacgtatgg caatgaaaga cggtgagctg gtgatatggg atagtgttca
cccttgttac accgttttcc atgagcaaac tgaaacgttt tcatcgctct ggagtgaata
ccacgacgat ttccggcagt ttctacacat atattcgcaa gatgtggcgt gttacggtga
aaacctggcc tatttcccta aagggtttat tgagaatatg tttttcgtct cagccaatcc
ctgggtgagt ttcaccagtt ttgatttaaa cgtggccaat atggacaact tcttcgcccc
cgttttcacc atgggcaaat attatacgca aggcgacaag gtgctgatgc cgctggcgat
tcaggttcat catgccgttt gtgatggctt ccatgtcggc agatgcttaa tgaatacaac
agtactgcga tgagtggcag ggcggggcgt aaggcgcgcc atttaaatga agttcctatt
ccgaagttcc tattctctag aaagtatagg aacttcgaag cagctccagc ctacGGGAAT
TCGAAGCTTG GCTGTTTTGG CGGATGAGAG AAGATTTTCA GCCTGATACA GATTAAATCA
GAACGCAGAA GCGGTCTGAT AAAACAGAAT TTGCCTGGCG GCAGTAGCGC GGTGGTCCCA
CCTGACCCCA TGCCGAACTC AGAAGTGAAA CGCCGTAGCG CCGATGGTAG TGTGGGGTCT
CCCCATGCGA GAGTAGGGAA CTGCCAGGCA TCAAATAAAA CGAAAGGCTC AGTCGAAAGA
CTGGGCCTTT CGTTTTATCT GTTGTTTGTC GGTGAACGCT CTCCTGAGTA GGACAAATCC
GCCGGGAGCG GATTTGAACG TTGCGAAGCA ACGGCCCGGA GGGTGGCGGG CAGGACGCCC
GCCATAAACT GCCAGGCATC AAATTAAGCA GAAGGCCATC CTGACGGATG GCCTTTTTGC
GTTTCTACAA ACTCTTTTTG TTTATTTTTC TAAATACATT CAAATATGTA TCCGCTCATG
AGACAATAAC GGCCAAAGAA GAAGATAAAG CTGATGAGCA GTACGATACG GGTCATGCGA
CTGTTAAATC GGTGTCGTTT TCGCATACTG GTTGCCTGAC TcacAAAAGG TTCCTTGAAG
TATGTCCCAC GCTGTGGACG GTACTTACAT TAAGGCACAA CAGGACAAAA TGGTCAATTC
TTCGTTATGT AAAAAAGCGT CAGTGGATAC ATATTTTAAT GTTATGGAAG TTAATTTAAT
TATTTACAAT GATGATGTAA TAATGATGAA TTAACATGAT AAACGTAATA ATTCATTAAT
CAGGGTTATT TTGTTTGATA TATATCAATT GGTACTTTCA TATGACACTT AGAA
SEQ ID NO: 3 -sequence in the chromosome which contains and drives
expression of BSH (which can be replaced with another heterologous
polynucleotide, e.g., SULT2A1, 7alphaHSDH, IL-10, Amuc_1100)

| | | | |
|---|---|---|---|
| LOCUS | yfgG_Ptrc_BSB_FR | 3483 bp ds-DNA | linear |

| | |
|---|---|
| 19-JAN.-2018 | |
| DEFINITION | . |
| ACCESSION | |
| VERSION | |
| SOURCE | . |
| ORGANISM | . |
| COMMENT | yfgG_MG1655 from 1 to 694 |
| COMMENT | ApEinfo: methylated: 1 |
| FEATURES | Location/Qualifiers |
| misc_feature | 1 . . . 1 |
| | /locus_tag = “to_ori” |
| | /label = “to_ori” |
| | /ApEinfo_label = “to_ori” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| promoter | 416 . . . 446 |
| | /locus_tag = “Ptrc” |
| | /label = “Ptrc” |
| | /ApEinfo_label = “Ptrc” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| enhancer | 571 . . . 579 |
| | /locus_tag = “T7g10 enhancer” |
| | /label = “T7g10 enhancer” |
| | /ApEinfo_label = “T7g10 enhancer” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| CDS | 2644 . . . 2695 |
| | /locus_tag = “MCS ORF” |
| | /label = “MCS ORF” |
| | /ApEinfo_label = “MCS ORF” |
| | /ApEinfo_fwdcolor = “#6666ff” |
| | /ApEinfo_revcolor = “#6666ff” |
| | /ApEinfo_graphicformat = “arrow_data {{0 1 2 0 0 |
| −1} {} 0} | |
| | width 5 offset 0” |
| terminator | 2731 . . . 2888 |
| | /locus_tag = “rrnB terminator” |
| | /label = “rrnB terminator” |
| | /ApEinfo_label = “rrnB terminator” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| misc_feature | 3483 . . . 3483 |
| | /locus_tag = “to_ter” |
| | /label = “to_ter” |
| | /ApEinfo_label = “to_ter” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| misc_feature | complement(2626 . . . 2643) |
| | /note = “priming site 1” |
| | /locus_tag = “priming site 1” |
| | /label = “priming site 1” |
| | /ApEinfo_label = “priming site 1” |
| | /ApEinfo_fwdcolor = “#00ff80” |
| | /ApEinfo_revcolor = “#00ff80” |
| | /ApEinfo_graphicformat = “arrow_data {{0 1 2 0 0 |
| −1} {} 0} | |
| | width 5 offset 0” |
| misc_feature | 2592 . . . 2625 |
| | /note = “distal 34-nt of of natural FRT site” |
| | /locus_tag = “distal 34-nt of of natural FRT |
| site” | |
| | /label = “distal 34-nt of of natural FRT site” |
| | /ApEinfo_label = “distal 34-nt of of natural FRT |
| site” | |
| | /ApEinfo_fwdcolor = “#ff00ff” |
| | /ApEinfo_revcolor = “#ff00ff” |
| | /ApEinfo_graphicformat = “arrow_data {{0 1 2 0 0 |
| −1} {} 0} | |
| | width 5 offset 0” |
| misc_feature | complement(2412 . . . 2431) |
| | /note = “common priming site c1” |
| | /locus_tag = “common priming site c1” |
| | /label = “common priming site c1” |
| | /ApEinfo_label = “common priming site c1” |
| | /ApEinfo_fwdcolor = “pink” |
| | /ApEinfo_revcolor = “pink” |
| | /ApEinfo_graphicformat = “arrow_data {{0 1 2 0 0 |
| −1} {} 0} | |
| | width 5 offset 0” |
| misc_feature | 1654 . . . 1695 |
| | /note = “distal 42-nt of natural FRT site” |
| | /locus_tag = “distal 42-nt of natural FRT site” |
| | /label = “distal 42-nt of natural FRT site” |
| | /ApEinfo_label = “distal 42-nt of natural FRT |
| site” | |
| | /ApEinfo_fwdcolor = “#ff00ff” |
| | /ApEinfo_revcolor = “#ff00ff” |
| | /ApEinfo_graphicformat = “arrow_data {{0 1 2 0 0 |
| −1} {} 0} | |
| | width 5 offset 0” |
| misc_feature | complement(1613 . . . 1632) |
| | /note = “priming site 2” |
| | /locus_tag = “priming site 2” |
| | /label = “priming site 2” |
| | /ApEinfo_label = “priming site 2” |
| | /ApEinfo_fwdcolor = “pink” |
| | /ApEinfo_revcolor = “#{{0080” |
| | /ApEinfo_graphicformat = “arrow_data {{0 1 2 0 0 |
| −1} {} 0} | |
| | width 5 offset 0” |
| misc_feature | 2625 . . . 2643 |
| | /locus_tag = “New Feature” |
| | /label = “New Feature” |
| | /ApEinfo_label = “New Feature” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| misc_binding | 451 . . . 473 |
| | /locus_tag = “LacO” |
| | /label = “LacO” |
| | /ApEinfo_label = “LacO” |
| | /ApEinfo_fwdcolor = “#6495ed” |
| | /ApEinfo_revcolor = “#6495ed” |
| | /ApEinfo_graphicformat = “arrow_data {{0 1 2 0 0 |
| −1} {} 0} | |
| | width 5 offset 0” |
| misc_feature | 2584 . . . 2591 |
| | /locus_tag = “more FRT” |
| | /label = “more FRT” |
| | /ApEinfo_label = “more FRT” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| misc_feature | 3277 . . . 3295 |
| | /locus_tag = “SDB015” |
| | /label = “SDB015” |
| | /ApEinfo_label = “SDB015” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| misc_feature | 489 . . . 558 |
| | /locus_tag = “rrnB antiterminator” |
| | /label = “rrnB antiterminator” |
| | /ApEinfo_label = “rrnB antiterminator” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| RBS | 595 . . . 600 |
| | /locus_tag = “rbs” |
| | /label = “rbs” |
| | /ApEinfo_label = “rbs” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| terminator | 2696 . . . 3085 |
| | /locus_tag = “rrnB Term Region DNA” |
| | /label = “rrnB Term Region DNA” |
| | /ApEinfo_label = “rrnB Term Region DNA” |
| | /ApEinfo_fwdcolor = “#18c3c0” |
| | /ApEinfo_revcolor = “#lac703” |
| | /ApEinfo_graphicformat = “arrow_data {{0 1 2 0 0 |
| −1} {} 0} | |
| | width 5 offset 0” |
| misc_feature | 139 . . . 163 |
| | /locus_tag = “SDB016” |
| | /label = “SDB016” |
| | /ApEinfo_label = “SDB016” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| misc_feature | complement(2625 . . . 2643) |
| | /note = “priming site 3” |
| | /locus_tag = “priming site 3” |
| | /label = “priming site 3” |
| | /ApEinfo_label = “priming site 3” |
| | /ApEinfo_fwdcolor = “pink” |
| | /ApEinfo_revcolor = “pink” |
| | /ApEinfo_graphicformat = “arrow_data {{0 1 2 0 0 |
| −1} {} 0} | |
| | width 5 offset 0” |
| CDS | 1904 . . . 2542 |
| | /note = “Tn9” |
| | /codon_start = 1 |
| | /transl_table = 11 |
| | /product = “chloramphenicol acetyl transferase” |
| | /protein_id = “AAL02033.1” |
| | /db_xref = “GI: 15554330” |

/translation = “MEKKITGYTTVDISQWHRKEHFEAFQSVAQCTYNQTVQLDITAF
LKTVKKNKHKFYPAFIHILARLMNAHPELRMAMKDGELVTWDSVHPCYTVFHEQTETF
SSLWSEYHDDFRQFLHIYSQDVACYGENLAYFPKGFIENMFFVSANWVSFTSFDLNV
ANMDNFFAPVFTMGKYYTQGDKVLMPLAIQVHHAVCDGFHVGRCLMNTTVLR”

| | |
|---|---|
| | /locus_tag = “chloramphenicol acetyl |
| transferase” | |
| | /label = “chloramphenicol acetyl transferase” |
| | /ApEinfo_label = “chloramphenicol acetyl |
| transferase” | |
| | /ApEinfo_fwdcolor = “pink” |
| | /ApEinfo_revcolor = “pink” |
| | /ApEinfo_graphicformat = “arrow_data {{0 1 2 0 0 |
| −1} {} 0} | |
| | width 5 offset 0” |
| misc_feature | complement(1702 . . . 1721) |
| | /note = “common priming site c2” |
| | /locus_tag = “common priming site c2” |
| | /label = “common priming site c2” |
| | /ApEinfo_label = “common priming site c2” |
| | /ApEinfo_fwdcolor = “pink” |
| | /ApEinfo_revcolor = “pink” |
| | /ApEinfo_graphicformat = “arrow_data {{0 1 2 0 0 |
| −1} {} 0} | |
| | width 5 offset 0” |
| rep_origin | complement(1613 . . . 1629) |
| | /note = “R6K gamma replication origin” |
| | /locus_tag = “R6K gamma replication origin” |
| | /label = “R6K gamma replication origin” |
| | /ApEinfo_label = “R6K gamma replication origin” |
| | /ApEinfo_fwdcolor = “pink” |
| | /ApEinfo_revcolor = “pink” |
| | /ApEinfo_graphicformat = “arrow_data {{0 1 2 0 0 |
| −1} {} 0} | |
| | width 5 offset 0” |
| primer_bind | complement(2626 . . . 2643) |
| | /locus_tag = “SJO752” |
| | /label = “SJO752” |
| | /ApEinfo_label = “SJO752” |
| | /ApEinfo_fwdcolor = “#0d6d6c” |
| | /ApEinfo_revcolor = “green” |
| | /ApEinfo_graphicformat = “arrow_data {{0 1 2 0 0 |
| −1} {} 0} | |
| | width 5 offset 0” |
| misc_feature | 3039 . . . 3066 |
| | /locus_tag = “rrnBT2” |
| | /label = “rrnBT2” |
| | /ApEinfo_label = “rrnBT2” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| CDS | 638 . . . 1612 |
| | /locus_tag = “BSH1” |
| | /label = “BSH1” |
| | /ApEinfo_label = “BSH1” |
| | /ApEinfo_fwdcolor = “cyan” |
| | /ApEinfo_revcolor = “green” |
| | /ApEinfo_graphicformat = “arrow_data {{0 1 2 0 0 |
| −1} {} 0} | |
| | width 5 offset 0” |
| primer_bind | complement(1105 . . . 1125) |
| | /locus_tag = “qPrimer2” |
| | /label = “qPrimer2” |
| | /ApEinfo_label = “qPrimer2” |
| | /ApEinfo_fwdcolor = “#ff8000” |
| | /ApEinfo_revcolor = “green” |
| | /ApEinfo_graphicformat = “arrow_data {{0 1 2 0 0 |
| −1} {} 0} | |
| | width 5 offset 0” |
| primer_bind | 992 . . . 1015 |
| | /locus_tag = “qPrimer1” |
| | /label = “qPrimer1” |
| | /ApEinfo_label = “qPrimer1” |
| | /ApEinfo_fwdcolor = “#ff8000” |
| | /ApEinfo_revcolor = “green” |
| | /ApEinfo_graphicformat = “arrow_data {{0 1 2 0 0 |
| −1} {} 0} | |
| | width 5 offset 0” |
| enhancer | 608 . . . 634 |
| | /locus_tag = “Minicistron” |
| | /label = “Minicistron” |
| | /ApEinfo_label = “Minicistron” |
| | /ApEinfo_fwdcolor = “cyan” |
| | /ApEinfo_revcolor = “green” |
| | /ApEinfo_graphicformat = “arrow_data {{0HO 1 2 0 0 |
| −1} {} 0} | |
| | width 5 offset 0” |
| promoter | 349 . . . 395 |
| | /locus_tag = “cryptic promoter?” |
| | /label = “cryptic promoter?” |
| | /ApEinfo_label = “cryptic promoter?” |
| | /ApEinfo_fwdcolor = “#66ffff” |
| | /ApEinfo_revcolor = “#0080ff” |
| | /ApEinfo_graphicformat = “arrow_data {{0 1 2 0 0 |
| −1} {} 0} | |
| | width 5 offset 0” |
| primer_bind | 349 . . . 367 |
| | /locus_tag = “SDB001_priming” |
| | /label = “SDB001_priming” |
| | /ApEinfo_label = “SDB001_priming” |
| | /ApEinfo_fwdcolor = “#ff8000” |
| | /ApEinfo_revcolor = “green” |
| | /ApEinfo_graphicformat = “arrow_data {{0 1 2 0 0 |
| −1} {} 0} | |
| | width 5 offset 0” |
| primer_bind | complement(3121 . . . 3139) |
| | /locus_tag = “SDB002” |
| | /label = “SDB002” |
| | /ApEinfo_label = “SDB002” |
| | /ApEinfo_fwdcolor = “#ff8000” |
| | /ApEinfo_revcolor = “green” |
| | /ApEinfo_graphicformat = “arrow_data {{0 1 2 0 0 |
| −1} {} 0} | |
| | width 5 offset 0” |
| primer_bind | 550 . . . 569 |
| | /locus_tag = “SDB027” |
| | /label = “SDB027” |
| | /ApEinfo_label = “SDB027” |
| | /ApEinfo_fwdcolor = “#ff8000” |
| | /ApEinfo_revcolor = “green” |
| | /ApEinfo_graphicformat = “arrow_data {{0 1 2 0 0 |
| −1} {} 0} | |
| | width 5 offset 0” |

ORIGIN
AAATGTATTG CGTATAAAGG GATAGATTAA ATTTATTAAA ATTCACGCAG GGAATAATTT
ATAAAAATAT ATCATTACAA CATATATATT AATTTTATAT TTATTTCGCA GCCAAACAGG
ATAAACATCA GTCAGGATAA AGACGTAAAA TTTAAGATGA AAGAATATCT GCCGCTTTGA
TAATCCCAAA CAGCAAAAGC ATCCGACAGC GAAGTGCCGT TATGTTCTGT GAAAGTGGAG
AAGGTAACCG ctaACGTTGT ACCGGTGATT CGACGGAGAG TGTGGATTGC TGAGCTTCTT
TTTTGCTCTG ATGGTGCTGC CAGGCACCGA CGGACGAGTA GATAAAACCT CCCGTTCTGG
ATAATGTTTT TTGCGCCGAC ATCATAACGG TTCTGGCAAA TATTCTGAAA TGAGCTGTTG
ACAATTAATC ATCCGGCTCG TATAATGTGT GGAATTGTGA GCGGATAACA ATTTCACACA
GGAAACAGCG CCGCTGAGAA AAAGCGAAGC GGCACTGCTC TTTAACAATT TATCAGACAA
TCTGTGTGGG CACTCGACCG GAATTATCGA TTAACTTTAT TATTAAAAAT TAAAGAGGTA
TATATTAATG TATCGATTAA ATAAGGAGGA ATAAACCatg TGCACCGCGA TeACCCTGAA
TGGcAAcTCT AACTACTTTG GTCGTAACCT GGATCTGGAC TTCTCTTACG GTGAGCAGGT
GATCATTACC CCGGCTGAGT ACGAATTTAA GTTCCGTAAA GAAAAAGCGA TCAAAAACCA
CAAATCCCTg ATCGGTGTAG GTATCGTTGC GAACGCGTAT CCGCTgTACT TCGACGCAAT
CAATGAAGAT GGTCTGGGTA TGGCCGGTCT gAACTTCCCG GGTAACGCAT ACTACTCCGA
CGCGCTGGAA AACGACAAAG ACAACATCAC CCCGTTCGAA TTCATCCCGT GGATCCTGCG
TCAATGTAGC GACGTAAACG AAGCGCGTAA TCTgGTCGAA CGTATTAACC TgATCAACCT
gTCCTTTTCT GAACAGCTGC CTCTGGCGGG TCTGCACTGG CTGATCGCGG ACCGTGAAAA
GTCTATTGTT GTTGAAGTTA CCAAGTCTGG TGTTCACATC TACGACAACC CTATTGGCGT
TCTgACCAAC AACCCGGAAT TCAACTACCA GATGTACAAC CTGAACAAAT ACCGCAATCT
GTCTATCTCT ACCCCGCAGA ACACCTTCTC TGACTCTGTT GACCTGAAAG TAGACGGCAC
CGGTTTCGGT GGCATCGGTC TGCCTGGTGA CGTTTCTCCG GAAAGCCGTT TCGTTCGTGC
TGCTTTCTCT AAACTGAACT CTTCTAAAGG TACGACGGTT GAAGAAGATA TTACCCAGTT
CTTCCACATC CTGGGTACCG TTGAACAGAT CAAGGGTGTT AATAAGACCG AATCTGGCAA
AGAAGAATAC ACCGTTTACA GCAACTGCTA CGACCTgGAC AACAAGACGC TGTACTACAC
CACCTACGAA AACCGTCAGA TTGTTGCCGT AACCCTgAAC GAAGACAAAA ACGGTAACGG
CCTgATTGCG TACCCTTTTG AACGCAAACA GGTTATCAAC AAACTgAACt aaatgggaat
tagccatggt ccatatgaat atcctcctta gttcctattc cgaagttcct attctctaga
aagtatagga acttcggcgc gcctacctgt gacggaagat cacttcgcag aataaataaa
tcctggtgtc cctgttgata ccgggaagcc ctgggccaac ttttggcgaa aatgagacgt
tgatcggcac gtaagaggtt ccaactttca ccataatgaa ataagatcac taccgggcgt
attttttgag ttgtcgagat tttcaggagc taaggaagct aaaatggaga aaaaaatcac
tggatatacc accgttgata tatcccaatg gcatcgtaaa gaacattttg aggcatttca
gtcagttgct caatgtacct ataaccagac cgttcagctg gatattacgg cctttttaaa
gaccgtaaag aaaaataagc acaagtttta tccggccttt attcacattc ttgcccgcct
gatgaatgct catccggaat tacgtatggc aatgaaagac ggtgagctgg tgatatggga
tagtgttcac ccttgttaca ccgttttcca tgagcaaact gaaacgtttt catcgctctg
gagtgaatac cacgacgatt tccggcagtt tctacacata tattcgcaag atgtggcgtg
ttacggtgaa aacctggcct atttccctaa agggtttatt gagaatatgt ttttcgtctc
agccaatccc tgggtgagtt tcaccagttt tgatttaaac gtggccaata tggacaactt
cttcgccccc gttttcacca tgggcaaata ttatacgcaa ggcgacaagg tgctgatgcc
gctggcgatt caggttcatc atgccgtttg tgatggcttc catgtcggca gatgcttaat
gaatacaaca gtactgcgat gagtggcagg gcggggcgta aggcgcgcca tttaaatgaa
gttcctattc cgaagttcct attctctaga aagtatagga acttcgaagc agctccagcc
tacGGGAATT CGAAGCTTGG CTGTTTTGGC GGATGAGAGA AGATTTTCAG CCTGATACAG
ATTAAATCAG AACGCAGAAG CGGTCTGATA AAACAGAATT TGCCTGGCGG CAGTAGCGCG
GTGGTCCCAC CTGACCCCAT GCCGAACTCA GAAGTGAAAC GCCGTAGCGC CGATGGTAGT
GTGGGGTCTC CCCATGCGAG AGTAGGGAAC TGCCAGGCAT CAAATAAAAC GAAAGGCTCA
GTCGAAAGAC TGGGCCTTTC GTTTTATCTG TTGTTTGTCG GTGAACGCTC TCCTGAGTAG
GACAAATCCG CCGGGAGCGG ATTTGAACGT TGCGAAGCAA CGGCCCGGAG GGTGGCGGGC
AGGACGCCCG CCATAAACTG CCAGGCATCA AATTAAGCAG AAGGCCATCC TGACGGATGG
CCTTTTTGCG TTTCTACAAA CTCTTTTTGT TTATTTTTCT AAATACATTC AAATATGTAT
CCGCTCATGA GACAATAACG GCCAAAGAAG AAGATAAAGC TGATGAGCAG TACGATACGG
GTCATGCGAC TGTTAAATCG GTGTCGTTTT CGCATACTGG TTGCCTGACT cacAAAAGGT
TCCTTGAAGT ATGTCCCACG CTGTGGACGG TACTTACATT AAGGCACAAC AGGACAAAAT
GGTCAATTCT TCGTTATGTA AAAAAGCGTC AGTGGATACA TATTTTAATG TTATGGAAGT
TAATTTAATT ATTTACAATG ATGATGTAAT AATGATGAAT TAACATGATA AACGTAATAA
TTCATTAATC AGGGTTATTT TGTTTGATAT ATATCAATTG GTACTTTCAT ATGACACTTA
GAA

What is claimed is:

1. A method of delivering a therapeutic polypeptide to a mammalian subject in need thereof, the method comprising:
a) obtaining a microbiome sample comprising bacterial cells from a donating subject;
b) isolating a bacterial cell from the microbiome sample, wherein the bacteria cell is from a bacterial strain that is commensal/native to the donating subject;
c) culturing the isolated bacteria cells in vitro to yield a substantially homogeneous population of the isolated and cultured bacteria cells;
d) transforming the population of bacterial cells with one or more polynucleotides that are heterologous to the bacteria and/or the donating subject, wherein the one or more polynucleotides encode one or more therapeutic polypeptides; and
e) administering or causing to be administered to a receiving subject at least a portion of the substantially homogeneous and transformed population of the isolated and cultured bacteria cells, wherein the administered bacteria cells are capable of colonizing permanently or long-term in or on the mammalian subject and express the one or more therapeutic polypeptides.

2. The method of claim 1, further comprising the step of determining and/or measuring the colonization or presence of the administered bacteria cells in or on said mammalian subject.

3. The method of any one of claims 1 to 3, wherein the microbiome sample is obtained from a biological sample selected from the group consisting of a bodily excretion (e.g., feces, saliva, mucus, urine, or breath), a biopsy or swab of a surface (e.g., gastrointestinal (GI) tract, oral cavity, pharynx, nasal cavity, urogenital track, skin, anus/rectum, vagina, or eye) and a pathological specimen (e.g., cancerous tissue, amputated limbs, or inflamed organs).

4. The method of any one of claims 1 to 3, wherein the bacterial cell does not comprise a polynucleotide encoding for a pathogenic toxin.

5. The method of claim 4, wherein the bacterial cell does not comprise one or more polynucleotides encoding for one or more pathogenic toxins selected from the group consisting of AB toxin, Alpha toxin, Anthrax toxin, Botulinum toxin, Cereulide, Cholesterol-dependent cytolysin, Clostridial Cytotoxin family, Clostridium botulinum C3 toxin, Clostridium difficile toxin A, Clostridium difficile toxin B, Clostridium enterotoxin, Clostridium perfringens alpha toxin, Clostridium perfringens beta toxin, Cry1Ac, Cry6Aa, Cry34Ab1, Delta endotoxin, Diphtheria toxin, Enterotoxins, Enterotoxin type B, Erythrogenic toxin, Exfoliatin, Fragilysin, Haemolysin E, Heat-labile enterotoxin, Heat-stable enterotoxin, Hemolysin, HrpZ Family, Leukocidin, Listeriolysin O, Panton-Valentine leucocidin, intact Pathogenicity island, Phenol-soluble modulin, Pneumolysin, Pore-forming toxin, Pseudomonas exotoxin, Pyocyanin, anti-eukaryotic Rhs toxins, RTX toxin, Shiga toxins, Shiga-like toxin, Staphylococcus aureus alpha toxin, Staphylococcus aureus beta toxin, Staphylococcus aureus delta toxin, Streptolysin, Tetanolysin, Tetanospasmin, Toxic shock syndrome toxin, Tracheal cytotoxin, and/or Verocytotoxin.

6. The method of any one of claims 1 to 5, wherein the bacterial cell is antibiotic sensitive to one or more antibiotic agents used for selection of transformed bacterial cells, e.g., kanamycin, chloramphenicol, carbenicillin, hygromycin and/or trimethoprim.

7. The method of any one of claims 1 to 6, wherein the bacterial cell is not antibiotic resistant to one or more clinically used antibiotic agents.

8. The method of claim 7, wherein the bacterial cell is not antibiotic resistant to one or more antibiotic agents selected from antibiotic macrolides (e.g., azithromycin, clarithromycin, erythromycin, fidaxomicin, telithromycin, carbomycin A, josamycin, kitasamycin, midecamycin/midecamycin acetate, oleandomycin, solithromycin, spiramycin, troleandomycin, tylosin/tylocine, or roxithromycin), rifamycins (e.g., rifampicin (or rifampin), rifabutin, rifapentine, rifalazil, or rifaximin), polymyxins (e.g., polymyxin B, or polymyxin E (colistin)), quinolone antibiotics (e.g., nalidixic acid, ofloxacin, levofloxacin, ciprofloxacin, norfloxacin, enoxacin, lomefloxacin, grepafloxacin, trovafloxacin, sparfloxacin, temafloxacin, moxifloxacin, gatifloxacin, gemifloxacin), beta-lactams (e.g., penicillin, cloxacillin, dicloxacillin, flucloxacillin, methicillin, nafcillin, oxacillin, temocillin, amoxicillin, ampicillin, mecillinam, carbenicillin, ticarcillin, azlocillin, mezlocillin, or piperacillin), aminoglycosides (e.g., amikacin, gentamicin, neomycin, streptomycin, or tobramycin), cephalosporins (e.g., cefadroxil, cefazolin, cephalexin, cefaclor, cefoxitin, cefprozil, cefuroxime, loracarbef, cefixime, cefdinir, cefditoren, cefoperazone, cefotaxime, cefpodoxime, cefepime, or ceftobiprole), monobactams (e.g., aztreonam, tigemonam, nocardicin A, or tabtoxinine β-lactam), carbapenems (e.g., biapenem, doripenem, ertapenem, faropenem, imipenem, meropenem, panipenem, razupenem, tebipenem, or thienamycin), or tetracyclines (e.g., tetracycline, chlortetracycline, oxytetracycline, demeclocycline, lymecycline, meclocycline, methacycline, minocycline, rolitetracycline, or tigecycline).

9. The method of any one of claims 1 to 8, wherein the one or more heterologous polynucleotides encode a fluorescent protein, e.g., green fluorescent protein, yellow fluorescent protein, red fluorescent protein (mCherry, mEos2, mRuby2, mRuby3, mClover3, mApple, mKate2, mMaple, mCardinal, mNeptune), mTurquoise, or mVenus.

10. The method of any one of claims 1 to 8, wherein the one or more heterologous polynucleotides encode an enzyme, a cytokine or a peptide hormone.

11. The method of claim 10, wherein the enzyme is a bile salt hydrolase, e.g., from Lactobacillus, e.g., bshA (Gene ID 3251811) or bshB (Gene ID 3252955), N-acylphosphatidylethanolamine (NAPE)-hydrolyzing phospholipase D, Actinobacillus actinomycetemcomitans dispersin B (DspB), lactase (beta-galactosidase), an aldehyde dehydrogenase, an alcohol dehydrogenase (e.g., ADH1A, ADH1B, ADH1C, ADH2, ADH3, ADH4, ADH5, ADH6, or ADH7), bile acid-CoA:amino acid N-acyltransferase (BAAT), phenylalanine hydroxylase, butyrate synthesis pathway enzymes, Aspergillus niger-derived prolyl endoprotease (AN-PEP), 7-alpha-hydroxysteroid dehydrogenase (7-alpha-HSDH), 7 beta-hydroxysteroid dehydrogenase (7-beta-HSDH), or cholylglycine hydrolase and cholic acid 7alpha-dehydroxylase.

12. The method of claim 10, wherein the cytokine is selected from mammalian (e.g., human) IL-10, mammalian (e.g., human) IL-27 dimer, (IL27 alpha subunit and/or Epstein-Barr virus induced 3 (EBI3) subunit expressed separately or as a fusion protein), or TGF-β.

13. The method of claim 10, wherein the peptide hormone is selected from the group consisting of mammalian glucagon, glucagon-like peptide 1 (GLP-1), mammalian glucagon-like peptide 2 (GLP-2), Fibroblast growth factor 1 (FGF1), Fibroblast growth factor 15 (FGF15), Fibroblast growth factor 19 (FGF19), insulin, and proinsulin.

14. The method of any one of claims 1 to 8, wherein the one or more heterologous polynucleotides encode Akkermansia muciniphila Amuc_1100, Vibrio vulnificus flagellin B, elafin, trefoil factor 1 (TFF1), trefoil factor 2 (TFF2), trefoil factor 3 (TFF3), anti-TNFα antibodies/nanobodies or fragments or single chains thereof, Nostoc elipsosporum cyanovirin-N or microcin J25 (MccJ25).

15. The method of any one of claims 1 to 14, the one or more heterologous polynucleotides comprise codon bias configured to improve or enhance expression of the heterologous protein in the transformed population of the isolated and cultured bacterial cells.

16. The method of any one of claims 1 to 15, wherein the one or more heterologous polynucleotides are integrated into the chromosome of the bacterial cells of the transformed population.

17. The method of claim 16, wherein the one or more heterologous polynucleotides are integrated into the attB and/or yfgG genes of the bacterial genome.

18. The method of any one of claims 1 to 15, wherein the one or more heterologous polynucleotides are in a plasmid episomally introduced into the bacteria cells bacterial cells of the transformed population.

19. The method of claim 18, wherein the transformed bacterial cells further comprise a plasmid retention or maintenance system, e.g., a partitioning system or a toxin-antitoxin module or system.

20. The method of any one of claims 1 to 19, wherein the one or more heterologous polynucleotides are integrated into an expression cassette having at least or at least about 80%, 85%, 90%, 95%, 97%, 99% or 100% sequence identity to SEQ ID NO:2, and are expressed under the control of a Ptrc promoter.

21. The method of any one of claims 1 to 20, wherein the heterologous polynucleotide is expressed under the control of a constitutive promoter.

22. The method of any one of claims 1 to 20 wherein the heterologous polynucleotide is expressed under the control of an inducible promoter.

23. The method of any one of claims 1 to 22, wherein the bacteria cell is from a gram negative bacterial strain.

24. The method of any one of claims 1 to 23, wherein the bacteria cell is derived from a bacteria genus selected from the group consisting of Bacteroides (e.g., Alistipes, Prevotella, Paraprevotella, Parabacteroides, or Odoribacter), Clostridium, Streptococcus, Lactococcus, Eubacterium rectale, Escherichia coli, Enterobacter sp., Klebsiella sp., Bifidobacterium, Staphylococcus, Lactobacillus, Veillonella, Haemophilus, Moraxella, Corynebacterium and Propionibacterium.

25. The method of any one of claims 1 to 24, wherein the bacteria cell is derived from Escherichia coli.

26. The method of any one of claims 1 to 25, wherein a detectable portion of the administered bacteria cells stably colonize the tissue or surface to which they are administered for at least or at least about 2, 3, 4, 5, 6, 7 days, e.g., at least or at least about 1 week, e.g., at least or at least about 2, 3, 4, 5, 10, 15, 20, 25, 30, 35, 40, 45, 50, 55, 60, 75, 100, 125 weeks, or longer, e.g., for the duration of the life of the subject or for a period that is within a range defined by any two of the aforementioned time periods.

27. The method of any one of claims 1 to 26, wherein a detectable portion of the administered bacteria cells stably and permanently colonize the tissue or surface to which they are administered.

28. The method of any one of claims 1 to 27, wherein at least or at least about 10%, 15%, 20%, 25%, 30%, 35%, 40%, 45%, 50%, 55%, 60%, 65%, 70%, 75%, 80%, 85%, 90%, 95% of the administered bacteria cells stably colonize the tissue or surface to which they are administered.

29. The method of any one of claims 1 to 28, wherein the bacterial cells:
i) are capable of metabolizing one or more carbohydrates selected from the group consisting of sucrose, xylose, d-maltose, N-acetyl-d-glucosamine, d-galactose, and d-ribose;
ii) utilize both glycolytic and gluconeogenic substrates;
iii) are non-motile (e.g, have non-functioning flagella, e.g., due to a mutation in the flhDC operon);
iv) are capable of producing ribose-5-phosphate;
v) are able to grow in defined medium lacking vitamin B12 (cyanocobalamin) (e.g., are demonstrated vitamin B12 prototrophs);
vi) express an UDP-glucose-4-epimerase and/or a glycosyltransferase;
vii) comprise multiple copies of a gene encoding β subunit of tryptophan synthase gene;
viii) comprise multiple copies of a gene encoding propionate CoA-transferase
ix) express capsular polysaccharide (CPS) 4 (CPS4);
x) express an rnf-like oxidoreductase complex;
xi) catabolize tryptophan to yield indole and other indole metabolites, e.g., indole-3-propionate and indole-3-aldehyde; and/or
xii) do not produce any agent that induces double-stranded DNA breaks, e.g., do not have a genomic island that encodes a giant modular nonribosomal peptide and polyketide synthases, express hybrid peptide-polyketide genotoxins, and/or do not have an active clbA gene.

30. The method of any one of claims 1 to 29, wherein the subject is a human.

31. The method of any one of claims 1 to 30, wherein at least or at least about 10⁶, 10⁷, 10⁸, 10⁹, 10¹⁰, 10¹¹, 10¹², 10¹³ bacterial cells are administered.

32. The method of any one of claims 1 to 31, wherein the donating subject and the receiving subject are the same individual, e.g., wherein the microbiome sample is autologous to the subject.

33. The method of any one of claims 1 to 31, wherein the donating subject and the receiving subject are different individuals.

34. The method of any one of claims 1 to 32, wherein the administered bacteria cells are administered to the same tissue or surface from which the microbiome sample was obtained.

35. The method of any one of claims 1 to 34, wherein the microbiome sample is obtained from the skin or the eye, and the administered bacteria cells are topically administered to the subject, e.g., in a buffered suspension, a gel, a lotion, a cream, or an ointment.

36. The method of any one of claims 1 to 34, wherein the microbiome sample is obtained from the nasal cavity, and the administered bacteria cells are administered via nasal gavage.

37. The method of any one of claims 1 to 34, wherein the microbiome sample is obtained from the vagina, and the administered bacteria cells are administered intravaginally.

38. The method of any one of claims 1 to 34, wherein the microbiome sample is obtained from the GI tract, and the administered bacteria cells are administered to the subject orally or rectally.

39. The method of claim 38, wherein the administered bacteria cells are administered orally to the subject via a gastric tube or in an edible composition.

40. The method of claim 39, wherein the edible composition comprises a gel capsule comprising the administered bacteria cells or the administered bacteria cells are encapsulated.

41. The method of claim 39, wherein the edible composition is selected from the group consisting of yogurt, milk, ice cream, vegetable puree, fruit puree, sorbet, and oatmeal.

42. The method of claim 39, wherein the edible composition is a beverage.

43. The method of claim 42, wherein the beverage is a buffered solution.

44. The method of any one of claims 1 to 43, wherein the administered bacteria cells are administered to the subject multiple times, e.g., in daily, weekly, bi-weekly or monthly intervals.

45. The method of any one of claims 1 to 44, wherein the administered bacteria cells are administered to the subject in daily, weekly, bi-weekly or monthly intervals.

46. The method of any one of claims 1 to 45, wherein the administered transformed bacterial cells do not alter the microbiome of the receiving subject.

47. A substantially homogeneous population of bacteria cells commensal to a mammal, wherein the substantially homogeneous population of bacteria is transformed to express one or more polynucleotides that are heterologous to the mammal and/or the bacteria.

48. The substantially homogeneous population of bacteria cells of claim 47, wherein the population of bacteria commensal to the mammal is capable of colonizing or is configured to colonize in or on the mammal permanently or long-term.

49. The substantially homogeneous population of bacteria cells of any one of claims 47 to 48, wherein the population of bacterial cells is capable of colonizing or is configured to colonize in or on the mammal for at least or at least about 2, 3, 4, 5, 6, 7 days, e.g., at least or at least about 1 week, e.g., at least or at least about 2, 3, 4, 5, 10, 15, 20, 25, 30, 35, 40, 45, 50, 55, 60, 75, 100, 125 weeks, or longer, e.g., for the duration of the life of the mammal.

50. The substantially homogeneous population of bacteria cells of any one of claims 47 to 49, wherein the bacterial cells:
i) are capable of metabolizing one or more carbohydrates selected from the group consisting of sucrose, xylose, d-maltose, N-acetyl-d-glucosamine, d-galactose, and d-ribose;
ii) utilize both glycolytic and gluconeogenic substrates;
iii) are non-motile (e.g, have non-functioning flagella, e.g., due to a mutation in the flhDC operon);
iv) are capable of producing ribose-5-phosphate;
v) are able to grow in defined medium lacking vitamin B12 (cyanocobalamin) (e.g., are demonstrated vitamin B12 prototrophs);
vi) express an UDP-glucose-4-epimerase and/or a glycosyltransferase;
vii) comprise multiple copies of a gene encoding β subunit of tryptophan synthase gene;
viii) comprise multiple copies of a gene encoding propionate CoA-transferase
ix) express capsular polysaccharide (CPS) 4 (CPS4);
x) express an rnf-like oxidoreductase complex;
xi) catabolize tryptophan to yield indole and other indole metabolites, e.g., indole-3-propionate and indole-3-aldehyde; and/or
xii) do not produce any agent that induces double-stranded DNA breaks, e.g., do not have a genomic island that encodes a giant modular nonribosomal peptide and polyketide synthases, express hybrid peptide-polyketide genotoxins, and/or do not have an active clbA gene.

51. The substantially homogeneous population of bacteria cells of any one of claims 47 to 50, wherein the population of bacterial cells do not comprise one or more polynucleotides encoding for one or more pathogenic toxins selected from the group consisting of AB toxin, Alpha toxin, Anthrax toxin, Botulinum toxin, Cereulide, Cholesterol-dependent cytolysin, Clostridial Cytotoxin family, Clostridium botulinum C3 toxin, Clostridium difficile toxin A, Clostridium difficile toxin B, Clostridium enterotoxin, Clostridium perfringens alpha toxin, Clostridium perfringens beta toxin, Cry1Ac, Cry6Aa, Cry34Ab1, Delta endotoxin, Diphtheria toxin, Enterotoxins, Enterotoxin type B, Erythrogenic toxin, Exfoliatin, Fragilysin, Haemolysin E, Heat-labile enterotoxin, Heat-stable enterotoxin, Hemolysin, HrpZ Family, Leukocidin, Listeriolysin O, Panton-Valentine leucocidin, intact Pathogenicity island, Phenol-soluble modulin, Pneumolysin, Pore-forming toxin, Pseudomonas exotoxin, Pyocyanin, anti-eukaryotic Rhs toxins, RTX toxin, Shiga toxins, Shiga-like toxin, Staphylococcus aureus alpha toxin, Staphylococcus aureus beta toxin, Staphylococcus aureus delta toxin, Streptolysin, Tetanolysin, Tetanospasmin, Toxic shock syndrome toxin, Tracheal cytotoxin, and/or Verocytotoxin.

52. The substantially homogeneous population of bacteria cells of any one of claims 47 to 51, wherein the population of bacterial cells is antibiotic resistant to one or more antibiotic agents used for selection of the transformed bacterial cells, e.g., kanamycin, chloramphenicol, carbenicillin, hygromycin and/or trimethoprim.

53. The substantially homogeneous population of bacteria cells of any one of claims 47 to 52, wherein the population of bacterial cells is not antibiotic resistant to clinically used antibiotic agents.

54. The substantially homogeneous population of bacteria cells of claim 53, wherein the bacterial cell is not antibiotic resistant to one or more clinically used antibiotic agents selected from antibiotic macrolides (e.g., azithromycin, clarithromycin, erythromycin, fidaxomicin, telithromycin, carbomycin A, josamycin, kitasamycin, midecamycin/midecamycin acetate, oleandomycin, solithromycin, spiramycin, troleandomycin, tylosin/tylocine, or roxithromycin), rifamycins (e.g., rifampicin (or rifampin), rifabutin, rifapentine, rifalazil, or rifaximin), polymyxins (e.g., polymyxin B, or polymyxin E (colistin)), quinolone antibiotics (e.g., nalidixic acid, ofloxacin, levofloxacin, ciprofloxacin, norfloxacin, enoxacin, lomefloxacin, grepafloxacin, trovafloxacin, sparfloxacin, temafloxacin, moxifloxacin, gatifloxacin, or gemifloxacin), beta-lactams (e.g., penicillin, cloxacillin, dicloxacillin, flucloxacillin, methicillin, nafcillin, oxacillin, temocillin, amoxicillin, ampicillin, mecillinam, carbenicillin, ticarcillin, azlocillin, mezlocillin, or piperacillin), aminoglycosides (e.g., amikacin, gentamicin, neomycin, streptomycin, or tobramycin), cephalosporins (e.g., cefadroxil, cefazolin, cephalexin, cefaclor, cefoxitin, cefprozil, cefuroxime, loracarbef, cefixime, cefdinir, cefditoren, cefoperazone, cefotaxime, cefpodoxime, cefepime, or ceftobiprole), monobactams (e.g., aztreonam, tigemonam, nocardicin A, or tabtoxinine β-lactam), carbapenems (e.g., biapenem, doripenem, ertapenem, faropenem, imipenem, meropenem, panipenem, razupenem, tebipenem, or thienamycin), or tetracyclines (e.g., tetracycline, chlortetracycline, oxytetracycline, demeclocycline, lymecycline, meclocycline, methacycline, minocycline, rolitetracycline, or tigecycline).

55. The substantially homogeneous population of bacteria cells of any one of claims 47 to 54, wherein the one or more heterologous polynucleotides encode a fluorescent protein, e.g., green fluorescent protein, yellow fluorescent protein, red fluorescent protein (mCherry, mEos2, mRuby2, mRuby3, mClover3, mApple, mKate2, mMaple, mCardinal, or mNeptune), mTurquoise, or mVenus.

56. The substantially homogeneous population of bacteria cells of any one of claims 47 to 55, wherein the one or more heterologous polynucleotides encode an enzyme, a cytokine or a peptide hormone.

57. The substantially homogeneous population of bacteria cells of claim 56, wherein the enzyme is a bile salt hydrolase, e.g., from Lactobacillus, e.g., bshA (Gene ID 3251811) or bshB (Gene ID 3252955), N-acylphosphatidylethanolamine (NAPE)-hydrolyzing phospholipase D, Actinobacillus actinomycetemcomitans dispersin B (DspB), lactase (beta-galactosidase), an aldehyde dehydrogenase, an alcohol dehydrogenase (e.g., ADH1A, ADH1B, ADH1C, ADH2, ADH3, ADH4, ADH5, ADH6, or ADH7), bile acid-CoA:amino acid N-acyltransferase (BAAT), phenylalanine hydroxylase, butyrate synthesis pathway enzymes, Aspergillus niger-derived prolyl endoprotease (AN-PEP), 7-alpha-hydroxysteroid dehydrogenase (7-alpha-HSDH), 7 beta-hydroxysteroid dehydrogenase (7-beta-HSDH), or cholylglycine hydrolase and cholic acid 7alpha-dehydroxylase.

58. The substantially homogeneous population of bacteria cells of claim 56, wherein the cytokine is selected from the group consisting of mammalian (e.g., human) IL-10, mammalian (e.g., human) IL-27 dimer (IL27 alpha subunit and/or Epstein-Barr virus induced 3 (EBI3) subunit expressed separately or as a fusion protein), and TGF-β.

59. The substantially homogeneous population of bacteria cells of claim 56, wherein the peptide hormone is selected from the group consisting of mammalian glucagon, glucagon-like peptide 1 (GLP-1), mammalian glucagon-like peptide 2 (GLP-2), Fibroblast growth factor 1 (FGF1), Fibroblast growth factor 15 (FGF15), Fibroblast growth factor 19 (FGF19), insulin, and proinsulin.

60. The substantially homogeneous population of bacteria cells of any one of claims 47 to 59, wherein the one or more heterologous polynucleotides encode Akkermansia muciniphila Amuc_1100, Vibrio vulnificus flagellin B, elafin, trefoil factor 1 (TFF1), trefoil factor 2 (TFF2), trefoil factor 3 (TFF3), anti-TNFα antibodies/nanobodies or fragments or single chains thereof, Nostoc elipsosporum cyanovirin-N or microcin J25 (MccJ25).

61. The substantially homogeneous population of bacteria cells of any one of claims 47 to 60, wherein the one or more heterologous polynucleotides comprise codon bias configured to improve or enhance expression of the heterologous protein in the transformed population of the isolated and cultured bacterial cells.

62. The substantially homogeneous population of bacteria cells of any one of claims 47 to 61, wherein the one or more heterologous polynucleotides are integrated into the chromosome of the bacterial cells of the transformed population.

63. The substantially homogeneous population of bacteria cells of claim 62, wherein the one or more heterologous polynucleotides are integrated into the attB and/or yfgG genes of the bacterial genome.

64. The substantially homogeneous population of bacteria cells of any one of claims 47 to 61, wherein the heterologous polynucleotide is in a plasmid episomally located in the bacterial cells.

65. The substantially homogeneous population of bacteria cells of claim 64, wherein the population of bacterial cells further comprise a plasmid retention or maintenance system, e.g., a partitioning system or a toxin-antitoxin module or system.

66. The substantially homogeneous population of bacteria cells of any one of claims 47 to 64, wherein the one or more heterologous polynucleotides are integrated into an expression cassette having at least or at least about 80%, 85%, 90%, 95%, 97%, 99% or 100% sequence identity to SEQ ID NO:2, and are expressed under the control of a Ptrc promoter.

67. The substantially homogeneous population of bacteria cells of any one of claims 47 to 66, wherein the substantially homogenous population of bacterial cells is from a gram negative bacterial strain.

68. The substantially homogeneous population of bacteria cells of any one of claims 47 to 67, wherein the substantially homogenous population of bacterial cells is derived from a bacteria genus selected from the group consisting of Bacteroides (e.g., Alistipes, Prevotella, Paraprevotella, Parabacteroides, or Odoribacter), Clostridium, Streptococcus, Lactococcus, Eubacterium rectale, Escherichia coli, Enterobacter sp., Klebsiella sp., Bifidobacterium, Staphylococcus, Lactobacillus, Veillonella, Haemophilus, Moraxella, Corynebacterium and Propionibacterium.

69. The substantially homogeneous population of bacteria cells of any one of claims 47 to 68, wherein the substantially homogenous population of bacterial cells is derived from Escherichia coli.

70. The substantially homogeneous population of bacteria cells of any one of claims 47 to 69, wherein the population of bacteria cells is lyophilized or cryopreserved.

71. A pharmaceutical composition suitable for administration to a mammal comprising a substantially homogeneous population of bacteria cells commensal to the mammal, wherein the population of bacteria is transformed to express one or more polynucleotides that are heterologous to the mammal and/or the bacteria.

72. A composition edible by a mammal comprising a substantially homogeneous population of bacteria cells commensal to the mammal, wherein the substantially homogenous population of bacteria cells is transformed to express one or more polynucleotides that are heterologous to the mammal and/or the bacteria.

73. The composition of any one of claims 71 to 72, wherein the substantially homogenous population of bacteria cells commensal to the mammal is produced according to a method of any one of claims 1 to 44.

74. The composition of any one of claims 71 to 73, wherein the substantially homogenous population of bacteria cells commensal to the mammal is capable of colonizing or is configured to colonize in or on the mammal permanently or long-term.

75. The composition of any one of claims 71 to 74, wherein the population of bacterial cells is capable of colonizing or is configured to colonize in or on the mammal for at least or at least about 2, 3, 4, 5, 6, 7 days, e.g., at least or at least about 1 week, e.g., at least or at least about 2, 3, 4, 5, 10, 15, 20, 25, 30, 35, 40, 45, 50, 55, 60, 75, 100, 125 weeks, or longer, e.g., for the duration of the life of the mammal.

76. The composition of any one of claims 71 to 75, wherein the bacterial cells:
i) are capable of metabolizing one or more carbohydrates selected from the group consisting of sucrose, xylose, d-maltose, N-acetyl-d-glucosamine, d-galactose, and d-ribose;
ii) utilize both glycolytic and gluconeogenic substrates;
iii) are non-motile (e.g, have non-functioning flagella, e.g., due to a mutation in the flhDC operon);
iv) are capable of producing ribose-5-phosphate;
v) are able to grow in defined medium lacking vitamin B12 (cyanocobalamin) (e.g., are demonstrated vitamin B12 prototrophs);
vi) express an UDP-glucose-4-epimerase and/or a glycosyltransferase;
vii) comprise multiple copies of a gene encoding β subunit of tryptophan synthase gene;
viii) comprise multiple copies of a gene encoding propionate CoA-transferase
ix) express capsular polysaccharide (CPS) 4 (CPS4);
x) express an rnf-like oxidoreductase complex;
xi) catabolize tryptophan to yield indole and other indole metabolites, e.g., indole-3-propionate and indole-3-aldehyde; and/or
xii) do not produce any agent that induces double-stranded DNA breaks, e.g., do not have a genomic island that encodes a giant modular nonribosomal peptide and polyketide synthases, express hybrid peptide-polyketide genotoxins, and/or do not have an active clbA gene.

77. The composition of any one of claims 71 to 76, wherein the substantially homogenous population of bacteria cells do not comprise one or more polynucleotides encoding for one or more pathogenic toxins selected from the group consisting of AB toxin, Alpha toxin, Anthrax toxin, Botulinum toxin, Cereulide, Cholesterol-dependent cytolysin, Clostridial Cytotoxin family, Clostridium botulinum C3 toxin, Clostridium difficile toxin A, Clostridium difficile toxin B, Clostridium enterotoxin, Clostridium perfringens alpha toxin, Clostridium perfringens beta toxin, Cry1Ac, Cry6Aa, Cry34Ab1, Delta endotoxin, Diphtheria toxin, Enterotoxins, Enterotoxin type B, Erythrogenic toxin, Exfoliatin, Fragilysin, Haemolysin E, Heat-labile enterotoxin, Heat-stable enterotoxin, Hemolysin, HrpZ Family, Leukocidin, Listeriolysin O, Panton-Valentine leucocidin, intact Pathogenicity island, Phenol-soluble modulin, Pneumolysin, Pore-forming toxin, Pseudomonas exotoxin, Pyocyanin, anti-eukaryotic Rhs toxins, RTX toxin, Shiga toxins, Shiga-like toxin, Staphylococcus aureus alpha toxin, Staphylococcus aureus beta toxin, Staphylococcus aureus delta toxin, Streptolysin, Tetanolysin, Tetanospasmin, Toxic shock syndrome toxin, Tracheal cytotoxin, and/or Verocytotoxin.

78. The composition of any one of claims 71 to 77, wherein the substantially homogenous population of bacteria cells is antibiotic resistant to one or more antibiotic agents used for selection of the transformed bacterial cells, e.g., kanamycin, chloramphenicol, carbenicillin, hygromycin and/or trimethoprim.

79. The composition of any one of claims 71 to 78, wherein the bacterial cell is not antibiotic resistant to clinically used antibiotic agents.

80. The composition of claim 79, wherein the bacterial cell is not antibiotic resistant to one or more clinically used antibiotic agents selected from antibiotic macrolides (e.g., azithromycin, clarithromycin, erythromycin, fidaxomicin, telithromycin, carbomycin A, josamycin, kitasamycin, midecamycin/midecamycin acetate, oleandomycin, solithromycin, spiramycin, troleandomycin, tylosin/tylocine, or roxithromycin), rifamycins (e.g., rifampicin (or rifampin), rifabutin, rifapentine, rifalazil, or rifaximin), polymyxins (e.g., polymyxin B, or polymyxin E (colistin)), quinolone antibiotics (e.g., nalidixic acid, ofloxacin, levofloxacin, ciprofloxacin, norfloxacin, enoxacin, lomefloxacin, grepafloxacin, trovafloxacin, sparfloxacin, temafloxacin, moxifloxacin, gatifloxacin, or gemifloxacin), beta-lactams (e.g., penicillin, cloxacillin, dicloxacillin, flucloxacillin, methicillin, nafcillin, oxacillin, temocillin, amoxicillin, ampicillin, mecillinam, carbenicillin, ticarcillin, azlocillin, mezlocillin, or piperacillin), aminoglycosides (e.g., amikacin, gentamicin, neomycin, streptomycin, or tobramycin), cephalosporins (e.g., cefadroxil, cefazolin, cephalexin, cefaclor, cefoxitin, cefprozil, cefuroxime, loracarbef, cefixime, cefdinir, cefditoren, cefoperazone, cefotaxime, cefpodoxime, cefepime, or ceftobiprole), monobactams (e.g., aztreonam, tigemonam, nocardicin A, or tabtoxinine β-lactam), carbapenems (e.g., biapenem, doripenem, ertapenem, faropenem, imipenem, meropenem, panipenem, razupenem, tebipenem, or thienamycin), or tetracyclines (e.g., tetracycline, chlortetracycline, oxytetracycline, demeclocycline, lymecycline, meclocycline, methacycline, minocycline, rolitetracycline, or tigecycline).

81. The composition of any one of claims 71 to 80, wherein the one or more heterologous polynucleotides encode a fluorescent protein, e.g., green fluorescent protein, yellow fluorescent protein, red fluorescent protein (mCherry, mEos2, mRuby2, mRuby3, mClover3, mApple, mKate2, mMaple, mCardinal, or mNeptune), mTurquoise, or mVenus.

82. The composition of any one of claims 71 to 81, wherein the one or more heterologous polynucleotides encode an enzyme, a cytokine or a peptide hormone.

83. The composition of claim 82, wherein the enzyme is a bile salt hydrolase, e.g., from Lactobacillus, e.g., bshA (Gene ID 3251811) or bshB (Gene ID 3252955), N-acylphosphatidylethanolamine (NAPE)-hydrolyzing phospholipase D, Actinobacillus actinomycetemcomitans dispersin B (DspB), lactase (beta-galactosidase), an aldehyde dehydrogenase, an alcohol dehydrogenase (e.g., ADH1A, ADH1B, ADH1C, ADH2, ADH3, ADH4, ADH5, ADH6, or ADH7), bile acid-CoA:amino acid N-acyltransferase (BAAT), phenylalanine hydroxylase, butyrate synthesis pathway enzymes, Aspergillus niger-derived prolyl endoprotease (AN-PEP), 7-alpha-hydroxysteroid dehydrogenase (7-alpha-HSDH), 7 beta-hydroxysteroid dehydrogenase (7-beta-HSDH), or cholylglycine hydrolase and cholic acid 7alpha-dehydroxylase.

84. The composition of claim 82, wherein the cytokine is selected from the group consisting of mammalian (e.g., human) IL-10, mammalian (e.g., human) IL-27 dimer (IL27 alpha subunit and/or Epstein-Barr virus induced 3 (EBI3) subunit expressed separately or as a fusion protein), and TGF-β.

85. The composition of claim 82, wherein the peptide hormone is selected from the group consisting of mammalian glucagon, glucagon-like peptide 1 (GLP-1), mammalian glucagon-like peptide 2 (GLP-2), Fibroblast growth factor 1 (FGF1), Fibroblast growth factor 15 (FGF15), Fibroblast growth factor 19 (FGF19), insulin, and proinsulin.

86. The composition of any one of claims 71 to 81, wherein the one or more heterologous polynucleotides encode Akkermansia muciniphila Amuc_1100, Vibrio vulnificus flagellin B, elafin, trefoil factor 1 (TFF1), trefoil factor 2 (TFF2), trefoil factor 3 (TFF3), anti-TNFα antibodies/nanobodies or fragments or single chains thereof, Nostoc elipsosporum cyanovirin-N or microcin J25 (MccJ25).

87. The composition of any one of claims 71 to 86, the one or more heterologous polynucleotides comprise codon bias configured to improve or enhance expression of the heterologous protein in the transformed population of the isolated and cultured bacterial cells.

88. The composition of any one of claims 71 to 87, wherein the one or more heterologous polynucleotides are integrated into the chromosome of the bacterial cells of the transformed population.

89. The composition of claim 88, wherein the one or more heterologous polynucleotides are integrated into the attB and/or yfgG genes of the bacterial genome.

90. The composition of any one of claims 71 to 87, wherein the heterologous polynucleotide is in a plasmid episomally located in the bacterial cells.

91. The composition of any one of claims 71 to 90, wherein the one or more heterologous polynucleotides are integrated into an expression cassette having at least or at least about 80%, 85%, 90%, 95%, 97%, 99% or 100% sequence identity to SEQ ID NO:2, and are expressed under the control of a Ptrc promoter.

92. The composition of any one of claims 71 to 91, wherein the substantially homogenous population of bacterial cells is from a gram negative bacterial strain.

93. The composition of any one of claims 71 to 92, wherein the substantially homogenous population of bacterial cells is derived from a bacteria genus selected from the group consisting of Bacteroides (e.g., Alistipes, Prevotella, Paraprevotella, Parabacteroides, or Odoribacter), Clostridium, Streptococcus, Lactococcus, Eubacterium rectale, Escherichia coli, Enterobacter sp., Klebsiella sp., Bifidobacterium, Staphylococcus, Lactobacillus, Veillonella, Haemophilus, Moraxella, Corynebacterium and Propionibacterium.

94. The composition of any one of claims 71 to 93, wherein the substantially homogenous population of bacterial cells is derived from Escherichia coli.

95. The composition of any one of claims 71 to 94, wherein the composition comprises a buffered solution or buffered suspension.

96. The composition of claim 72, wherein the edible composition comprises a gel capsule comprising the substantially homogenous population bacteria cells or the administered bacteria cells are encapsulated.

97. The composition of claim 72, wherein the edible composition comprises a beverage.

98. The composition of claim 72, wherein the edible composition is selected from the group consisting of yogurt, milk, ice cream, vegetable puree, fruit puree, sorbet and oatmeal.

99. A kit comprising one or more containers comprising one or more compositions of any one of claims 71 to 98.

100. The kit of claim 99, wherein the substantially homogenous population of bacteria cells are lyophilized.

101. The substantially homogeneous population of bacteria cells of any one of claims 47 to 70, the pharmaceutical composition of claim 71, the composition of any one of claims 72 to 98, or the kit of claims 99 to 100, for use as a medicament.

102. The substantially homogeneous population of bacteria cells of any one of claims 47 to 70, the pharmaceutical composition of claim 71, the composition of any one of claims 72 to 98, or the kit of claims 99 to 100, for use in treating, ameliorating, preventing or inhibiting obesity, diabetes, cancer, such as oral cancer, esophageal cancer, stomach cancer, colon cancer, or rectal cancer, ulcerative colitis, Crohn's disease, HIV, a pathogenic infection, such as Pseudomonas, Clostridia, or Salmonella infection, malnutrition, lactose intolerance, phenylketonuria, Celiac disease, or brain injury, such as traumatic brain injury, a neuropathy, dementia, stroke, or encephalopathy, hypercholesterolemia, male infertility, female infertility or chronic kidney disease.
